(19) United States
(12) Patent Application Publication
(10) Pub. No.: US 2007/0032469 A1
(43) Pub. Date: Feb. 8, 2007

(54) Tricyclic benzimidazoles and their use as metabotropic glutamate receptor modulators

(75) Inventors: Methvin Isaac, Toronto (CA); Abdelmalik Slassi, Toronto (CA); Ian Egle, Toronto (CA); Fupeng Ma, Toronto (CA)

Correspondence Address: BIRCH, STEWART, KOLASCH & BIRCH, LLP, P.O. BOX 747, 8110 GATEHOUSE ROAD, SUITE 500 EAST, FALLS CHURCH, VA, 22040-0747, US

(73) Assignees: AstraZeneca AB, Sodertalje (SE); NPS PHARMACEUTICALS, Salt Lake City, UT (US)

(21) Appl. No.: 11/490,090

(22) Filed: Jul. 21, 2006

Related U.S. Application Data
(60) Provisional application No. 60/705,471, filed on Aug. 5, 2005.

(51) Int. Cl. A61K 31/55; A61K 31/542; A61K 31/5383; A61K 31/519; C07D 498/14; C07D 491/14; C07D 487/14

(52) U.S. Cl. 514/210.21; 544/32; 544/89; 544/250; 514/224.5; 514/229.8; 514/267; 514/211.15; 514/217.05; 514/217.06; 514/227.8

(57) ABSTRACT

The invention provides for a compound of formula (I) or a pharmaceutically acceptable salt thereof: 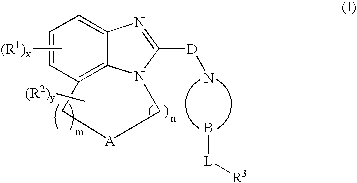
wherein R¹, R², R³, A, B, D, m, n, x, and y are defined as described in the specification. The invention additionally provides a pharmaceutically composition comprising the compound of formula I, together with a method of using the same to treat or prevent neurological and psychiatric disorders. The compounds are useful in therapy related to the treatment or prevention of mGluR2 receptor-mediated disorders.

### BACKGROUND OF THE INVENTION

The present invention relates to novel compounds that function as modulators of glutamate receptors, methods for their preparation, pharmaceutical compositions containing them and their use in therapy.

The metabotropic glutamate receptors (mGluR) constitute a family of GTP-binding-protein (G-protein) coupled receptors that are activated by glutamate, and have important roles in synaptic activity in the central nervous system, including neural plasticity, neural development and neurodegeneration.

Activation of mGluRs in intact mammalian neurons elicits one or more of the following responses: activation of phospholipase C; increases in phosphoinositide (PI) hydrolysis; intracellular calcium release; activation of phospholipase D; activation or inhibition of adenyl cyclase; increases or decreases in the formation of cyclic adenosine monophosphate (cAMP); activation of guanylyl cyclase; increases in the formation of cyclic guanosine monophosphate (cGMP); activation of phospholipase A₂; increases in arachidonic acid release; and increases or decreases in the activity of voltage- and ligand-gated ion channels (Schoepp et al., 1993, Trends Pharmacol. Sci., 14:13 ; Schoepp, 1994, Neurochem. Int., 24:439; Pin et al., 1995, Neuropharmacology 34:1; Bordi & Ugolini, 1999, Prog. Neurobiol. 59:55).

Eight mGluR subtypes have been identified, which are divided into three groups based upon primary sequence similarity, signal transduction linkages, and pharmacological profile. Group-I includes mGluR1 and mGluR5, which activate phospholipase C and the generation of an intracellular calcium signal. The Group-II (mGluR2 and mGluR3) and Group-III (mGluR4, mGluR6, mGluR7, and mGluR8) mGluRs mediate an inhibition of adenylyl cyclase activity and cyclic AMP levels. For a review, see Pin et al., 1999, Eur. J. Pharmacol., 375:277-294.

Members of the mGluR family of receptors are implicated in a number of normal processes in the mammalian CNS, and are important targets for compounds for the treatment of a variety of neurological and psychiatric disorders. Activation of mGluRs is required for induction of hippocampal long-term potentiation and cerebellar long-term depression (Bashir et al., 1993, Nature, 363:347 ; Bortolotto et al., 1994, Nature, 368:740 ; Aiba et al., 1994, Cell, 79:365 ; Aiba et al., 1994, Cell, 79:377). A role for mGluR activation in nociception and analgesia also has been demonstrated (Meller et al., 1993, Neuroreport, 4: 879; Bordi & Ugolini, 1999, Brain Res., 871:223). In addition, mGluR activation has been suggested to play a modulatory role in a variety of other normal processes including synaptic transmission, neuronal development, apoptotic neuronal death, synaptic plasticity, spatial learning, olfactory memory, central control of cardiac activity, waking, motor control and control of the vestibulo-ocular reflex (Nakanishi, 1994, Neuron, 13:1031; Pin et al., 1995, Neuropharmacology, supra; Knopfel et al., 1995, J. Med. Chem., 38:1417).

Recent advances in the elucidation of the neurophysiological roles of mGluRs have established these receptors as promising drug targets in the therapy of acute and chronic neurological and psychiatric disorders and chronic and acute pain disorders. Because of the physiological and pathophysiological significance of the mGluRs, there is a need for new drugs and compounds that can modulate mGluR function.

### SUMMARY OF THE INVENTION

One embodiment of the invention relates to a compound of formula (I) or a pharmaceutically acceptable salt thereof:

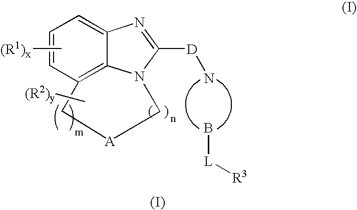

wherein
A is selected from the group consisting of CR⁸R⁹, NR⁵, O, S, SO and SO₂;
B is selected from the group consisting of CH and N;
D is selected from the group consisting of NH, N—C₁₋₆-alkyl, and —(CR⁵R⁶)_{z}—, wherein one of the —CR⁵R⁶— groups may be replaced by —C(O)—, NH, or NC₁₋₆-alkyl;
L is selected from the group consisting of a direct bond and —(CR⁵R⁶)_{w}—, wherein when L is —(CR⁵R⁶)_{w}—:
   (i) B-L may be unsaturated, or two adjacent carbon atoms may form part of a cyclopropyl ring; or
   (ii) one or two CR⁵R⁶ groups may be replaced with O, S, or NR⁵; 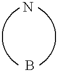
      represents a ring selected from the group consisting of azetidine and a 5- to 7-membered ring, which may be unsaturated, wherein the ring may be substituted by one or more R⁴;
R¹, in each instance, is selected from the group consisting of H, F, Cl, Br, I, OH, CN, nitro, C₁₋₆-alkyl, OC₁₋₆-alkyl, C₁₋₆-alkylhalo, OC₁₋₆-alkylhalo, C₂₋₆-alkenyl, OC₂-₆-alkenyl, C₂₋₆-alkynyl, OC₂₋₆-alkynyl, C₃₋₈-cycloalkyl, C₁₋₆-alkylene-C₃₋₈-cycloalkyl, OC₀₋₆-alkylene-C₃₋₈-cycloalkyl, aryl, heteroaryl, C₁₋₆-alkylenearyl, C₁₋₆-alkyleneheteroaryl, OC₁₋₆-alkylenearyl, OC₁₋₆-alkyleneheteroaryl, C₁₋₆-alkyleneheterocycloalkyl, (CO)R⁵, (CO)OR⁵, C₁₋₆-alkyleneOR⁵, OC₂₋₆-alkyleneOR⁵, C₁₋₆-alkylene(CO)R⁵, OC₁₋₆-alkylene(CO)R⁵, C₁₋₆-alkylenecyano, OC₂₋₆-alkylenecyano, C₀₋₆-alkyleneNR⁶R⁷, OC₂₋₆-alkyleneNR⁶R⁷, C₁₋₆-alkylene(CO)NR⁶R⁷, OC₁₋₆-alkylene(CO)NR⁶R⁷, C₀₋₆-alkyleneNR⁶(CO)R⁷, OC₂₋₆-alkyleneNR⁶(CO)R⁷, C₀₋₆-alkyleneNR⁶(CO)NR⁶R⁷, C₀₋₆-alkyleneSO₂R⁵, OC₂₋₆-alkyleneSO₂R⁵, C₀₋₆-alkylene(SO₂)NR⁶R⁷, OC₂₋₆-alkylene(SO₂)NR⁶R⁷, C₀₋₆-alkyleneNR⁶(SO₂)R⁷, OC₂₋₆-alkyleneNR⁶(SO₂)R⁷, C₀₋₆-alkyleneNR⁶(SO₂)NR⁶R⁷, OC₂₋₆-alkyleneNR⁶(SO₂)NR⁶R⁷, (CO)NR⁶R⁷and SO₃R⁵, wherein any cyclic group may be further substituted with one or more R²;
R² and R⁴, in each instance, are independently selected from the group consisting of H, F, Cl, Br, I, CN, nitro, hydroxy, oxo, C₁₋₆-alkyl, OC₁₋₆-alkyl, C₁₋₆-alkylhalo, OC₁₋₆-alkylhalo, and C₀₋₆-alkyleneNR⁵R⁶;
R³ is a 5- to 12-membered ring system that is optionally substituted by up to three R¹ groups, wherein the ring system may contain one or more heteroatoms independently selected from the group consisting of N, O and S;
R⁵ is selected from the group consisting of H, C₁₋₆-alkyl, aryl, C₃₋₈-cycloalkyl, C₁₋₆-alkylenearyl and C₁₋₆-alkylene-C₃₋₈-cycloalkyl, wherein any cyclic group may be further substituted with one or more independently-selected R² groups;
R⁶ and R⁷ are independently selected from the group consisting of H and C₁₋₆-alkyl;
R⁸ and R⁹ are independently selected from the group consisting of H, —O—(CH₂)₂—O— and —O—(CH₂)₃—O—;
m and n are integers independently selected from the group consisting of 0, 1, 2, 3 and 4, with the proviso that m and n cannot simultaneously be 0;
x and y are integers independently selected from the group consisting of 1, 2, and 3; and
w and z are integers independently selected from the group consisting of 1, 2, 3, 4, 5, and 6;
   or a pharmaceutically-acceptable salt, hydrate, solvate, isoform, tautomer, optical isomer, or combination thereof.

The invention also provides, in addition to a compound of formula I, a pharmaceutically acceptable salt, hydrate, solvate, optical isomer, or combination thereof.

Another embodiment of the invention is to provide a pharmaceutical composition comprising a compound according to formula I together with a pharmaceutically acceptable carrier or excipient.

Yet another embodiment of the invention is a method for treating or preventing a neurological and psychiatric disorder that is associated with glutamate dysfunction. The method comprises the step of administering, to a subject in need of the treatment, a therapeutically effective amount of a compound of formula I, typically in the form of a pharmaceutical composition thereof.

Still another embodiment of the invention is the use of a compound according to formula I, or a pharmaceutically acceptable salt or solvate thereof, for the manufacture of a medicament for the treatment of any of the conditions discussed herein.

Another embodiment of the invention provides a compound of formula I, or a pharmaceutically acceptable salt or solvate thereof, for use in therapy.

The invention additionally provides a process for the preparation of compounds of formula I. General and specific processes are discussed in more detail below.

### DETAILED DESCRIPTION OF THE PREFERRED EMBODIMENTS

The present invention relates to the discovery of compounds that exhibit activity as pharmaceuticals, in particular as modulators of metabotropic glutamate receptors. More particularly, the compounds of the present invention exhibit activity as potentiators, more particularly positive allosteric modulators, of the mGluR2 receptor, and are useful in therapy, in particular for the treatment of neurological and psychiatric disorders associated with glutamate dysfunction.

Definitions

Unless specified otherwise within this specification, the nomenclature used in this specification generally follows the examples and rules stated in NOMENCLATURE OF ORGANIC CHEMISTRY (Pergamon Press, 1979), Sections A, B, C, D, E, F, and H. Optionally, a name of a compound may be generated using a chemical naming program: ACD/ChemSketch, Version 5.09/September 2001, Advanced Chemistry Development, Inc., Toronto, Canada.

The term “C₁₋₆-alkyl” as used herein means a straight- or branched-chain hydrocarbon radical having from one to six carbon atoms, and includes methyl, ethyl, propyl, isopropyl, t-butyl and the like.

The term “C₂₋₆alkenyl” as used herein means a straight- or branched-chain alkenyl radical having from two to six carbon atoms, and includes ethenyl, 1-propenyl, 1-butenyl and the like.

The term “C₂₋₆alkynyl” as used herein means a straight- or branched-chain alkynyl radical having from two to six carbon atoms, and includes 1-propynyl(propargyl), 1-butynyl and the like.

The term “C₃₋₈cycloalkyl” as used herein means a cyclic group (which may be unsaturated) having from three to eight carbon atoms, and includes cyclopropyl, cyclohexyl, cyclohexenyl and the like.

The term “heterocycloalkyl” as used herein means a three- to eight-membered cyclic group (which may be unsaturated) having at least one heteroatom selected from the group consisting of N, S and O, and includes piperidinyl, piperazinyl, pyrrolidinyl, tetrahydrofuranyl and the like.

The term “alkoxy” as used herein means a straight- or branched-chain alkoxy radical having from one to six carbon atoms and includes methoxy, ethoxy, propyloxy, isopropyloxy, t-butoxy and the like.

The term “halo” as used herein means halogen and includes fluoro, chloro, bromo, iodo and the like, in both radioactive and non-radioactive forms.

The term “alkylene” as used herein means a difunctional branched or unbranched saturated hydrocarbon radical having one to six carbon atoms, and includes methylene, ethylene, n-propylene, n-butylene and the like.

The term “alkenylene” as used herein means a difunctional branched or unbranched hydrocarbon radical having two to six carbon atoms and having at least one double bond, and includes ethenylene, n-propenylene, n-butenylene and the like.

The term “alkynylene” as used herein means a difunctional branched or unbranched hydrocarbon radical having two to six carbon atoms and having at least one triple bond, and includes ethynylene, n-propynylene, n-butynylene and the like.

The term “aryl” as used herein means an aromatic group having five to twelve atoms, and includes phenyl, naphthyl and the like.

The term “heteroaryl” means an aromatic group which includes at least one heteroatom selected from the group consisting of N, S and O, and includes groups and includes pyridyl, indolyl, furyl, benzofuryl, thienyl, benzothienyl, quinolyl, oxazolyl and the like.

The terms “alkylaryl”, “alkylheteroaryl” and “alkylcycloalkyl” refer to an alkyl radical substituted with an aryl, heteroaryl or cycloalkyl group, and includes 2-phenethyl, 3-cyclohexyl propyl and the like.

The term “5- to 12-membered ring system . . . wherein the ring system may contain one or more heteroatoms independently selected from N, O or S” includes aromatic and heteroaromatic rings, as well as carbocyclic and heterocyclic rings which may be saturated or unsaturated, and which may be mono-, bi- or tri-cyclic, and includes furyl, isoxazolyl, oxazolyl, pyridyl, pyrimidyl, pyrrolyl, thiazolyl, thienyl, triazolyl, morpholinyl, piperazinyl, piperidyl, tetrahydropyranyl, phenyl, cyclohexyl, cyclopentyl, cyclohexanyl, naphthyl, quinolinyl, indolyl, norbornyl, azabicyclooctyl, adamantyl and the like.

The term “pharmaceutically acceptable salt” means either an acid addition salt or a basic addition salt which is compatible with the treatment of patients.

A “pharmaceutically acceptable acid addition salt” is any non-toxic organic or inorganic acid addition salt of the base compounds represented by Formula I or any of its intermediates. Illustrative inorganic acids which form suitable salts include hydrochloric, hydrobromic, sulfuric and phosphoric acid and acid metal salts such as sodium monohydrogen orthophosphate and potassium hydrogen sulfate. Illustrative organic acids which form suitable salts include the mono-, di- and tricarboxylic acids. Illustrative of such acids are, for example, acetic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, salicylic, 2-phenoxybenzoic, p-toluenesulfonic acid and other sulfonic acids such as methanesulfonic acid and 2-hydroxyethanesulfonic acid. Either the mono- or di-acid salts can be formed, and such salts can exist in either a hydrated, solvated or substantially anhydrous form. In general, the acid addition salts of these compounds are more soluble in water and various hydrophilic organic solvents, and generally demonstrate higher melting points in comparison to their free base forms. The selection criteria for the appropriate salt will be known to one skilled in the art. Other non-pharmaceutically acceptable salts e.g. oxalates may be used for example in the isolation of compounds of Formula I for laboratory use, or for subsequent conversion to a pharmaceutically acceptable acid addition salt.

A “pharmaceutically acceptable basic addition salt” is any non-toxic organic or inorganic base addition salt of the acid compounds represented by Formula I or any of its intermediates. Illustrative inorganic bases which form suitable salts include lithium, sodium, potassium, calcium, magnesium or barium hydroxides. Illustrative organic bases which form suitable salts include aliphatic, alicyclic or aromatic organic amines such as methylamine, trimethyl amine and picoline or ammonia. The selection of the appropriate salt may be important so that an ester functionality, if any, elsewhere in the molecule is not hydrolyzed. The selection criteria for the appropriate salt will be known to one skilled in the art.

The term “solvate” means a compound of Formula I or the pharmaceutically acceptable salt of a compound of Formula I wherein molecules of a suitable solvent are incorporated in a crystal lattice. A suitable solvent is physiologically tolerable at the dosage administered as the solvate. Examples of suitable solvents are ethanol, water and the like. When water is the solvent, the molecule is referred to as a hydrate.

The term “stereoisomers” is a general term for all isomers of the individual molecules that differ only in the orientation of their atoms in space. It includes mirror image isomers (enantiomers), geometric (cis/trans) isomers and isomers of compounds with more than one chiral centre that are not mirror images of one another (diastereomers).

The term “treat” or “treating” means to alleviate symptoms, eliminate the causation of the symptoms either on a temporary or permanent basis, or to prevent or slow the appearance of symptoms of the named disorder or condition.

The term “therapeutically effective amount” means an amount of the compound which is effective in treating the named disorder or condition.

The term “pharmaceutically acceptable carrier” means a non-toxic solvent, dispersant, excipient, adjuvant or other material which is mixed with the active ingredient in order to permit the formation of a pharmaceutical composition, i.e., a dosage form capable of administration to the patient. One example of such a carrier is a pharmaceutically acceptable oil typically used for parenteral administration.

Compounds of the invention conform to formula I: 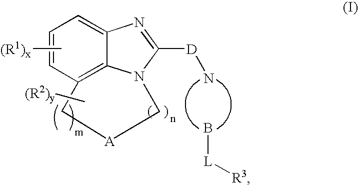
wherein R¹, R², R³, A, D, B, m, n, x, and y are defined as described above.

In one embodiment, variable m is 0 and variable n is 2. In further embodiments A is selected from the group consisting of CH₂ and O.

In other embodiments, D is —(CR⁵R⁶)_{z}—. In still other embodiments, z is preferably 1. In other embodiments, each of R⁵ and R⁶is H.

In still other embodiments, 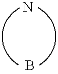
represents a piperidine ring.

Further embodiments provide for R³ being a 5- to 7-membered ring that is optionally substituted by 1-3 R¹groups. The ring may contain one or more heteroatoms independently selected from the group consisting of N, O and S. In certain embodiments, R³ is phenyl that is optionally substituted by 1-3 R¹.

Additional embodiments provide for compounds of formula I wherein m is 0, n is 2, and A is CH₂ or O. In these embodiments, R¹ is selected from the group consisting of H, F, Cl, Br, I, nitro, C₁₋₆-alkyl, C₁₋₆-alkylhalo, C₁₋₆-alkylhalo, OC₁₋₆-alkylhalo, aryl, C₁₋₆-alkylenearyl, and OC₁₋₆-alkylenearyl, while R² is selected from H and C₁₋₆-alkyl.

When compounds of the present invention contain one or more chiral centers, those compounds may exist in and be isolated as enantiomeric or diastereomeric forms, or as a racemic mixture. The present invention includes any possible enantiomers, diastereomers, racemates or mixtures thereof, of a compound of formula I. The optically active forms of the compound of the invention may be prepared, for example, by chiral chromatographic separation of a racemate, by synthesis from optically active starting materials or by asymmetric synthesis based on the procedures described thereafter.

Certain compounds of the present invention may exist as geometrical isomers, for example, E and Z isomers of alkenes. The present invention includes any geometrical isomer of a compound of formula I. By the same token, the present invention encompasses tautomers of the compounds of formula I.

In addition, certain compounds of the present invention may exist in solvated forms, such as hydrated, as well as in unsolvated forms. Thus, the present invention encompasses all such solvated forms of the compounds of formula I.

Also within the scope of the invention are salts of the compounds of formula I. Generally, pharmaceutically acceptable salts of compounds of the present invention are obtained using standard procedures well known in the art, for example, by reacting a sufficiently basic compound, for example an alkyl amine with a suitable acid, for example, HCl or acetic acid, to afford a physiologically acceptable anion. It is possible as well to make a corresponding alkali metal (such as sodium, potassium, or lithium) or an alkaline earth metal (such as a calcium) salt by treating a compound of the present invention having a suitably acidic proton, such as a carboxylic acid or a phenol with one equivalent of an alkali metal or alkaline earth metal hydroxide or alkoxide (such as the ethoxide or methoxide), or a suitably basic organic amine (such as choline or meglumine) in an aqueous medium, followed by conventional purification techniques.

In one embodiment of the present invention, the compound of formula I may be converted to a pharmaceutically acceptable salt or solvate thereof, in particular an acid addition salt such as a hydrochloride, hydrobromide, phosphate, acetate, fumarate, maleate, tartrate, citrate, methanesulphonate or p-toluenesulphonate.

Specific examples of the present invention include the following compounds, their pharmaceutically acceptable salts, hydrates, solvates, optical isomers, and combinations thereof:

| Example No./Structure | Name |
|---|---|
| 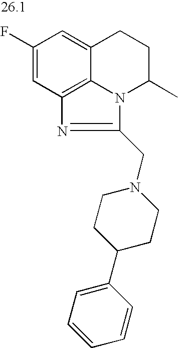 | 8-fluoro-4-meth- yl-2-[(4-phenyl- piperidin-1-yl)meth- yl]-5,6-dihydro-4H-imi- dazo[4,5,1-ij]quino- line |
| 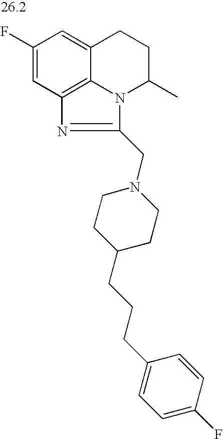 | 8-fluoro-4-meth- yl-2-{4-[3-(4-fluorophenyl)pro- pyl]piperidin-1-yl- methyl}-5,6-dihydro-4H-imi- dazo[4,5,1-ij]quino- line |
| 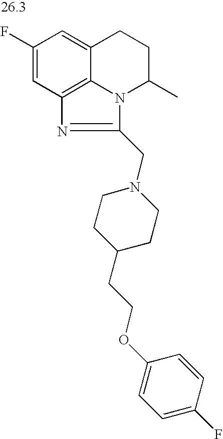 | 8-fluoro-4-meth- yl-2-{4-[2-(4-fluoro- phenoxy)eth- yl]piperidin-1-yl- methyl}-5,6-dihydro-4H-imi- dazo[4,5,1-ij]quinoline |
| 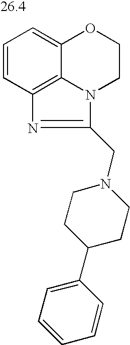 | 2-[(4-phenylpiperidin-1-yl)meth- yl]-4,5-dihydro- imidazo[1,5,4-de][1,4]benz- oxazine |
| 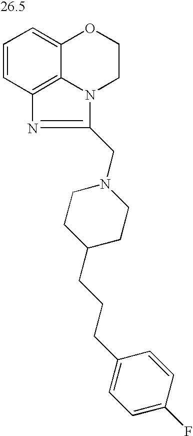 | 2-{4-[3-(4-fluoro- phenyl)propyl]pipe- ridin-1-ylmethyl}-4,5-di- hydroimi- dazo[1,5,4-de][1,4]benz- oxazine |
| 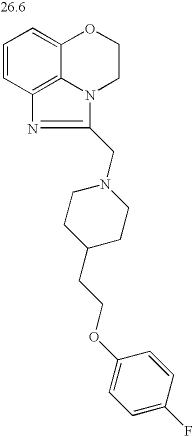 | 2-{4-[2-(4-fluoro- phenoxy)eth- yl]piperidin-1-yl- methyl}-4,5-dihydro- imidazo[1,5,4-de][1,4]benz- oxazine |
| 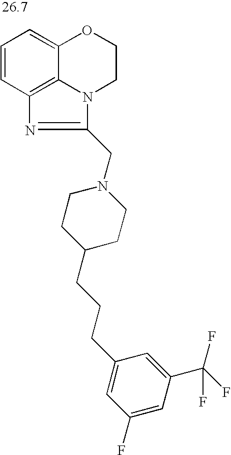 | 2-{4-[3-(3-fluoro-5-(tri- fluoromethyl)-phe- nyl)propyl]pipe- ridin-1-ylmethyl}-4,5-di- hydroimi- dazo[1,5,4-de][1,4]benz- oxazine |
| 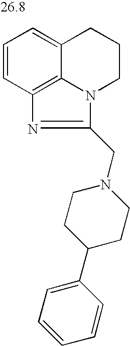 | 2-[(4-phenylpiperidin-1-yl)meth- yl]-5,6-dihydro-4H-imi- dazo[4,5,1-ij]quinoline |
| 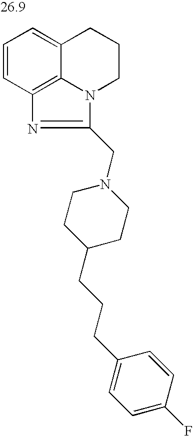 | 2-{4-[3-(4-fluoro- phenyl)propyl]pipe- ridin-1-ylmethyl}-5,6-di- hydro-4H-imi- dazo[4,5,1-ij]quinoline |
| 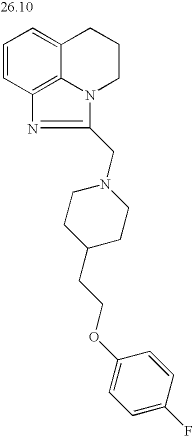 | 2-{4-[2-(4-fluoro- phenoxy)eth- yl]piperidin-1-ylmeth- yl}-5,6-dihydro-4H-imi- dazo[4,5,1-ij]quinoline |
| 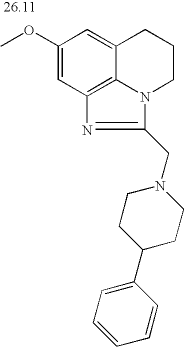 | 8-methoxy-2-[(4-phenyl- piperidin-1-yl)meth- yl]-5,6-dihydro-4H-imi- dazo[4,5,1-ij]quinoline |
| 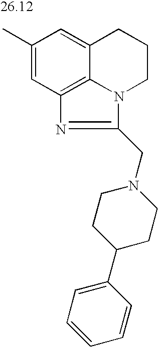 | 8-methyl-2-[(4-phenyl- piperidin-1-yl)meth- yl]-5,6-dihydro-4H-imi- dazo[4,5,1-ij]quinoline |
| 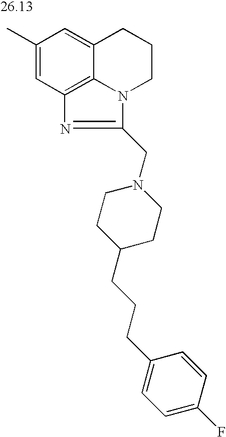 | 8-methyl-2-{4-[3-(4-fluoro- phenyl)propyl]pipe- ridin-1-ylmethyl}-5,6-di- hydro-4H-imi- dazo[4,5,1-ij]quino- line |
| 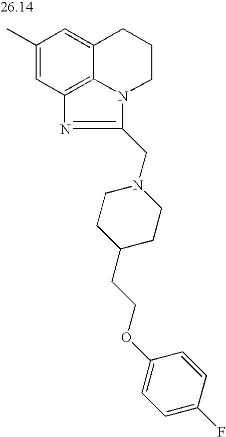 | 8-methyl-2-{4-[2-(4-fluoro- phenoxy)eth- yl]piperidin-1-yl- methyl}-5,6-dihydro-4H-imi- dazo[4,5,1-ij]quinoline |
| 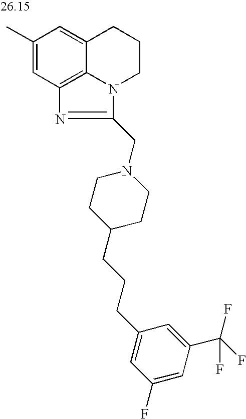 | 8-methyl-2-{4-[3-(3-fluoro-5(tri- fluoromethyl)-phenyl)pro- pyl]piperidin-1-yl- methyl}-5,6-dihydro-4H-imi- dazo[4,5,1-ij]quinoline |
| 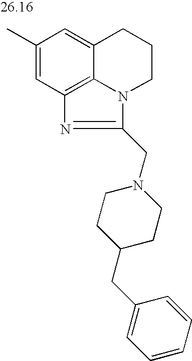 | 8-methyl-2-[(4-benzyl- piperidin-1-yl)meth- yl]-5,6-dihydro-4H-imi- dazo[4,5,1-ij]quinoline |
| 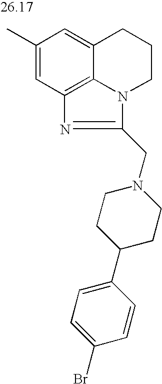 | 8-methyl-2-{[4-(4-bromo- phenyl)piperidin-1-yl]meth- yl}-5,6-di- hydro-4H-imi- dazo[4,5,1-ij]quinoline |
| 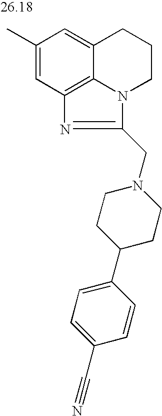 | 8-methyl-2-{[4-(4-cyano- phenyl)piperidin-1-yl]meth- yl}-5,6-dihydro-4H-imi- dazo[4,5,1-ij]quinoline |
| 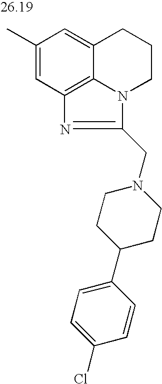 | 8-methyl-2-{[4-(4-chloro- phenyl)piperidin-1-yl]meth- yl}-5,6-di- hydro-4H-imi- dazo[4,5,1-ij]quino- line |
| 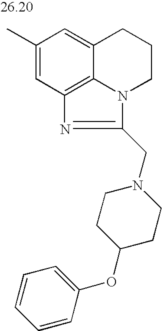 | 8-methyl-2-[(4-phenoxy- piperidin-1-yl)meth- yl]-5,6-dihydro-4H-imi- dazo[4,5,1-ij]quinoline |
| 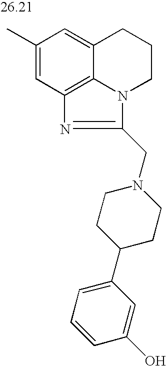 | 8-methyl-2-{[4-(3-hydroxy- phenyl)piperidin-1-yl]meth- yl}-5,6-dihydro-4H-imi- dazo[4,5,1-ij]quinoline |
| 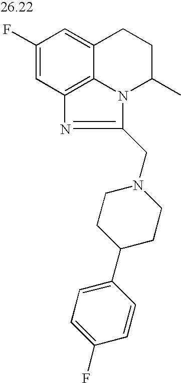 | 8-fluoro-4-meth- yl-2-[4-(4-fluoro- phenyl)-pipe- ridin-1-ylmethyl]-4-meth- yl-5,6-dihydro-4H-imi- dazo[4,5,1-ij]quinoline |
| 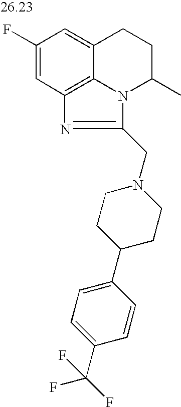 | 8-fluoro-4-methyl-2-[4-(4-tri- fluoromethylphenyl)-pipe- ridin-1-ylmethyl]-5,6-di- hydro-4H-imi- dazo[4,5,1-ij]quinoline |
| 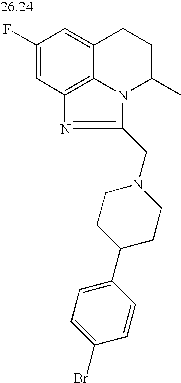 | 8-fluoro-4-meth- yl-2-[4-(4-bromo- phenyl)-pipe- ridin-1-ylmethyl]-5,6-di- hydro-4H-imi- dazo[4,5,1-ij]quinoline |
| 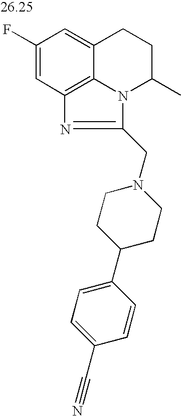 | 8-fluoro-4-meth- yl-2-[4-(4-cyano- phenyl)-pipe- ridin-1-ylmethyl]-5,6-di- hydro-4H-imi- dazo[4,5,1-ij]quinoline |
| 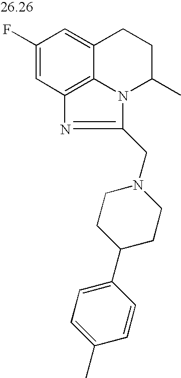 | 8-fluoro-4-meth- yl-2-[4-(4-methyl- phenyl)-pipe- ridin-1-ylmethyl]-5,6-di- hydro-4H-imi- dazo[4,5,1-ij]quinoline |
| 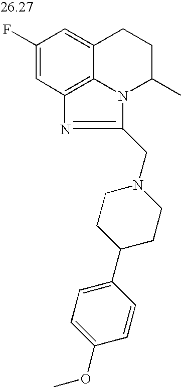 | 8-fluoro-4-meth- yl-2-[4-(4-meth- oxyphenyl)-pipe- ridin-1-ylmethyl]-5,6-di- hydro-4H-imi- dazo[4,5,1-ij]quinoline |
| 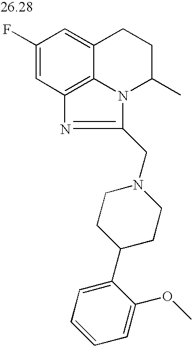 | 8-fluoro-4-meth- yl-2-[4-(2-meth- oxyphenyl)-pipe- ridin-1-ylmethyl]-5,6-di- hydro-4H-imi- dazo[4,5,1-ij]quinoline |
| 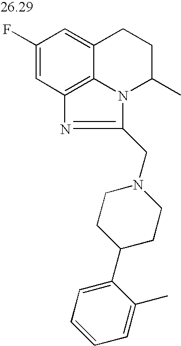 | 8-fluoro-4-meth- yl-2-[4-(2-meth- ylphenyl)-pipe- ridin-1-ylmethyl]-5,6-di- hydro-4H-imi- dazo[4,5,1-ij]quinoline |
| 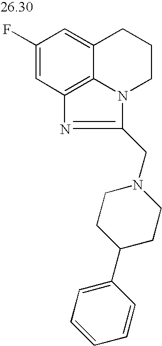 | 8-fluoro-2-[(4-phenyl- piperidin-1-yl)meth- yl]-5,6-dihydro-4H-imi- dazo[4,5,1-ij]quinoline |
| 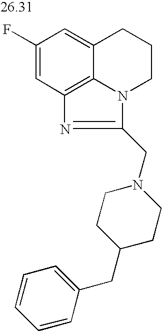 | 8-fluoro-2-[(4-benzyl- piperidin-1-yl)meth- yl]-5,6-dihydro-4H-imi- dazo[4,5,1-ij]quinoline |
| 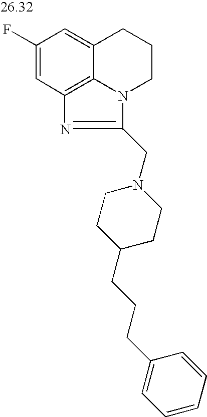 | 8-fluoro-2-[4-(3-phenyl propyl)piperidin-1-yl- methyl]-5,6-dihydro-4H-imi- dazo[4,5,1-ij]quinoline |
| 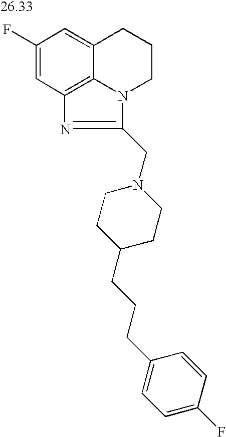 | 8-fluoro-2-{4-[3-(4-fluoro- phenyl)pro- pyl]piperidin-1-yl- methyl}-5,6-dihydro-4H-imi- dazo[4,5,1-ij]quinoline |
| 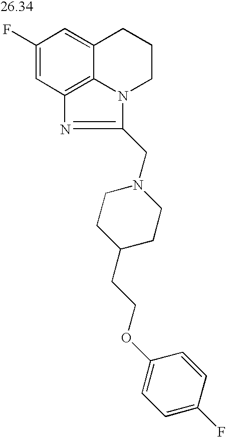 | 8-fluoro-2-{4-[2-(4-fluoro- phenoxy)eth- yl]piperidin-1-yl- methyl}-5,6-dihydro-4H-imi- dazo[4,5,1-ij]quinoline |
| 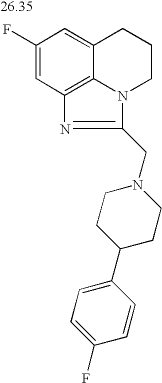 | 8-fluoro-2-{[4-(4-fluoro- phenyl)piperidin-1-yl]meth- yl}-5,6-di- hydro-4H-imi- dazo[4,5,1-ij]quinoline |
| 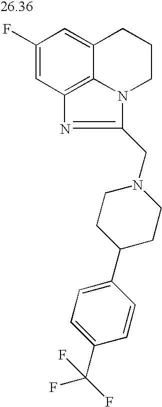 | 8-fluoro-2-{[4-(4-tri- fluoromethyl- phenyl)piperidin-1-yl]meth- yl}-5,6-dihydro-4H-imi- dazo[4,5,1-ij]quinoline |
| 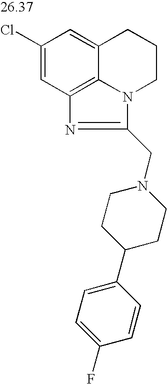 | 8-chloro-2-{[4-(4-fluoro- phenyl)piperidin-1-yl]meth- yl}-5,6-di- hydro-4H-imi- dazo[4,5,1-ij]quinoline |
| 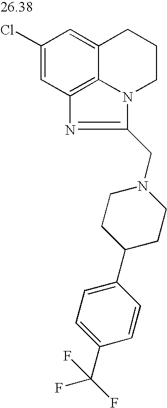 | 8-chloro-2-{[4-(4-tri- fluoromethyl- phenyl)piperidin-1-yl]meth- yl}-5,6-dihydro-4H-imi- dazo[4,5,1-ij]quinoline |
| 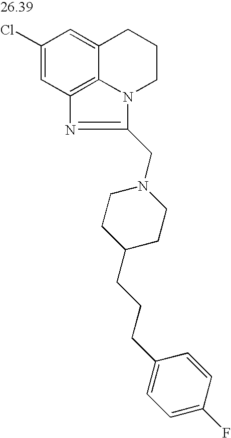 | 8-chloro-2-{4-[3-(4-fluoro- phenyl)pro- pyl]piperidin-1-yl- methyl}-5,6-dihydro-4H-imi- dazo[4,5,1-ij]quinoline |
| 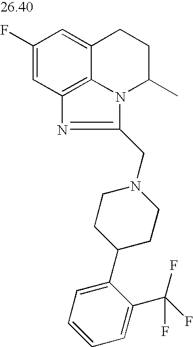 | 8-fluoro-4-methyl-2-[4-(2-tri- fluoromethylphenyl)-pipe- ridin-1-ylmethyl]-5,6-di- hydro-4H-imi- dazo[4,5,1-ij]quino- line |
| 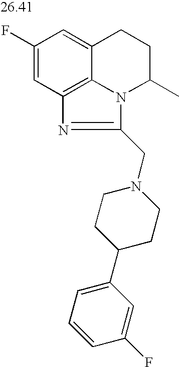 | 8-fluoro-4-meth- yl-2-[4-(3-fluoro- phenyl)-pipe- ridin-1-ylmethyl]-5,6-di- hydro-4H-imi- dazo[4,5,1-ij]quinoline |
| 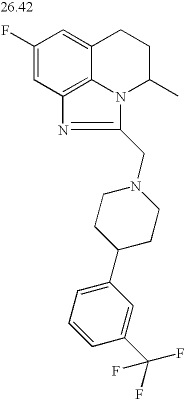 | 8-fluoro-4-methyl-2-[4-(3-tri- fluoromethylphenyl)-pipe- ridin-1-ylmethyl]-5,6-di- hydro-4H-imi- dazo[4,5,1-ij]quinoline |
| 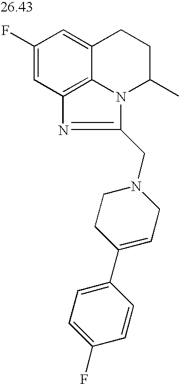 | 8-fluoro-4-meth- yl-2-{[4-(4-fluoro- phenyl)-3,6-di- hydropyridin-1(2H)-yl]meth- yl}-5,6-di- hydro-4H-imi- dazo[4,5,1-ij]quinoline |
| 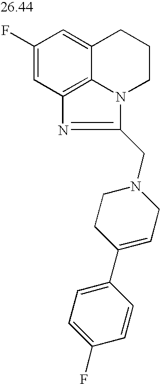 | 8-fluoro-2-{[4-(4-fluoro- phenyl)-3,6-di- hydropyridin-1(2H)-yl]meth- yl}-5,6-dihydro-4H-imi- dazo[4,5,1-ij]quinoline |
| 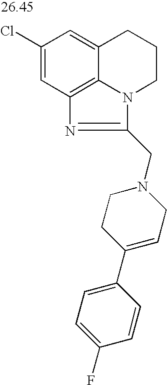 | 8-chloro-2-{[4-(4-fluoro- phenyl)-3,6-di- hydropyridin-1(2H)-yl]meth- yl}-5,6-di- hydro-4H-imi- dazo[4,5,1-ij]quinoline |
| 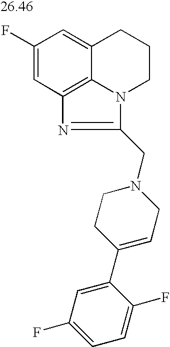 | 8-fluoro-4-methyl-2-{[4-(2,5-di- fluorophenyl)-3,6-di- hydropyridin-1(2H)-yl]meth- yl}-5,6-dihydro-4H-imi- dazo[4,5,1-ij]quinoline |
| 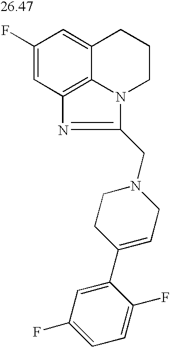 | 8-fluoro-2-{[4-(2,5-di- fluorophenyl)-3,6-di- hydropyridin-1(2H)-yl]meth- yl}-5,6-di- hydro-4H-imi- dazo[4,5,1-ij]quinoline |
| 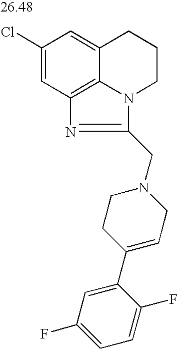 | 8-chloro-2-{[4-(2,5-di- fluorophenyl)-3,6-di- hydropyridin-1(2H)-yl]meth- yl}-5,6-dihydro-4H-imi- dazo[4,5,1-ij]quinoline |
| 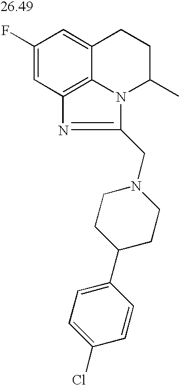 | 8-fluoro-4-meth- yl-2-[4-(4-chloro- phenyl)-pipe- ridin-1-ylmethyl]-5,6-di- hydro-4H-imi- dazo[4,5,1-ij]quinoline |
| 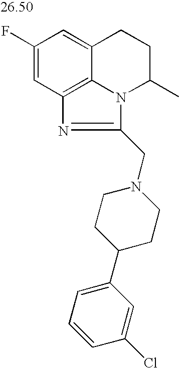 | 8-fluoro-4-meth- yl-2-[4-(3-chloro- phenyl)-pipe- ridin-1-ylmethyl]-5,6-di- hydro-4H-imi- dazo[4,5,1-ij]quinoline |
| 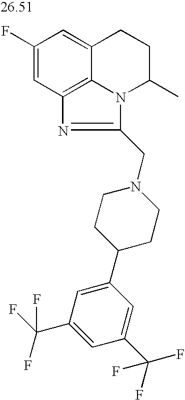 | 8-fluoro-4-meth- yl-2-[4-(3,5-bis(tri- fluoromethyl)-phe- nyl)-piperidin-1-yl- methyl]-5,6-dihydro-4H-imi- dazo[4,5,1-ij]quinoline |
| 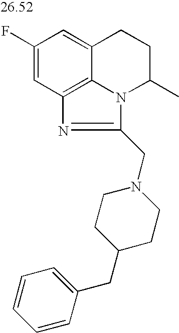 | 8-fluoro-4-meth- yl-2-[4-benzyl- phenyl)-piperidin-1-yl- methyl]-5,6-dihydro-4H-imi- dazo[4,5,1-ij]quinoline |
| 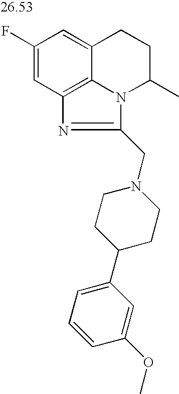 | 8-fluoro-4-methyl-2-[4-(3-meth- oxyphenyl)-pipe- ridin-1-ylmethyl]-5,6-di- hydro-4H-imi- dazo[4,5,1-ij]quinoline |
| 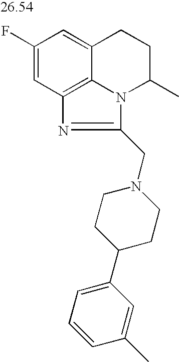 | 8-fluoro-4-methyl-2-[4-(3-meth- ylphenyl)-pipe- ridin-1-ylmethyl]-5,6-di- hydro-4H-imi- dazo[4,5,1-ij]quinoline |
| 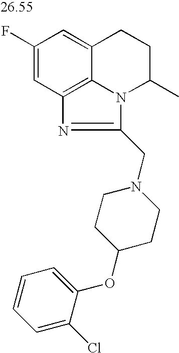 | 8-fluoro-4-meth- yl-2-[4-(2-chloro- phenoxy)-pipe- ridin-1-ylmethyl]-5,6-di- hydro-4H-imi- dazo[4,5,1-ij]quinoline |
| 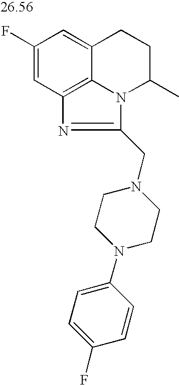 | 8-fluoro-4-meth- yl-2-{[4(4-fluoro- phenyl)piperizin-1-yl]meth- yl}-5,6-dihydro-4H-imi- dazo[4,5,1-ij]quinoline |
| 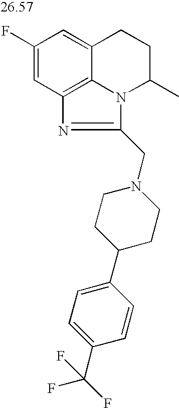 | 8-fluoro-4-methyl-2-{[4(4-tri- fluoromethyl-phenyl)pipe- rizin-1-yl]meth- yl}-5,6-di- hydro-4H-imi- dazo[4,5,1-ij]quinoline |
| 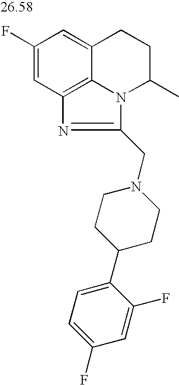 | 8-fluoro-4-meth- yl-2-{[4(2,4-di- fluorophenyl)pipe- rizin-1-yl]meth- yl}-5,6-di- hydro-4H-imi- dazo[4,5,1-ij]quinoline |
| 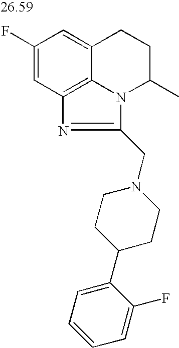 | 8-fluoro-4-meth- yl-2-[4-(2-fluoro- phenyl)-pipe- ridin-1-ylmethyl]-5,6-di- hydro-4H-imi- dazo[4,5,1-ij]quinoline |
| 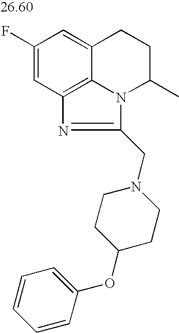 | 8-fluoro-4-methyl-2-[4-phenoxy- piperidin-1-yl- methyl]-5,6-dihydro-4H-imi- dazo[4,5,1-ij]quinoline |
| 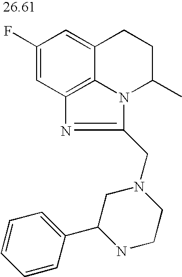 | 8-fluoro-4-methyl-2-[3-phenyl- piperazin-1-yl- methyl]-5,6-dihydro-4H-imi- dazo[4,5,1-ij]quinoline |
| 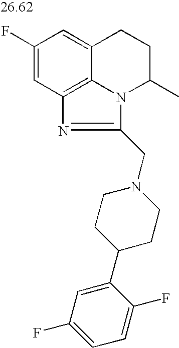 | 8-fluoro-4-methyl-2-{[4-(2,5-di- fluorophenyl)pipe- ridin-1-yl]meth- yl}-5,6-di- hydro-4H-imi- dazo[4,5,1-ij]quinoline |
| 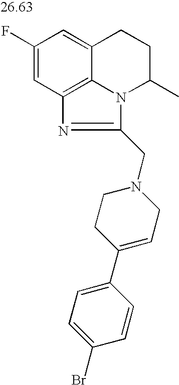 | 8-fluoro-4-meth- yl-2-{[4-(4-bromo- phenyl)-3,6-di- hydropyridin-1(2H)-yl]meth- yl}-5,6-di- hydro-4H-imi- dazo[4,5,1-ij]quinoline |
| 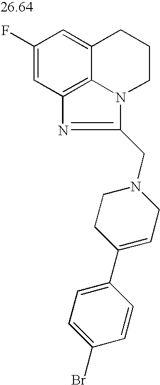 | 8-fluoro-2-{[4-(4-bromo- phenyl)-3,6-di- hydropyridin-1(2H)-yl]meth- yl}-5,6-dihydro-4H-imi- dazo[4,5,1-ij]quinoline |
| 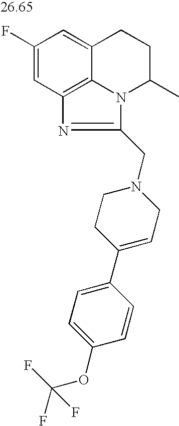 | 8-fluoro-4-methyl-2-{[4-(4-tri- fluoromethoxyphenyl)-3,6-di- hydropyridin-1(2H)-yl]meth- yl}-5,6-di- hydro-4H-imi- dazo[4,5,1-ij]quinoline |
| 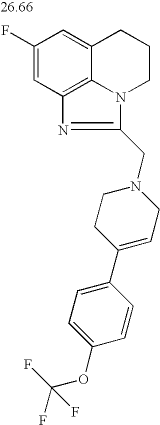 | 8-fluoro-2-{[4-(4-tri- fluoromethoxyphenyl)-3,6-di- hydropyridin-1(2H)-yl]meth- yl}-5,6-di- hydro-4H-imi- dazo[4,5,1-ij]quinoline |
| 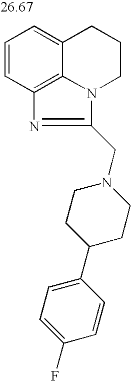 | 2-[(4-(4-fluoro- phenyl)piperidin-1-yl)meth- yl]-5,6-di- hydro-4H-imi- dazo[4,5,1-ij]quinoline |
| 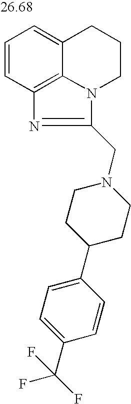 | 2-[(4-(4-tri- fluoromethylphenyl)pipe- ridin-1-yl)methyl]-5,6-di- hydro-4H-imi- dazo[4,5,1-ij]quinoline |
| 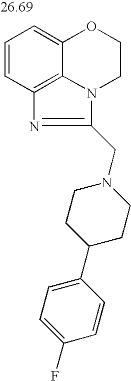 | 2-[(4-(4-fluoro- phenylpiperidin-1-yl)meth- yl]-4,5-di- hydroimi- dazo[1,5,4-de][1,4]benz- oxazine |
| 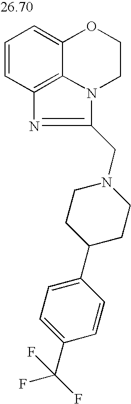 | 2-[(4-(4-tri- fluoromethylphenylpipe- ridin-1-yl)methyl]-4,5-di- hydroimi- dazo[1,5,4-de][1,4]benz- oxazine |
| 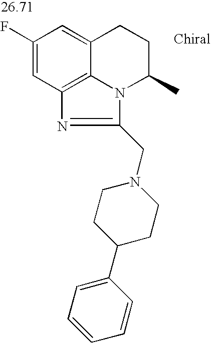 | [4R]-8-fluoro-4-meth- yl-2-[(4-phenyl- piperidin-1-yl)meth- yl]-5,6-di- hydro-4H-imi- dazo[4,5,1-ij]quinoline |
| 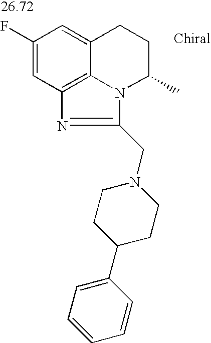 | [4S]-8-fluoro-4-meth- yl-2-[(4-phenyl- piperidin-1-yl)meth- yl]-5,6-dihydro-4H-imi- dazo[4,5,1-ij]quinoline |
| 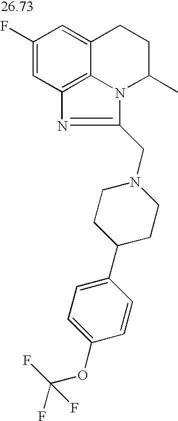 | 8-fluoro-4-methyl-2-{[4-(4-tri- fluoromethoxyphenyl)pipe- ridin-1-yl]meth- yl}-5,6-di- hydro-4H-imi- dazo[4,5,1-ij]quinoline |
| 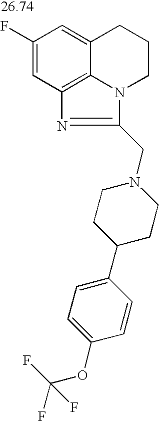 | 8-fluoro-2-{[4-(4-tri- fluoromethoxyphenyl)pipe- ridin-1-yl]methyl}-5,6-di- hydro-4H-imi- dazo[4,5,1-ij]quinoline |
| 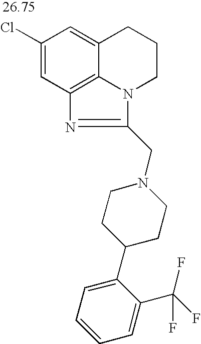 | 8-chloro-2-[(4-(2-tri- fluoromethylphenyl)pipe- ridin-1-yl)methyl]-5,6-di- hydro-4H-imi- dazo[4,5,1-ij]quinoline |
| 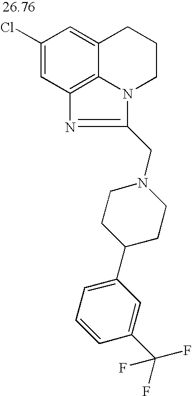 | 8-chloro-2-[(4-(3-tri- fluoromethylphenyl)pipe- ridin-1-yl)methyl]-5,6-di- hydro-4H-imi- dazo[4,5,1-ij]quinoline |
| 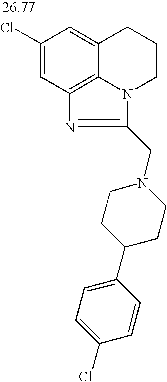 | 8-chloro-2-[(4-(4-chloro- phenyl)piperidin-1-yl)meth- yl]-5,6-di- hydro-4H-imi- dazo[4,5,1-ij]quinoline |
| 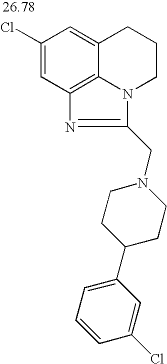 | 8-chloro-2-[(4-(3-chloro- phenyl)piperidin-1-yl)meth- yl]-5,6-di- hydro-4H-imi- dazo[4,5,1-ij]quinoline |
| 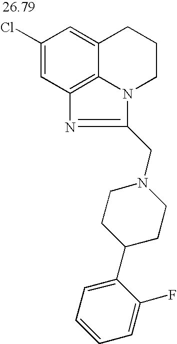 | 8-chloro-2-[(4-(2-fluoro- phenyl)piperidin-1-yl)meth- yl]-5,6-di- hydro-4H-imi- dazo[4,5,1-ij]quinoline |
| 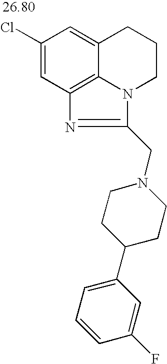 | 8-chloro-2-[(4-(3-fluoro- phenyl)piperidin-1-yl)meth- yl]-5,6-dihydro-4H-imi- dazo[4,5,1-ij]quinoline |
| 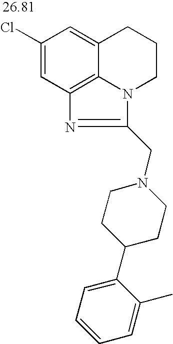 | 8-chloro-2-[(4-(2-methyl- phenyl)piperidin-1-yl)meth- yl]-5,6-di- hydro-4H-imi- dazo[4,5,1-ij]quinoline |
| 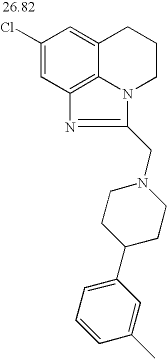 | 8-chloro-2-[(4-(3-methyl- phenyl)piperidin-1-yl)meth- yl]-5,6-di- hydro-4H-imi- dazo[4,5,1-ij]quinoline |
| 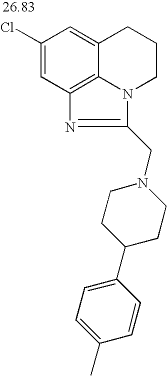 | 8-chloro-2-[(4-(4-methyl- phenyl)piperidin-1-yl)meth- yl]-5,6-di- hydro-4H-imi- dazo[4,5,1-ij]quinoline |
| 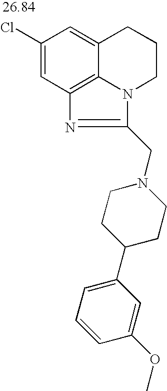 | 8-chloro-2-[(4-(3-meth- oxyphenyl)piperidin-1-yl)meth- yl]-5,6-di- hydro-4H-imi- dazo[4,5,1-ij]quinoline |
| 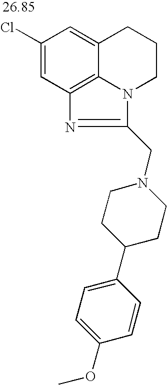 | 8-chloro-2-[(4-(4-meth- oxyphenyl)piperidin-1-yl)meth- yl]-5,6-di- hydro-4H-imi- dazo[4,5,1-ij]quinoline |
| 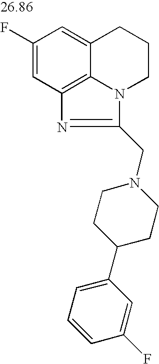 | 8-fluoro-2-{[4-(3-fluoro- phenyl)piperidin-1-yl]meth- yl}-5,6-dihydro-4H-imi- dazo[4,5,1-ij]quinoline |
| 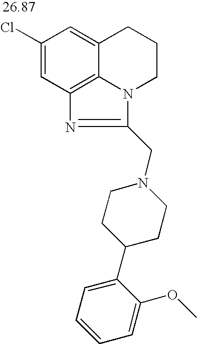 | 8-chloro-2-[(4-(2-meth- oxyphenyl)piperidin-1-yl)meth- yl]-5,6-di- hydro-4H-imi- dazo[4,5,1-ij]quino- line |
| 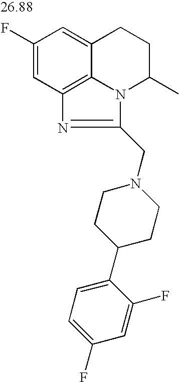 | 8-fluoro-4-methyl-2-[4-(2,4-di- fluoro-phenyl)-pipe- ridin-1-ylmethyl]-5,6-di- hydro-4H-imi- dazo[4,5,1-ij]quinoline |
| 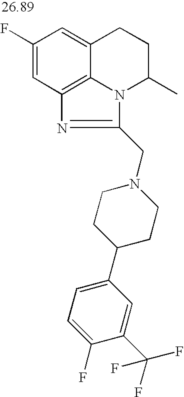 | 8-fluoro-4-meth- yl-2-[4-(4-fluoro-3-tri- fluoromethyl- phenyl)-piperidin-1-yl- methyl]-5,6-dihydro-4H-imi- dazo[4,5,1-ij]quino- line |
| 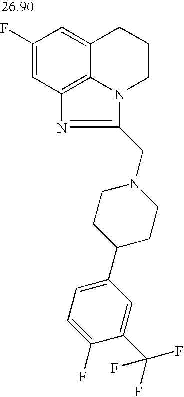 | 8-fluoro-2-[4-(4-fluoro-3-tri- fluoromethyl- phenyl)-piperidin-1-yl- methyl]-5,6-dihydro-4H-imi- dazo[4,5,1-ij]quino- line |
| 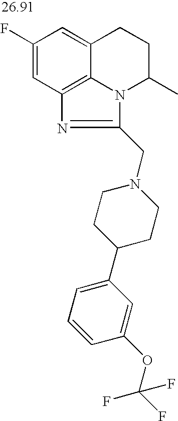 | 8-fluoro-4-methyl-2-{[4-(3-tri- fluoromethoxyphenyl)pipe- ridin-1-yl]meth- yl}-5,6-di- hydro-4H-imi- dazo[4,5,1-ij]quino- line |
| 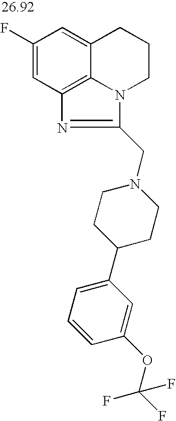 | 8-fluoro-2-{[4-(3-tri- fluoromethoxyphenyl)pipe- ridin-1-yl]methyl}-5,6-di- hydro-4H-imi- dazo[4,5,1-ij]quino- line |
| 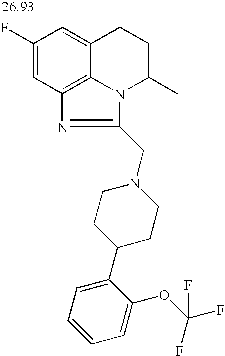 | 8-fluoro-4-methyl-2-{[4-(2-tri- fluoromethoxyphenyl)pipe- ridin-1-yl]meth- yl}-5,6-di- hydro-4H-imi- dao[4,5,1-ij]quinoline |
| 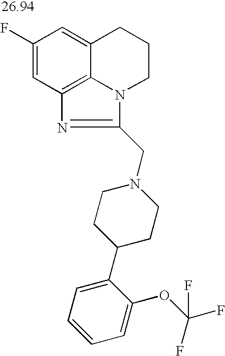 | 8-fluoro-2-{[4-(2-tri- fluoromethoxy phenyl)pipe- ridin-1-yl]methyl}-5,6-di- hydro-4H-imi- dazo[4,5,1-ij]quinoline |
| 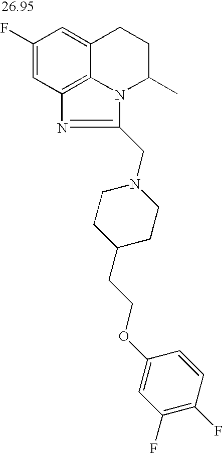 | 8-fluoro-4-meth- yl-2-{4-[2-(3,4-di- fluorophenoxy)ethyl]pipe- ridin-1-ylmethyl}-5,6-di- hydro-4H-imi- dazo[4,5,1-ij]quinoline |
| 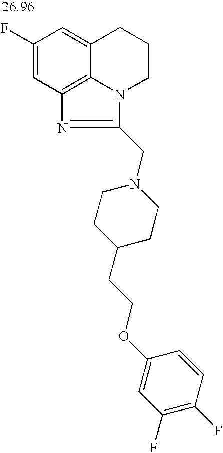 | 8-fluoro-2-{4-[2-(3,4-di- fluorophenoxy)ethyl]pipe- ridin-1-ylmethyl}-5,6-di- hydro-4H-imi- dazo[4,5,1-ij]quinoline |
| 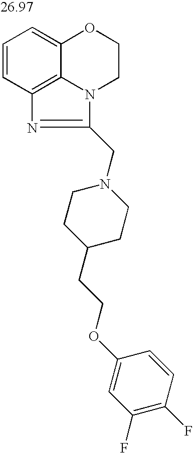 | 2-{4-[2-(3,4-di- fluorophenoxy)ethyl]pipe- ridin-1-ylmethyl}-4,5-di- hydroimidazo[1,5,4-de][1,4]ben- zoxazine |
| 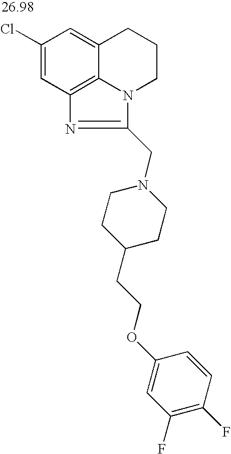 | 8-chloro-2-{4-[2-(3,4-di- fluorophenoxy)ethyl]pipe- ridin-1-ylmethyl}-5,6-di- hydro-4H-imi- dazo[4,5,1-ij]quinoline |
| 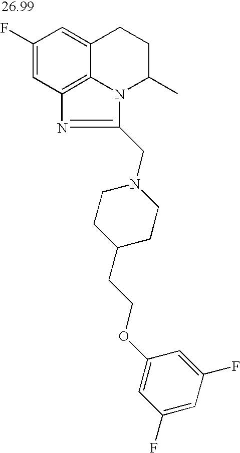 | 8-fluoro-4-meth- yl-2-{4-[2-(3,5-di- fluorophenoxy)ethyl]pipe- ridin-1-ylmethyl}-5,6-di- hydro-4H-imi- dazo[4,5,1-ij]quinoline |
| 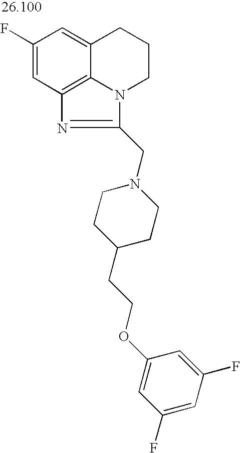 | 8-fluoro-2-{4-[2-(3,5-di- fluorophenoxy)ethyl]pipe- ridin-1-ylmethyl}-5,6-di- hydro-4H-imi- dazo[4,5,1-ij]quinoline |
| 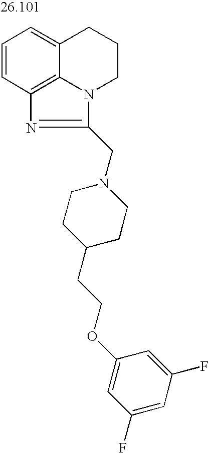 | 2-{4-[2-(3,5-di- fluorophenoxy)ethyl]pipe- ridin-1-ylmethyl}-4,5-di- hydroimi- dazo[1,5,4-de][1,4]benz- oxazine |
| 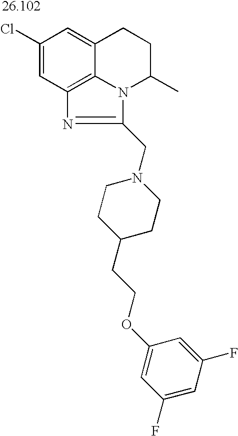 | 8-chloro-2-{4-[2-(3,5-di- fluorophenoxy)ethyl]pipe- ridin-1-ylmethyl}-5,6-di- hydro-4H-imi- dazo[4,5,1-ij]quinoline |
| 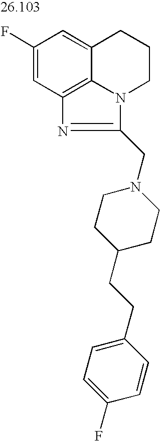 | 8-fluoro-2-{4-[2-(4-fluoro- phenyl)eth- yl]piperidin-1-yl- methyl}-5,6-dihydro-4H-imi- dazo[4,5,1-ij]quinoline |
| 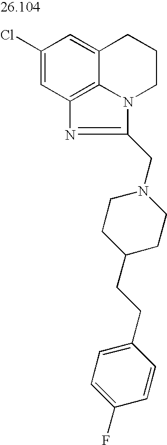 | 8-chloro-2-{4-[2-(4-fluoro- phenyl)eth- yl]piperidin-1-yl- methyl}-5,6-dihydro-4H-imi- dazo[4,5,1-ij]quinoline |
| 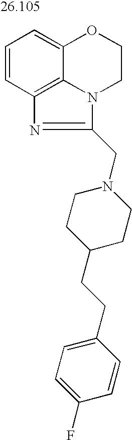 | 2-{4-[2-(4-fluoro- phenyl)ethyl]pipe- ridin-1-ylmethyl}-4,5-di- hydroimi- dazo[1,5,4-de][1,4]benz- oxazine |
| 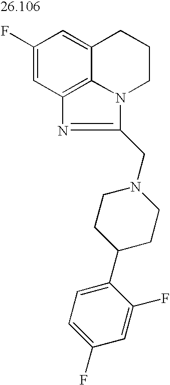 | 8-fluoro-2-{[4-(2,4-di- fluorophenyl)pipe- ridin-1-yl]meth- yl}-5,6-di- hydro-4H-imi- dazo[4,5,1-ij]quinoline |
| 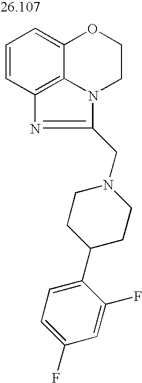 | 2-[(4-2,4-di- fluorophenyl piperidin-1-yl)methyl]-4,5-di- hydroimi- dazo[1,5,4-de][1,4]benz- oxazine |
| 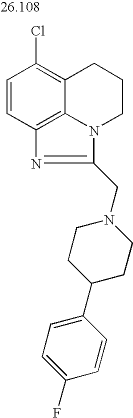 | 7-Chloro-2-[4-(4-fluoro- phenyl)-pipe- ridin-1-ylmethyl]-5,6-di- hydro-4H-imi- dazo[4,5,1-ij]quinoline |
| 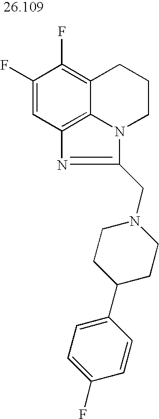 | 7,8-Difluoro-2-[4-(4-fluoro- phenyl)-pipe- ridin-1-ylmethyl]-5,6-di- hydro-4H-imi- dazo[4,5,1-ij]quinoline |
| 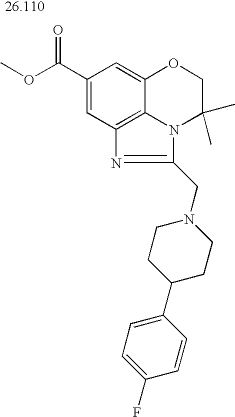 | 2-[4-(4-Fluoro-phenyl)-pipe- ridin-1-yl- methyl]-3,3-dimethyl-3,4-di- hydro-5-oxa-1,2a-diaza- acenaphthylene-7-carboxylic acid methyl ester |
| 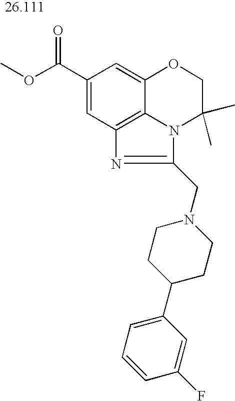 | 2-[4-(3-Fluoro-phenyl)-pipe- ridin-1-yl- methyl]-3,3-dimethyl-3,4-di- hydro-5-oxa-1,2a-diaza- acenaphthylene-7-carboxylic acid methyl ester |
| 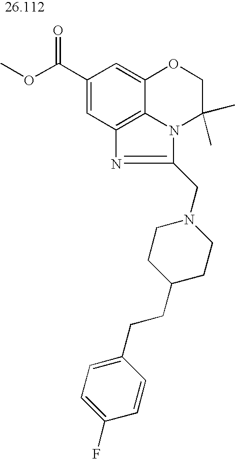 | 2-{4-[2-(4-Fluoro- phenyl)-ethyl]-pipe- ridin-1-ylmethyl}-3,3-di- methyl-3,4-di- hydro-5-oxa-1,2a-diaza- acenaphthylene-7-carboxylic acid methyl ester |
| 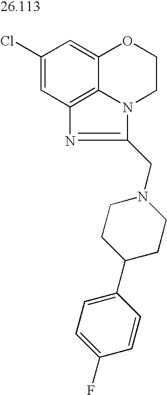 | 7-Chloro-2-[4-(4-fluoro- phenyl)-pipe- ridin-1-ylmethyl]-3,4-di- hydro-5-oxa-1,2a-di- aza-acenaphthylene |
| 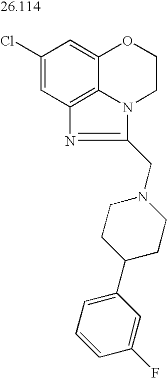 | 7-Chloro-2-[4-(3-fluoro- phenyl)-pipe- ridin-1-ylmethyl]-3,4-di- hydro-5-oxa-1,2a-di- aza-acenaphthylene |
| 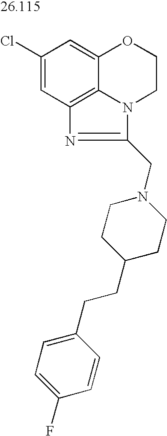 | 7-Chloro-2-{4-[2-(4-fluoro- phenyl)-eth- yl]-piperidin-1-yl- methyl}-3,4-di- hydro-5-oxa-1,2a-di- aza-acenaphthylene |
| 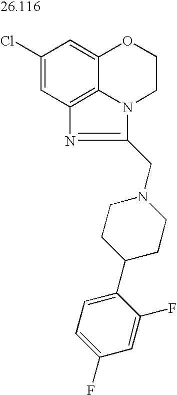 | 7-Chloro-2-[4-(2,4-di- fluoro-phenyl)-pipe- ridin-1-ylmethyl]-3,4-di- hydro-5-oxa-1,2a-di- aza-acenaphthylene |
| 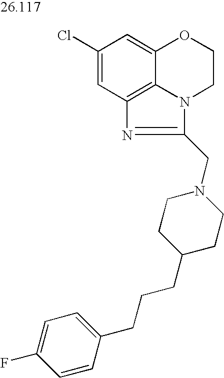 | 7-Chloro-2-{4-[3-(4-fluoro- phenyl)-pro- pyl]-piperidin-1-yl- methyl}-3,4-di- hydro-5-oxa-1,2a-diaza- acenaphthylene |
| 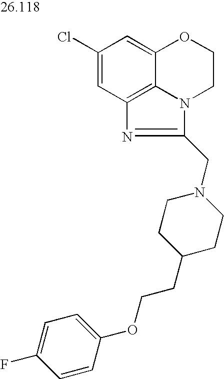 | 7-Chloro-2-{4-[2-(4-fluoro- phenoxy)-eth- yl]-piperidin-1-yl- methyl}-3,4-di- hydro-5-oxa-1,2a-diaza- acenaphthylene |
| 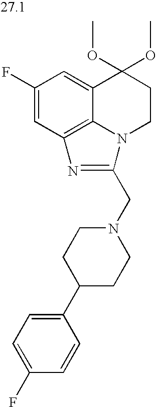 | 8-Fluoro-2-[4-(4-fluoro- phenyl)-pipe- ridin-1-ylmethyl]-6,6-di- methoxy-5,6-di- hydro-4H-imi- dazo[4,5,1-ij]quinoline |
| 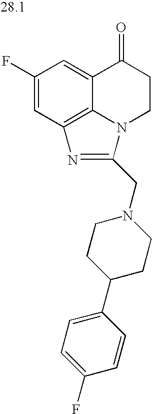 | 8-Fluoro-2-[4-(4-fluoro- phenyl)-piperidin-1-yl- methyl]-4,5,9a,9b-tetra- hydroimidazo[4,5,1-ij]quino- lin-6-one |
| 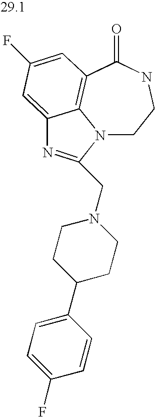 | 4-Fluoro-1-[4-(4-fluoro- phenyl)-pipe- ridin-1-ylmethyl]-8,9-di- hydro-7H-2,7,9a-triaza- benzo[cd]azulen-6-one |
| 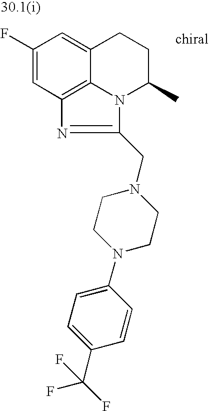 | [4R]-8-Fluoro-4-meth- yl-2-{[4(4-tri- fluoromethyl-phenyl)pipe- rizin-1-yl]meth- yl}-5,6-dihydro-4H-imi- dazo[4,5,1-ij]quinoline |
| 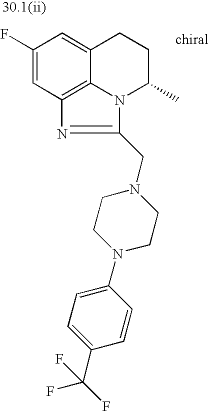 | [4S]-8-Fluoro-4-meth- yl-2-{[4(4-tri- fluoromethyl- phenyl)piperizin-1-yl]meth- yl}-5,6-di- hydro-4H-imi- dazo[4,5,1-ij]quinoline |

Preparation of Compounds

Compounds of the present invention can be prepared by various synthetic processes. The selection of a particular process to prepare a given compound is within the purview of the person of skill in the art. The choice of particular structural features and/or substituents may therefore influence the selection of one process over another.

Some starting materials for preparing compounds of the present invention are available from commercial chemical sources. Other starting compounds, as described below, are readily prepared from available precursors using straightforward transformations that are well known in the art.

Within these general guidelines, the compounds of formula I generally can be prepared according to the process illustrated in Scheme 1. Variables in Scheme 1 are as defined for formula I hereinabove unless otherwise specified.

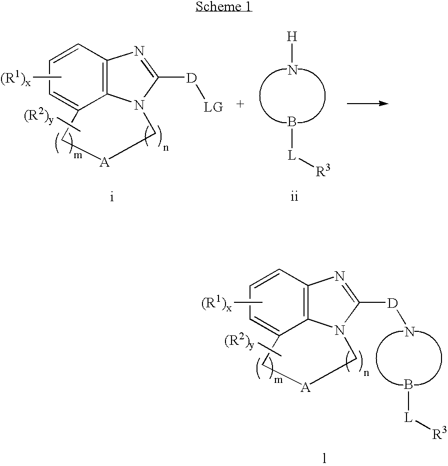

LG in Scheme 1 above represents a leaving group that is capable of being displaced by precursor (ii). Suitable leaving groups are well known in the art and thus include but are not limited to chloride, bromide, and sulphate esters such as mesylate and tosylate.

Precursor (i) can be prepared by a number of processes as described in more detail below. An exemplary process may be selected and/or adapted according to the particular structural features of a given precursor (i). Scheme 2 illustrates one exemplary process for making precursor (i). Thus, 6-Fluoro-2-methyl-1,2,3,4-tetrahydroquinoline was formylated using the mixed formic/acetic anhydride. This amide was then regioselectively nitrated with nitronium tetrafluoroborate. The formyl group was removed under basic hydrolytic conditions, and the nitro group was reduced to the aniline using hydrogen gas and palladium on carbon. The benzimidazole ring system was finally formed using chloroacetic acid or an equivalent in the presence of a mineral acid catalyst.

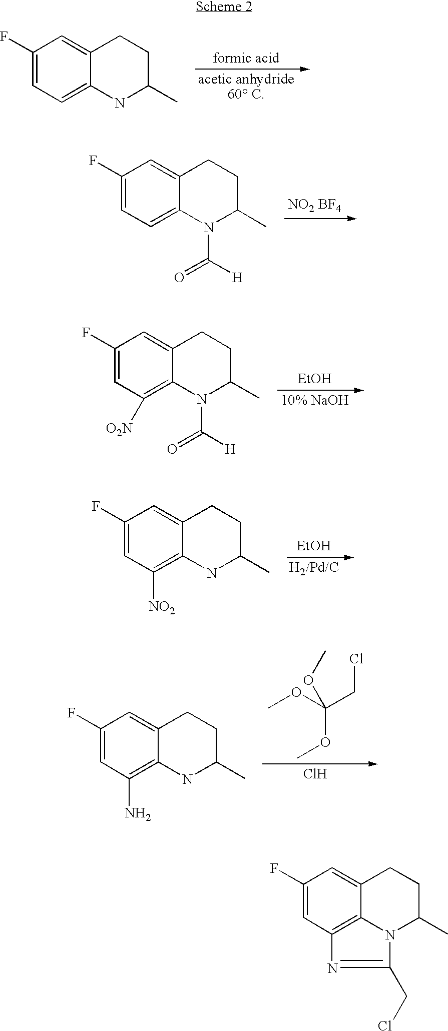

Scheme 3 illustrates another method for synthesizing precursor (i). 4-fluoroaniline was N-protected with a Boc-protecting group. This group was then used to direct ortho-lithiation, and subsequent trapping with 3-chloro-1-iodopropane provided 6-fluoro-3,4-dihydro-2H-quinoline-1-carboxylic acid tert-butyl ester. The Boc group was replaced with a formyl group, and the synthesis proceeded in an analogous fashion to Scheme 2.

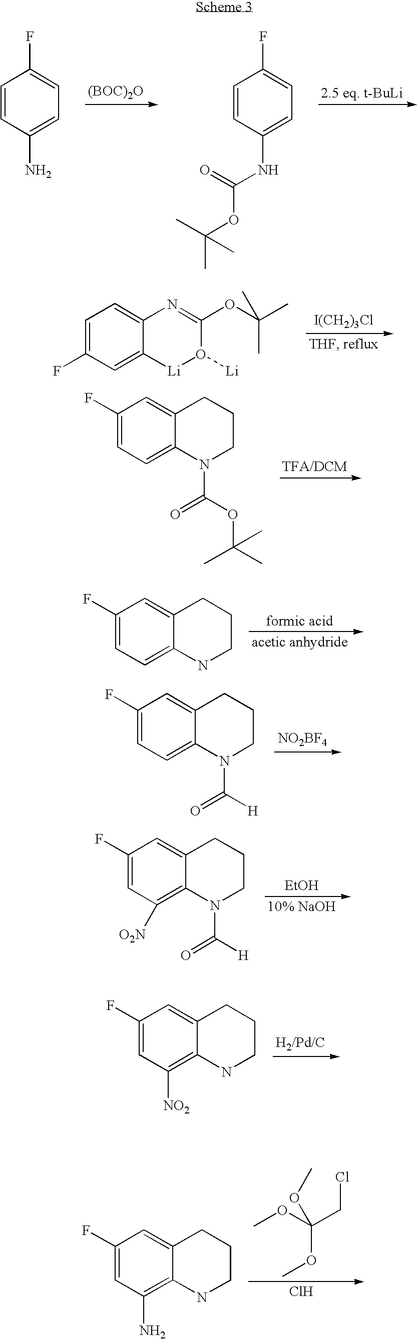

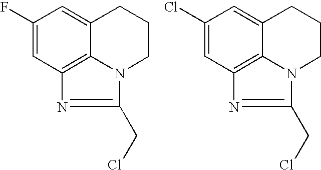

Scheme 4 illustrates another method for synthesizing precursor (i). Amino-3-nitrophenol was reacted with 1,2-dibromoethane under basic conditions to produce 5-nitro-3,4-dihydro-2H-1,4-benzoxazine. The synthesis then proceeded as outlined in Scheme 2.

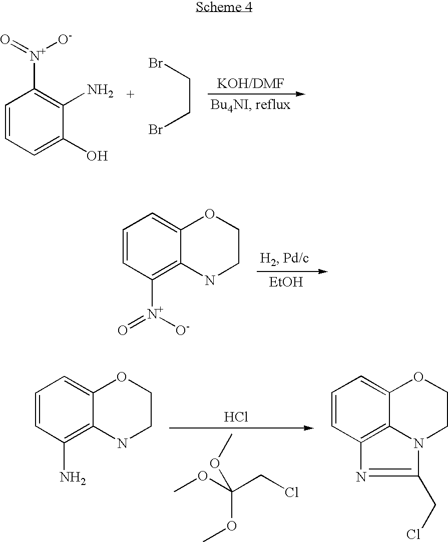

Scheme 5 illustrates another method for synthesizing precursor (i). 8-Nitroquinolone was reduced using hydrogen gas and platinum oxide catalyst. The resultant product was cyclized to the benzimidazole using chloroacetic acid or an equivalent in the presence of a mineral acid catalyst.

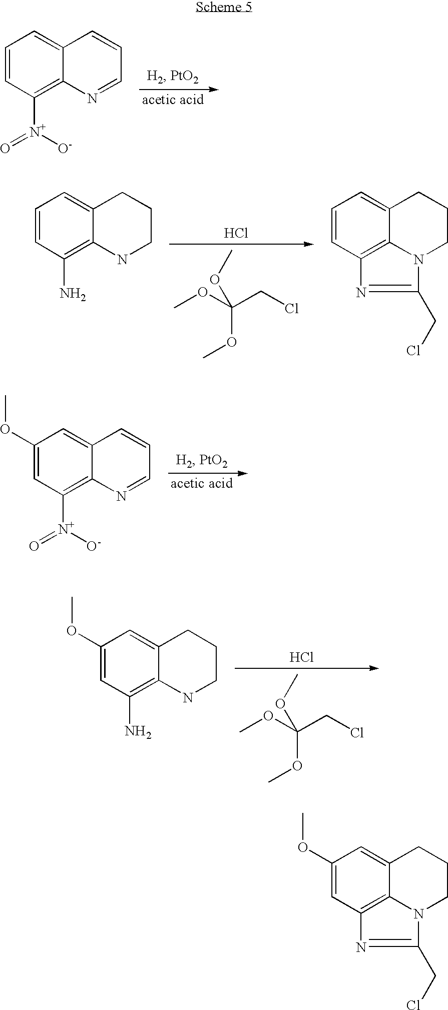

Scheme 6 illustrates another method for synthesizing precursor (i). This scheme is analogous to Scheme 2.

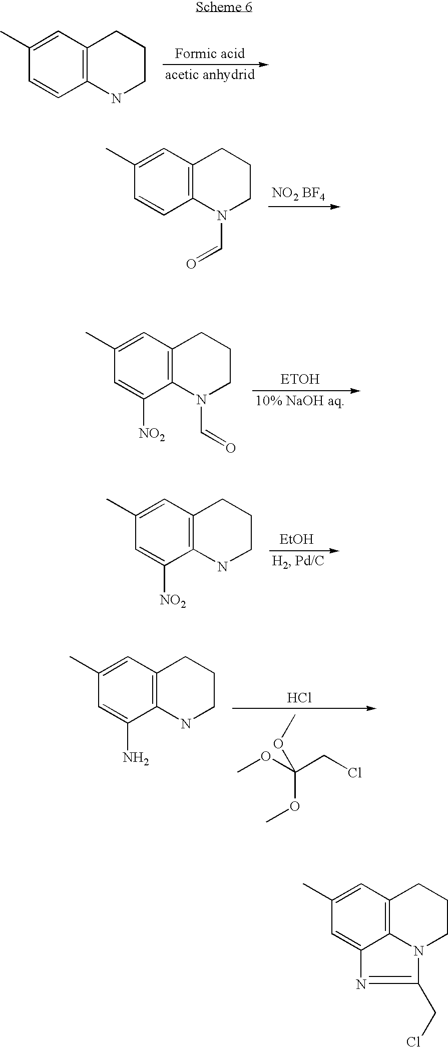

Precursor (ii) in Scheme 1 can be obtained from commercial sources or otherwise synthesized by using well known synthetic methodologies. In general, precursor (ii) can be prepared, if necessary, by the route depicted in Scheme 7 below. Boc-protected 4-piperidone was transformed to the vinyl triflate, which could in turn be converted into the cyclic boronate ester using standard conditions. This intermediate underwent Suzuki reaction conditions with various aryl halides in the presence of a palladium catalyst, yielding the final compounds after deprotection.

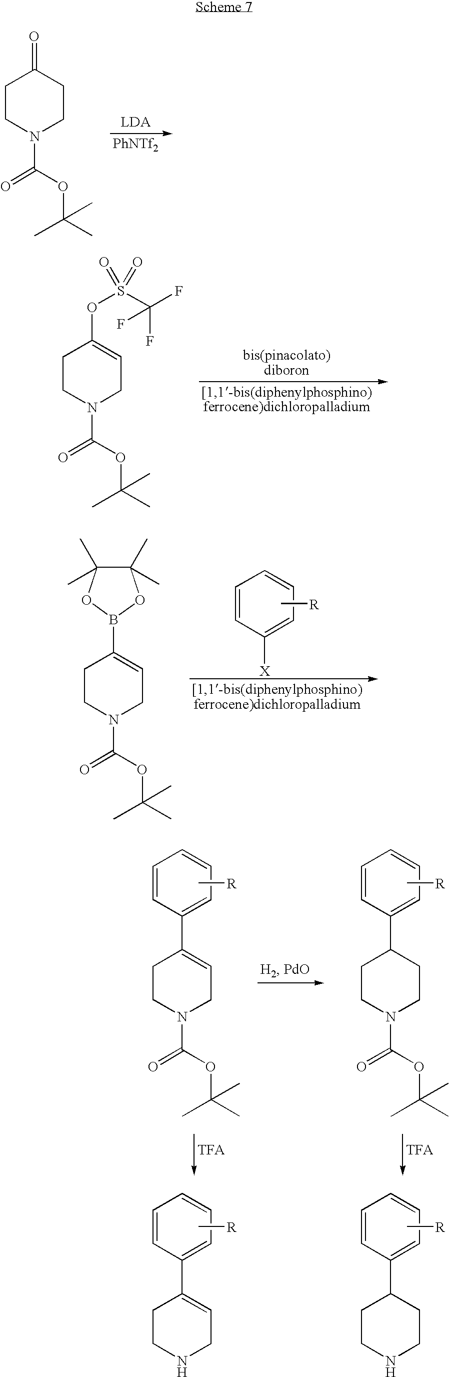

Pharmaceutical Composition

The compounds of the present invention may be formulated into conventional pharmaceutical composition comprising a compound of formula I, or a pharmaceutically acceptable salt or solvate thereof, in association with a pharmaceutically acceptable carrier or excipient. The pharmaceutically acceptable carriers can be either solid or liquid. Solid form preparations include, but are not limited to, powders, tablets, dispersible granules, capsules, cachets, and suppositories.

A solid carrier can be one or more substances, which may also act as diluents, flavoring agents, solubilizers, lubricants, suspending agents, binders, or table disintegrating agents. A solid carrier can also be an encapsulating material.

In powders, the carrier is a finely divided solid, which is in a mixture with the finely divided compound of the invention, or the active component. In tablets, the active component is mixed with the carrier having the necessary binding properties in suitable proportions and compacted in the shape and size desired.

For preparing suppository compositions, a low-melting wax such as a mixture of fatty acid glycerides and cocoa butter is first melted and the active ingredient is dispersed therein by, for example, stirring. The molten homogeneous mixture is then poured into convenient sized moulds and allowed to cool and solidify.

Suitable carriers include, but are not limited to, magnesium carbonate, magnesium stearate, talc, lactose, sugar, pectin, dextrin, starch, tragacanth, methyl cellulose, sodium carboxymethyl cellulose, low-melting wax, cocoa butter, and the like.

The term composition is also intended to include the formulation of the active component with encapsulating material as a carrier providing a capsule in which the active component (with or without other carriers) is surrounded by a carrier which is thus in association with it. Similarly, cachets are included.

Tablets, powders, cachets, and capsules can be used as solid dosage forms suitable for oral administration.

Liquid form compositions include solutions, suspensions, and emulsions. For example, sterile water or water propylene glycol solutions of the active compounds may be liquid preparations suitable for parenteral administration. Liquid compositions can also be formulated in solution in aqueous polyethylene glycol solution.

Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical formulation art. Exemplary compositions intended for oral use may contain one or more coloring, sweetening, flavoring and/or preservative agents.

Depending on the mode of administration, the pharmaceutical composition will include from about 0.05% w (percent by weight) to about 99% w, more particularly, from about 0.10% w to 50% w, of the compound of the invention, all percentages by weight being based on the total weight of the composition.

A therapeutically effective amount for the practice of the present invention can be determined by one of ordinary skill in the art using known criteria including the age, weight and response of the individual patient, and interpreted within the context of the disease which is being treated or which is being prevented.

Medical Use

We have discovered that the compounds of the present invention exhibit activity as pharmaceuticals, in particular as modulators of metabotropic glutamate receptors. More particularly, the compounds of the present invention exhibit activity as potentiators, more particularly as positive allosteric modulators, of the mGluR2 receptor, and are useful in therapy, in particular for the treatment of neurological and psychiatric disorders associated with glutamate dysfunction in an animal.

More specifically, the neurological and psychiatric disorders include, but are not limited to, disorders such as cerebral deficit subsequent to cardiac bypass surgery and grafting, stroke, cerebral ischemia, spinal cord trauma, head trauma, perinatal hypoxia, cardiac arrest, hypoglycemic neuronal damage, dementia (including AIDS-induced dementia), Alzheimer's disease, Huntington's Chorea, amyotrophic lateral sclerosis, ocular damage, retinopathy, cognitive disorders, idiopathic and drug-induced Parkinson's disease, muscular spasms and disorders associated with muscular spasticity including tremors, epilepsy, convulsions, cerebral deficits secondary to prolonged status epilepticus, migraine (including migraine headache), urinary incontinence, substance tolerance, substance withdrawal (including, substances such as opiates, nicotine, tobacco products, alcohol, benzodiazepines, cocaine, sedatives, hypnotics, etc.), psychosis, schizophrenia, anxiety (including generalized anxiety disorder, panic disorder, social phobia, obsessive compulsive disorder, and post-traumatic stress disorder (PTSD), mood disorders (including depression, mania, bipolar disorders), circadian rhythm disorders (including jet lag and shift work), trigeminal neuralgia, hearing loss, tinnitus, macular degeneration of the eye, emesis, brain edema, pain (including acute and chronic pain states, severe pain, intractable pain, neuropathic pain, inflammatory pain, and post-traumatic pain), tardive dyskinesia, sleep disorders (including narcolepsy), attention deficit/hyperactivity disorder, and conduct disorder.

The invention thus provides a use of any of the compounds according to formula I, or a pharmaceutically acceptable salt or solvate thereof, for the manufacture of a medicament for the treatment of any of the conditions discussed above.

Additionally, the invention provides a method for the treatment of a subject suffering from any of the conditions discussed above, whereby an effective amount of a compound according to formula I or a pharmaceutically acceptable salt or solvate thereof, is administered to a patient in need of such treatment. The invention also provides a compound of formula I or pharmaceutically acceptable salt or solvate thereof, as hereinbefore defined for use in therapy.

In the context of the present specification, the term “therapy” also includes “prophylaxis” unless there are specific indications to the contrary. The term “therapeutic” and “therapeutically” should be construed accordingly. The term “therapy” within the context of the present invention further encompasses the administration of an effective amount of a compound of the present invention, to mitigate either a pre-existing disease state, acute or chronic, or to mitigate a recurring condition. This definition also encompasses prophylactic therapies for prevention of recurring conditions and continued therapy for chronic disorders. In use for therapy in a warm-blooded animal such as a human, a compound of the present invention may be administered in the form of a conventional pharmaceutical composition by any route including orally, intramuscularly, subcutaneously, topically, intranasally, intraperitoneally, intrathoracically, intravenously, epidurally, intrathecally, intracerebroventricularly and by injection into the joints. In preferred embodiments of the invention, the route of administration is oral, intravenous, or intramuscular.

The dosage will depend on the route of administration, the severity of the disease, age and weight of the patient and other factors normally considered by the attending physician, who determines the individual regimen and dosage level for a particular patient.

As mentioned above, the compounds described herein may be provided or delivered in a form suitable for oral use, for example, in a tablet, lozenge, hard and soft capsule, aqueous solution, oily solution, emulsion, and suspension. Alternatively, the compounds may be formulated into a topical administration, for example, as a cream, ointment, gel, spray, or aqueous solution, oily solution, emulsion or suspension. The compounds described herein also may be provided in a form that is suitable for nasal administration, for example, as a nasal spray, nasal drops, or dry powder. The compounds can be administered to the vagina or rectum in the form of a suppository. The compounds described herein also may be administered parentally, for example, by intravenous, intravesicular, subcutaneous, or intramuscular injection or infusion. The compounds can be administered by insufflation (for example as a finely divided powder). The compounds may also be administered transdermally or sublingually.

In addition to their use in therapeutic medicine, the compounds of formula I, or salts thereof, are useful as pharmacological tools in the development and standardization of in vitro and in vivo test systems for the evaluation of the effects of inhibitors of mGluR-related activity in laboratory animals as part of the search for new therapeutics agents. Such animals include, for example, cats, dogs, rabbits, monkeys, rats and mice.

The invention is further illustrated by way of the following examples, which are intended to elaborate several embodiments of the invention. These examples are not intended to, nor are they to be construed to, limit the scope of the invention. It will be clear that the invention may be practiced otherwise than as particularly described herein. Numerous modifications and variations of the present invention are possible in view of the teachings herein and, therefore, are within the scope of the invention.

General Methods

All starting materials are commercially available or earlier described in the literature. The ¹H and ¹³C NMR spectra were recorded either on Bruker 300, Bruker DPX400 or Varian +400 spectrometers operating at 300, 400 and 400 MHz for ¹H NMR respectively, using TMS or the residual solvent signal as reference, in deuterated chloroform as solvent unless otherwise indicated. All reported chemical shifts are in ppm on the delta-scale, and the fine splitting of the signals as appearing in the recordings (s: singlet, br s: broad singlet, d: doublet, t: triplet, q: quartet, m: multiplet).

Analytical in line liquid chromatography separations followed by mass spectra detections, were recorded on a Waters LCMS consisting of an Alliance 2795 (LC) and a ZQ single quadropole mass spectrometer. The mass spectrometer was equipped with an electrospray ion source operated in a positive and/or negative ion mode. The ion spray voltage was ±3 kV and the mass spectrometer was scanned from m/z 100-700 at a scan time of 0.8 s. To the column, X-Terra MS, Waters, C8, 2.1×50 mm, 3.5 mm, was applied a linear gradient from 5% to 100% acetonitrile in 10 mM ammonium acetate (aq.), or in 0.1% TFA (aq.).

Preparative reversed phase chromatography was run on a Gilson autopreparative HPLC with a diode array detector using an XTerra MS C8, 19×300 mm, 7 mm as column.

Purification by a chromatotron was performed on rotating silica gel/gypsum (Merck, 60 PF-254 with calcium sulphate) coated glass sheets, with coating layer of 1, 2, or 4 mm using a TC Research 7924T chromatotron.

Purification of products were also done using Chem Elut Extraction Columns (Varian, cat #1219-8002), Mega BE-SI (Bond Elut Silica) SPE Columns (Varian, cat #12256018; 12256026; 12256034), or by flash chromatography in silica-filled glass columns. Microwave heating was performed in a Smith Synthesizer Single-mode microwave cavity producing continuous irradiation at 2450 MHz (Personal Chemistry AB, Uppsala, Sweden).

The pharmacological properties of the compounds of the invention can be analyzed using standard assays for functional activity. Examples of glutamate receptor assays are well known in the art as described in, for example, Aramori et al., 1992, Neuron, 8:757; Tanabe et al., 1992, Neuron, 8:169; Miller et al., 1995, J. Neuroscience, 15:6103; Balazs, et al., 1997, J. Neurochemistry, 1997,69:151. The methodology described in these publications is incorporated herein by reference. Conveniently, the compounds of the invention can be studied by means of an assay that measures the mobilization of intracellular calcium, [Ca²⁺]ᵢ in cells expressing mGluR2.

Fluorometric Imaging Plate Reader (FLIPR) analysis was used to detect allosteric activators of mGluR2 via calcium mobilization. A clonal HEK 293 cell line expressing a chimeric mGluR2/CaR construct comprising the extracellular and transmembrane domains of human mGluR2 and the intracellular domain of the human calcium receptor, fused to the promiscuous chimeric protein G_{aqi5} was used. Activation of this construct by agonists or allosteric activators resulted in stimulation of the PLC pathway and the subsequent mobilization of intracellular Ca²⁺ which was measured via FLIPR analysis. At 24-hours prior to analysis, the cells were trypsinized and plated in DMEM at 100,000 cells/well in black sided, clear-bottom, collagen I coated, 96-well plates. The plates were incubated under 5% CO₂ at 37° C. overnight. Cells were loaded with 6 μM fluo-3 acetoxymethylester (Molecular Probes, Eugene Oreg.) for 60 min. at room temperature. All assays were performed in a buffer containing 126 mM NaCl, 5 mM KCl, 1 mM MgCl₂, 1 mM CaCl₂, 20 mM Hepes, 0.06 μM DCG-IV (a Group II mGluR selective agonist), supplemented with 1.0 mg/ml D-glucose and 1.0 mg/ml BSA fraction IV (pH 7.4).

FLIPR experiments were done using a laser setting of 0.8 W and a 0.4 second CCD camera shutter speed. Extracellular fluo-3 was washed off and cells were maintained in 160 μL of buffer and placed in the FLIPR. An addition of test compound (0.01 μM to 30 μM in duplicate) was made after 10 seconds of baseline fluorescent readings were recorded on FLIPR. Fluorescent signals were then recorded for an additional 75 seconds at which point a second addition of DCG-IV (0.2 μM) was made and fluorescent signals were recorded for an additional 65 seconds. Fluorescent signals were measured as the peak height of the response within the sample period. Data was analyzed using Assay Explorer, and EC₅₀ and Eₘₐₓ values (relative to maximum DCG-IV effect) were calculated using a four parameter logistic equation.

A [³⁵S]-GTPγS binding assay was used to functionally assay mGluR2 receptor activation. The allosteric activator activity of compounds at the human mGluR2 receptor was measured using a [³⁵S]-GTPγS binding assay with membranes prepared from CHO cells which stably express the human mGluR2. The assay is based upon the principle that agonists bind to G-protein coupled receptors to stimulate GDP-GTP exchange at the G-protein. Since [³⁵S]-GTPγS is a non-hydrolyzable GTP analog, it can be used to provide an index of GDP-GTP exchange and, thus, receptor activation. The GTPγS binding assay therefore provides a quantitative measure of receptor activation.

Membranes were prepared from CHO cells stably transfected with human mGluR2. Membranes (30 μg protein) were incubated with test compound (3 nM to 300 μM) for 15 min. at room temperature prior to the addition of 1 μM glutamate, and incubated for 30 min at 30° C. in 500 μl assay buffer (20 mM HEPES, 100 mM NaCl, 10 mM MgCl₂), containing 30 μM GDP and 0.1 nM [³⁵S]-GTPγS (1250 Ci/mmol). Reactions were carried out in triplicate in 2 ml polypropylene 96-well plates. Reactions were terminated by vacuum filtration using a Packard 96-well harvester and Unifilter-96, GF/B filter microplates. The filter plates were washed 4×1.5 ml with ice-cold wash buffer (10 mM sodium phosphate buffer, pH 7.4). The filter plates were dried and 35 μl of scintillation fluid (Microscint 20) was added to each well. The amount of radioactivity bound was determined by counting plates on the Packard TopCount. Data was analyzed using GraphPad Prism, and EC₅₀ and Eₘₐₓ values (relative to the maximum glutamate effect) were calculated using non-linear regression.

The following abbreviations are used in the examples:

BOC tert-butoxycarbonyl

BSA Bovine Serum Albumin

CCD Charge Coupled Device

CRC Concentration Response Curve

DBU 1,8-diazabicyclo[5.4.0]undec-7-ene

DCM dichloromethane

DHPG 3,5-dihydroxyphenylglycine;

DIBAL diisobutylaluminum hydride

DMF N,N-dimethylformamide

DMSO dimethyl sulfoxide

EDTA Ethylene Diamine Tetraacetic Acid

Et₃N triethylamine

EtOAc Ethyl acetate

FLIPR Fluorometric Imaging Plate reader

GC/MS gas chromatograph coupled mass spectroscopy

GHEK Human Embryonic Kidney expressing Glutamate Transporter

HEPES 4-(2-hydroxyethyl)-1-piperazineethanesulfonic acid (buffer)

IP₃ inositol triphosphate

MCPBA 3-chloroperbenzoic acid

MeOH methanol

NMP N-Methylpyrrolidinone

NMR nuclear magnetic resonance

PCC pyridinium chlorochromate

ppm parts per million

RT room temperature

SPE solid phase extraction

TFA trifluoroacetic acid

THF tetrahydrofuran

TLC thin layer chromatography

Generally, the compounds of the present invention were active in the assays described herein at concentrations (or with EC₅₀ values) of less than 10 μM. Preferred compounds of the invention have EC₅₀ values of less than 1 μM; more preferred compounds of less than about 100 nM. For example, the compounds of Examples 26.55, 26.56, 26.65, 26.69, and 28.1 have EC₅₀ values of 0.37, 1.58, 0.08, 0.23, and 1.11 μM, respectively.

Preparation of Intermediate Compounds

Preparation of Precursor (i)

### EXAMPLE 1.1

### Tert-butyl(4-fluorophenyl)carbamate

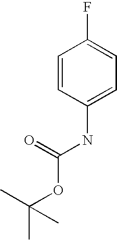

To a solution of (4-fluorophenyl)-amine (5 g, 45 mmol) in THF (200 ml), di-tert-butyl dicarbonate (10.8 g, 50 mmol) was added. The resulting mixture was fluxed for 3 h. After removal of solvents, the residue was dissolved in EtOAc and washed with 10% citric acid, water, brine, and dried over anhydrous sodium sulphate, filtered and concentrated to get a brown solid. This brown solid was washed with hexanes to provide a white solid (8.5 g, 89%). ¹H NMR (300 MHz, CDCl₃): δ 7.28-7.40 (m, 2H), 6.97-7.02 (m, 2H), 6.50 (s, 1H), 1.53 (s, 9H).

In a similar fashion, the following compounds were made:

### EXAMPLE 2.1

### Tert-butyl-6-fluoro-3,4-dihydroquinoline-1(2H)-carboxylate

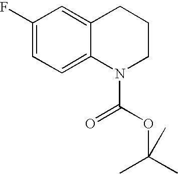

A 1.6M solution of tert-butyllithium in pentane (37 ml, 59.17 mmol) was added in a dropwise fashion to a solution of tert-butyl(4-fluorophenyl)carbamate (5 g, 23.67 mmol) in anhydrous THF (200 ml, Argon atmosphere) at −78° C. After 15 min at −78° C., the reaction was allowed to warm to −20° C. where it was maintained for 3 h. The resulting solution of ortho lithiated compound was then quenched at −20° C. with the solution of 1-chloro-3iodopropane (2.8 ml, 26.03 mmol) in anhydrous THF (20 ml), stirred a further 20 min, and then refluxed overnight. The reaction mixture was then quenched with water and the product was extracted with DCM. The organic layer was washed with brine and dried over anhydrous Na₂SO₄, filtered, and concentrated in vacuo. Purification by flash chromatography (silica gel, gradient elution with 5% to 10% ethyl acetate/hexanes) provided a yellow oil (2.85 g, 48%). ¹H NMR (300 MHz, CDCl₃): δ 7.55-7.59 (m, 1H), 6.69-6.78(m, 2H), 3.62-3.66 (m, 2H), 2.68 (t, 2H), 1.83-1.87 (m, 2H), 1.48 (s, 9H).

In a similar fashion, the following compound was made:

| | | | |
|---|---|---|---|
| Ex- am- ple 2.2 | 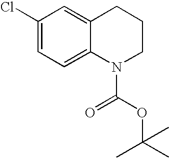 | Tert-butyl-6-chloro-3,4-di- hydroquino- line-1(2H)-carboxy- late | 5.4 g (77%), yellow solid |

| | |
|---|---|
| NMR | 7.61-7.64(m, 1H), 7.03-7.10(m, 2H), 3.67-3.71(m, 2H), |
| | 2.71(t, 2H), 1.87-1.91(m, 2H), 1.51(s, 9H). |

### EXAMPLE 3.1

### 6-fluoro-2-methyl-3,4-dihydroquinoline-1(2H)-carbaldehyde

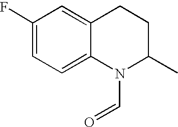

The mixture of formic acid (28 ml, 726.3 mmol) and acetic anhydride (23 ml, 242 mmol) was added to 6-fluoro-2-methyl-1,2,3,4-tetrahydroquinoline (4 g, 24.2 mmol) dropwise. The resulting mixture was stirred at 60° C. for 1 h. After removal of solvents, the residue was basified with 3N aqueous sodium hydroxide solution. The product with extracted with dichloromethane and the organic layer was washed with brine and dried over anhydrous Na₂—SO₄. The solvent was removed under reduced pressure to afford a red oil (4.8 g, yield 103%). ¹H NMR (300 MHz, CDCl₃): δ 8.53 (s, 1H), 6.99-7.04 (m, 1H), 6.82-6.87 (m, 2H), 4.73 (t, 1H), 2.50-2.70 (m, 2H), 2.02-2.10 (m, 1H), 1.58-1.64 (m, 1H), 1.12 (d, 3H).

In a similar fashion, the following compound was made:

| | | | |
|---|---|---|---|
| Ex- ample 3.2 | 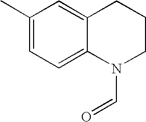 | 6-methyl-3,4-di- hydroquino- line-1(2H)-carb- aldehyde | 2.2 g (94%), red oil |

| | |
|---|---|
| NMR | 8.73(s, 1H), 6.96-7.03(m, 3H), 3.75-3.79(m, 2H), |
| | 2.75(t, 2H), 2.29(s, 3H), 1.81-1.89(m, 2H) |

### EXAMPLE 4.1

### 6-fluoro-3,4-dihydroquinoline-1(2H)-carbaldehyde

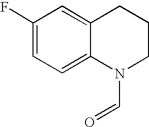

Tert-butyl-6-fluoro-3,4-dihydroquinoline-1(2H)-carboxylate (2.8 g, 11.16 mmol) was dissolved in the mixture of TFA and DCM (10 ml, v/v=1:1) and stirred overnight. After removal of solvents, the mixture of formic acid (10.5 ml, 278.9 mmol) and acetic anhydride (8.4 ml, 89.3 mmol) was added to the residue dropwise. The resulting mixture was stirred at 60° C. for 1 h. After removal of solvents, the residue was basified with 3N aqueous sodium hydroxide solution. The product with extracted with dichloromethane and the organic layer was washed with brine, dried over anhydrous Na₂SO₄. The solvent was removed under reduced pressure to afford a red oil (1.4 g, yield 70%). ¹H NMR (300 MHz, CDCl₃): δ 8.61(s, 1H), 6.99-7.04 (m, 1H), 6.72-6.84 (m, 2H), 3.68-3.72 (m, 2H), 2.71 (t, 2H), 1.81-1.89 (m, 2H).

In a similar fashion, the following compounds were made:

| | | | |
|---|---|---|---|
| Ex- ample 4.2 | 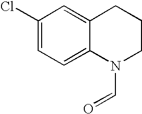 | 6-chloro-1,2,3,4-tetra- hydroquino- line-8-amine | 3.35 g (85%), off-white solid |

| | |
|---|---|
| NMR | 8.77(s, 1H), 7.05-7.20(m, 2H), 7.08-7.11(m, 1H) |

### EXAMPLE 5.1

### 6-fluoro-2-methyl-8-nitro-3,4-dihydroquinoline-1(2H)-carbaldehyde

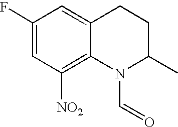

To a suspension of nitronium tetrafluoroborate (8.2 g, 58.7 mmol) in dichloromethane (50 ml), the solution of 6-fluoro-2-methyl-3,4-dihydroquinoline-1(2H)-carbaldehyde(4.8g, 24.84 mmol) in dichloromethane (20 ml) was added dropwise at 0° C. The reaction was stirred at 0° C. for 2 h., then poured into ice-water (40 ml), the product was extracted with dichloromethane. The combined organic layers were washed with water, brine, and dried over anhydrous Na₂SO₄. The solvent was removed in vacuo to afford a yellow solid (5.8 g, 97%), confirmed by GC/MS.

In a similar fashion, the following compounds were made:

| | | | |
|---|---|---|---|
| Ex- ample 5.2 | 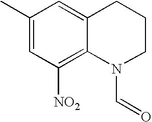 | 6-meth- yl-8-nitro-3,4-di- hydro- quinoline-1(2H)-carb- aldehyde | 1.2 g (97%), yellow solid |
| Ex- ample 5.3 | 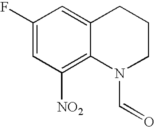 | 6-fluoro-8-nitro-3,4-di- hydro- quinoline-1(2H)-carb- aldehyde | 1.6 g (91%), yellow solid |
| Ex- ample 5.4 | 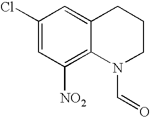 | 6-chloro-8-nit- ro-3,4-di- hydro- quinoline-1(2H)-carb- aldehyde | 3.9 g (95%), yellow solid |

### EXAMPLE 6.1

### 6-fluoro-2-methyl-8-nitro-1,2,3,4-tetrahydroquinoline

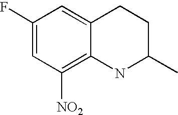

A suspension of 6-fluoro-2-methyl-8-nitro-3,4-dihydroquinoline-1(2H)-carbaldehyde (5.8 g, 24.3 mmol) in the mixture of ethanol and 10% NaOH aqueous solution (100 ml, v/v=1:1) was refluxed over night. After cooled to room temperature, the reaction mixture was diluted with water (200 ml), the product was extracted with dichloromethane. The organic layer was washed with brine, dried over anhydrous Na₂SO₄. The solvent was removed in vacuo to afford a red solid (5.0 g, 98%), confirmed by GC/MS.

In a similar fashion, the following compounds were made:

| Ex- am- ple 6.2 | 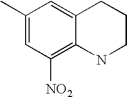 | 6-meth- yl-8-nitro-1,2,3,4-tet- rahydroquinoline | 340 mg (78%), red solid |
|---|---|---|---|
| NMR | 8.26(s, 1H), 7.76(m, 1H), 6.97(s, 1H), 3.49-3.54(m, 2H), | | |
| | 2.80(t, 2H), 2.20(s, 3H), 1.91-1.99(m, 2H) | | |
| Ex- am- ple 6.3 | 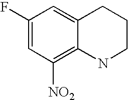 | 6-fluoro-8-nitro-1,2,3,4-tet- rahydroquinoline | 1.2 g (100%), red solid |
| NMR | 8.27(s, 1H), 7.58-7.62(m, 1H), 6.92-6.95(m, 1H), | | |
| | 3.49-3.54(m, 2H), 2.82(t, 2H), 1.91-1.99(m, 2H) | | |
| Ex- am- ple 6.4 | 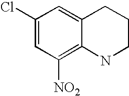 | 6-fluoro-8-nitro-1,2,3,4-tet- rahydroquinoline | 3.1 g (90%), orange solid |

| | |
|---|---|
| NMR | 8.36(s, 1H), 7.99(d, 1H), 7.10-7.11(m, 1H), |
| | 3.53-3.57(m, 2H), 2.84(t, 2H), 2.20(s, 3H), 1.94-2.02(m, 2H) |

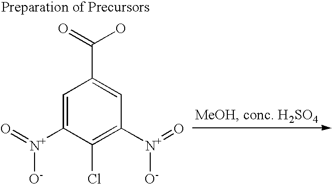

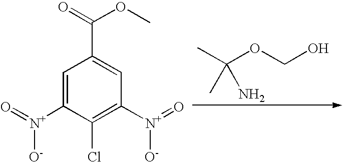

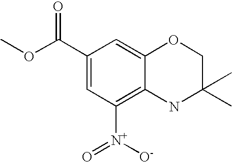

### EXAMPLE 7.1

### 4-Chloro-3,5-dinitro-benzoic acid methyl ester

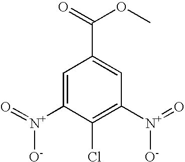

To a solution of 4-chloro-3,5-dinitro-benzoic acid (2 g, 7.8 mmol) in methanol (10 mL) was added concentrated H₂SO₄ (1 mL) dropwise. The reaction mixture was refluxed for 5 h. The reaction mixture was then cooled to room temperature and kept in 0° C. bath for 30 min. Product was as obtained off-white solid after filtration (2.10 g, quantitative yield). ¹H NMR (300 MHz, CDCl₃): δ 8.62 (s, 2H), 4.05 (s, 3H).

### EXAMPLE 8.1

### 3,3-Dimethyl-5-nitro-3,4-dihydro-2H-benzo[1,4]oxazine-7-carboxylic acid methyl ester

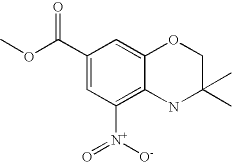

To a solution of 2-amino-2-methyl-propan-1-ol (1.44 g, 16.16 mmol) in methanol (15 mL) was added 4-Chloro-3,5-dinitro-benzoic acid methyl ester (2.1 g, 8.08 mmol). The resulting reaction mixture was refluxed for 45 min. After the reaction mixture was cooled to room temperature, sodium methoxide (1.22 g, 22.58 mmol) was added slowly, and then the reaction mixture was refluxed for 45 min. The reaction mixture was cooled to room temperature and ice-water was added. The precipitate obtained by filtration was purified on silica gel eluting with 10˜20% ethyl acetate in hexane to give the product as yellow solid (900 mg, 42%). ¹H NMR (300 MHz, CDCl₃): δ 8.54 (d, 1H), 8.12 (br, 1H), 7.64 (d, 1H), 3.91 (s, 5H), 1.4 (s, 6H).

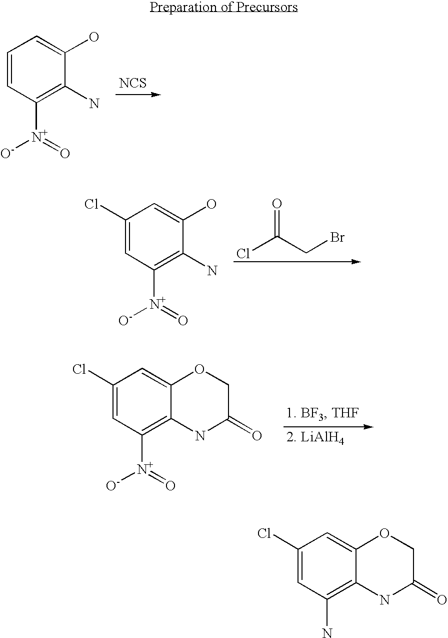

### EXAMPLE 9.1

### 2-Amino-5-chloro-3-nitro-phenol

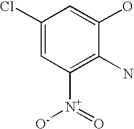

The solution of 2-amino-3-nitro-phenol (3 g, 19.46 mmol) and N-chlorosuccinimide (3.12 g, 23.35 mmol) in acetonitrile (100 mL) was refluxed for 3 h. The reaction mixture was concentrated and the residue was dissolved in ethyl acetate. The mixture was washed with water and brine. The organic phase was dried over anhydrous sodium sulfate and concentrated in vacuo to give product as red solid (3.7 g, quantitative yield). ¹H NMR (300 MHz, CDCl₃): δ 7.55 (d, 1H), 6.83 (d, 1H).

### EXAMPLE 10.1

### 7-Chloro-5-nitro-4H-benzo[1,4]oxazin-3-one

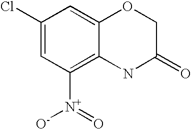

2-Amino-5-chloro-3-nitro-phenol (3.7 g, 19.46 mmol) was dissolved in acetonitrile (100 mL). Bromo-acetyl chloride (3.37 g, 21.40 mmol) was added followed by potassium carbonate (6.72 g, 48.65 mmol). The reaction mixture was refluxed overnight. After removal of the solvent, the residue was partitioned between ethyl acetate and water. The aqueous phase was extracted with ethyl acetate (2×). The combined organic phases were washed with brine, dried over anhydrous sodium sulfate and concentrated in vacuo. The crude residue was purified on silica gel eluting with 20˜50% ethyl acetate in hexane to give product as brown solid (2.4 g, 54%). ¹H NMR (300 MHz, CDCl₃): δ 7.94 (d, 1H), 7.31 (d, 1H), 4.74 (s, 2H).

### EXAMPLE 11.1

### 5-Amino-7-chloro-4H-benzo[1,4]oxazin-3-one

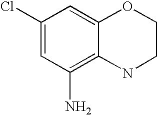

Lithium aluminum hydride (1.07 g, 22.4 mmol) was added to the suspension of 7-chloro-5-nitro-4H-benzo[1,4]oxazin-3-one (1.2 g, 5.3 mmol) in THF (30 mL). The reaction mixture was stirred at room temperature for overnight. The reaction mixture was then quenched with water, the aqueous phase was extracted with ethyl acetate; combined organic phases were washed with water and brine, dried over anhydrous sodium sulfate and concentrated in vacuo. The crude residue was purified on silica gel eluting with 60% ethyl acetate in hexane and 2% methanol in ethyl acetate to give the product as red oil (460 mg, 47%). ¹H NMR (300 MHz, CDCl₃): δ 6.39 (d, 1H), 6.33 (d, 1H), 4.17 (m, 2H), 3.53 (br, 2H), 3.41 (m, 2H), 3 (br, 1H).

### EXAMPLE 12.1

### 6-fluoro-2-methyl-1,2,3,4-tetrahydroquinoline-8-amine

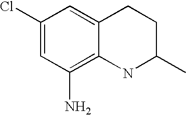

To a solution of 6-fluoro-2-methyl-8-nitro-1,2,3,4-tetrahydroquinoline (4.5 g, 21.43 mmol) in ethanol (100 ml), 10% Pd/carbon (600 mg) was added, followed by charging a hydrogen-filled balloon on the top of flask. The reaction was stirred at room temperature for 36 h. The reaction mixture was filtered through diatomaceous earth and filtrate was condensed to get a colorless oil (3.7 g, 96%), confirmed by GC/MS.

In a similar fashion, the following compounds were made:

| Ex- am- ple 12.2 | 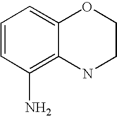 | 3,4-di- hydro-2H-1,4-benz- oxazin-5-amine | 390 mg (100%), colorless oil |
|---|---|---|---|
| Ex- am- ple 12.3 | 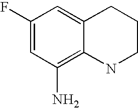 | 6-fluoro-1,2,3,4-tetra- hydroquinolin-8-amine | 1.20 g (100%), red solid |
| NMR | 8.27(s, 1H), 7.60(dxd, 1H), 6.92-6.95(m, 1H), 3.49-3.54(m, 2H), | | |
| | 2.82(t, 2H), 1.91-1.99(m, 2H) | | |
| Ex- am- ple 12.4 | 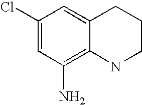 | 6-chloro-1,2,3,4-tetra- hydroquinoline-8-amine | 3.1 g, (90%), yellow solid |
| NMR | 8.36(s, 1H), 7.8(d, 1H), 7.10-7.11(m, 1H), 3.53-3.57(m, 2H), | | |
| | 2.84(t, 2H), 1.94-2.02(m, 2H) | | |
| Ex- am- ple 12.5 | 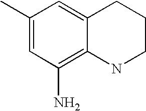 | 6-methyl-1,2,3,4-tetra- hydroquinoline-8-amine | 360 mg, (92%), yellow oil |

### EXAMPLE 13.1

### 1,2,3,4-tetrahydroquinoline-8-amine

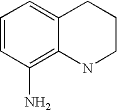

A suspension of 8-nitroquinoline (500 mg, 2.87 mmol) and PtO₂(16 mg, 0.072 mmol) in glacial acetic acid (5 ml) was stirred with H₂-balloon on the top for three days. After removal of acetic acid, the residue was partitioned between dichloromethane and saturated sodium bicarbonate solution. The aqueous layer was back-extracted with DCM, and combined organic layers were washed with brine, dried over anhydrous Na₂SO₄, filtered, and concentrated in vacuo. Purification by flash chromatography (silica gel, elution with 10% ethyl acetate/DCM) provided a red oil (120 mg, 24%), confirmed by GC/MS.

In a similar fashion, the following compounds were made:

| | | | |
|---|---|---|---|
| Example 13.2 | 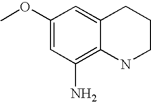 | 6-meth- oxy-1,2,3,4-tetra- hydro- quinoline-8-amine | 30 mg (5.6%), red oil |

### EXAMPLE 14.1

### 2-(chloromethyl)-8-fluoro-4-methyl-5,6-dihydro-4H-imidazo[4,5,1-ij]quinoline

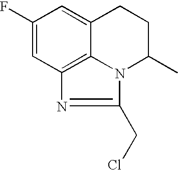

To a suspension of 6-fluoro-2-methyl-1,2,3,4-tetrahydroquinoline-8-amine (3.7 g, 20.6 mmol) in 2-chloro-1,1,1-trimethoxyethane (25 ml), conc. HCl (3 ml) was added. The resulting solution was stirred over night. The reaction mixture was diluted with dichloromethane and basified with saturated sodium bicarbonate solution. The aqueous layer was back-extracted with dichloromethane, and the combined organic layers were washed with brine, dried over anhydrous Na₂SO₄, filtered, and concentrated in vacuo. Purification by flash chromatography (silica gel, gradient elution with 60% ethyl acetate/hexanes to 5% 2M NH₃ in methanol/(60% ethyl acetate/hexanes)) provided a yellow solid (2.8 g, 56%). ¹H NMR (300 MHz, CDCl₃): δ 7.17(dxd, 1H), 6.78(d, 1H), 4.73-4.78 (m, 3H), 2.81-2.99 (m, 2H), 2.07-2.13 (m, 2H), 1.42 (d, 3H).

In a similar fashion, the following compounds were made:

| Example 14.2 | 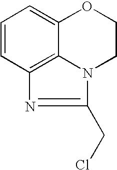 | 2-(chloromethyl)-4,5-di- hydroimidazo[1,4,5-de][1,4]benz- oxazine | 430 mg (84%), brown solid |
|---|---|---|---|
| NMR | 7.27(dxd, 1H), 7.09(t, 1H), 6.69-6.72(m, 1H), 4.71(s, 2H), 4.38-4.72(m, 2H), 4.25-4.29(m, | | |
| | 2H) | | |
| Example 14.3 | 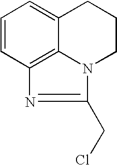 | 2-(chloromethyl)-5,6-di- hydro-4H-imi- dazo[4,5,1-ij]quinoline | 140 mg (94%) brown solid |
| NMR | 7.56(dxd, 1H), 7.20(t, 1H), 7.03-7.06(m, 1H), 4.82(s, 2H), 4.22-4.26(m, 2H), 2.99(t, | | |
| | 2H), 2.25-2.29(m, 2H) | | |
| Example 14.4 | 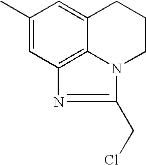 | 2-(chloromethyl)-8-meth- yl-5,6-dihydro-4H-imi- dazo[4,5,1-ij]quinoline | 360 mg (92%) brown solid |
| NMR | 7.31(d, 1H), 6.86(s, 1H), 4.75(s, 2H), 4.13-4.17(m, 2H), 2.89(t, 2H), 2.45(s, 3H), | | |
| | 2.18-2.21(m, 2H) | | |
| Example 14.5 | 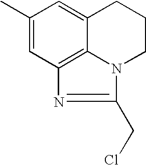 | 2-(chloromethyl)-8-meth- oxy-5,6-dihydro-4H-imi- dazo[4,5,1-ij]quinoline | 17 mg (43%), brown oil |
| NMR | 7.03(d, 1H), 6.74-6.75(m, 1H), 4.83(s, 2H), 4.23-4.27(m, 2H), 3.85(s, 3H), 2.96(t, | | |
| | 2H), 2.26-2.30(m, 2H) | | |
| Example 14.6 | 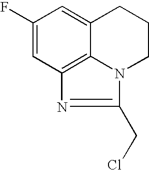 | 2-(chloromethyl)-8-fluoro-5,6-di- hydro-4H-imi- dazo[4,5,1-ij]quinoline | 1 g (62%), yellow solid |
| NMR | 7.12-7.16(m, 1H), 6.72-6.76(m, 1H), 4.73(s, 2H), 4.14-4.17(m, 2H), 2.85-2.90(t, | | |
| | 2H), 2.17-2.21(m, 2H) | | |
| Example 14.7 | 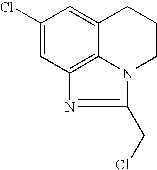 | 8-chloro-2-(chloro- methyl)-5,6-di- hydro-4H-imi- dazo[4,5,1-ij]quinoline | 1.7 g (62%), off- white solid |
| NMR | 7.54-7.55(m, 1H), 7.04-7.05(m, 1H), 4.82(s, 2H), 4.24-4.28(m, 2H), 2.95-2.99(m, | | |
| | 2H), 2.26-2.32(m, 2H) | | |
| Example 14.8 | 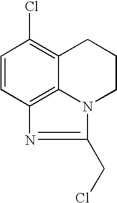 | 7-Chloro-2-chloromethyl-5,6-di- hydro-4H-imi- dazo[4,5,1-ij]quino- line | 110 mg, brown solid |
| NMR | 7.5(d, 1H), 7.21(d, 1H), 4.83(s, 2H), 2.55(t, 2H), 3.01(t, 2H), 2.31(m, 2H) | | |
| Example 14.9 | 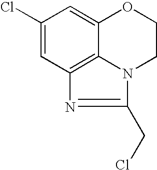 | 7-Chloro-2-chloromethyl-3,4-di- hydro-5-oxa-1,2a-di- aza-acenaphthylene | 400 mg, brown solid, 100% |
| NMR | 7.34(s, 1H), 6.81(s, 1H), 4.84(s, 2H), 4.54(m, 2H), 4.43(m, 2H) | | |
| Example 14.10 | 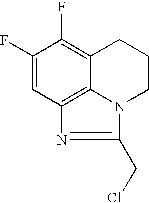 | 2-Chloromethyl-7,8-di- fluoro-5,6-di- hydro-4H-imidazo[4,5,1-ij]quino- line | 10 mg, brown oil, 3.6% |
| NMR | 7.36(m, 1H), 4.82(s, 2H), 4.25(t, 2H), 3.05(m, 2H), 2.31(m, 2H) | | |
| Example 14.11 | 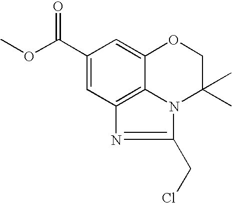 | 2-Chloromethyl-3,3-di- methyl-3,4-di- hydro-5-oxa-1,2a-diaza- acenaphthylene-7-carboxylic acid methyl ester | 660 mg, white solid, 65% |

| | |
|---|---|
| NMR | 7.9(s, 1H), 7.37(s, 1H), 5.17(s, 2H), 4.28(s, 2H), 3.86(s, 3H), 1.65(s, 6H) |

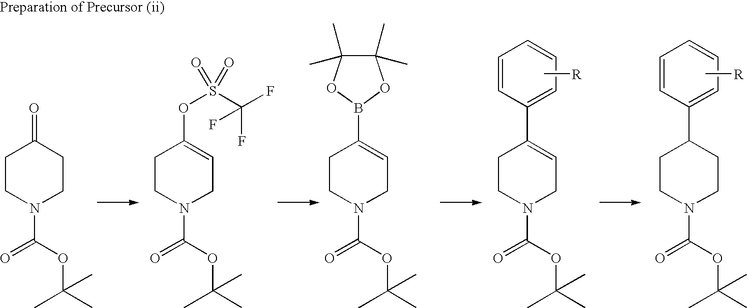

### EXAMPLE 15.1

### Tert-butyl-4-{[(trifluoromethyl)sulfonyl]oxy}-3,6-dihydropyridine-1(2H)-carboxylate

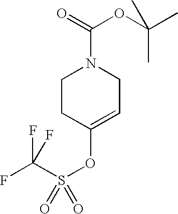

To a solution of N,N-diisopropyl amine (4.2 ml, 30 mmol) in anhydrous THF (130 ml), the solution of n-BuLi in pentane (15 ml, 30 mmol) was added in a dropwise fashion at 0° C. 15 min later, the solution of tert-butyl-4-oxopiperidine-1-carboxylate (4.98 g, 25 mmol) in anhydrous (60 ml) was added to the reaction at −78° C. in a dropwise fashion. 30 min later, a solution of 2,2,2-trifluoro-N-phenyl-N-[(trifluoromethylsulfonyl]ethanesulfonamide (9.8 g, 27.5 mmol) was added to the reaction mixture. After 1 h., the reaction was allowed to warm up to room temperature and stirred 3 h. The reaction mixture was quenched with saturated sodium bicarbonate solution (100 ml), and the product was extracted with EtOAc. The organic layer was washed with water, a brine solution, dried over anhydrous Na₂SO₄, filtered, and concentrated in vacuo. Purification by flash chromatography (silica gel, gradient elution with 5% to 10% ethyl acetate/hexanes) provided off-white solid (5.88 g, 75%). ¹H NMR (300 MHz, CDCL₃): 5.70 (br, 1H), 4.00 (br, 2H), 3.58 (t, 2H), 2.39 (br, 2H), 1.42 (s, 9H).

### EXAMPLE 16.1

### Tert-butyl-4-[(1,1,2,2,-tetramethyl)-boronate ester]-3,6-dihydropyridine-1(2 H)-carboxylate

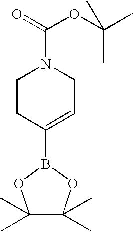

To a solution of tert-butyl-4-{[(trifluoromethyl)sulfonyl]oxy}-3,6-dihydropyridine-1(2H)-carboxylate (5.88 g, 18.65 mmol) in dioxane (60 ml), bis(pinacolate)diboron (5.16 g, 20.51 mmol), [1,1′-bis(diphenylphosphino)-ferrocene]dichloropalladium (910 mg, 1.12 mmol) and sodium acetate (4.6 g, 55.95 mmol) were added. The resulting mixture was stirred at 80° C. overnight. After removal of solvent, the residue was partitioned between EtOAc and water. The organic layer was washed with water, a brine solution, dried over anhydrous Na₂—SO₄, filtered, and concentrated in vacuo. Purification by flash chromatography (silica gel, gradient elution with 10% to 20% ethyl acetate/hexanes) provided off-white solid (1.75 g, 35%). ¹H NMR (300 MHz, CDCL₃): 6.40 (br, 1H), 3.89 (br, 2H), 3.77 (t, 2H), 2.17 (br, 2H), 1.40 (s, 9H), 1.20 (s, 12H).

### EXAMPLE 17.1

### Tert-Butyl 4-(4-fluorophenyl)-3,6-dihydropyridine-1(2H)-carboxylate

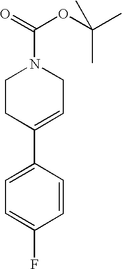

The solution of 1-fluoro-4-iodobenzene (463.2 mg, 0.0.74 mmol) in DMF (15 ml) was degassed, and back-filled with Argon. Tert-butyl-4-[(1,1,2,2,-tetramethyl)-boronate ester]-3,6-dihydropyridine-1(2H)-carboxylate (250 mg, 0.81 mmol), [1,1′-bis(diphenylphosphino)-ferrocene]dichloropalladium (60 mg, 0.074 mmol) and potassium carbonate (305 mg, 2.2 mmol)) were added to the solution. The resulting mixture was stirred over night at 110° C., then was poured into water and extracted three times with ethyl acetate. The combined organic layers were washed with a brine solution, dried over anhydrous Na₂SO₄, filtered, and concentrated in vacuo. Purification by flash chromatography (silica gel, gradient elution with 10% to 20% ethyl acetate/hexanes) provided a dark-green oil (153 mg, 75%). ¹H NMR (300 MHz, CDCL₃): 7.32-7.35 (m, 2H), 6.99-7.05 (m, 2H), 5.90 (br, 1H), 4.07 (br, 2H), 3.64 (t, 2H), 2.50 (br, 2H), 1.51 (s, 9H).

In a similar fashion, the following compounds were made:

| Ex- ample 17.2 | 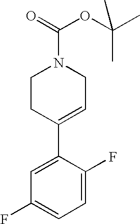 | Tert-Butyl 4-(2,5-di- fluorophenyl)-3,6-di- hydro- pyridine-1(2H)-carboxy- late | 153 mg (71%), green oil |
|---|---|---|---|
| NMR | 7.17-7.22(m, 1H), 6.79-6.83(m, 2H), 5.90(br, 1H), | | |
| | 4.06(br, 2H), 3.62(t, 2H), 2.47(br, 2H), 1.50(s, 9H). | | |
| Ex- ample 17.3 | 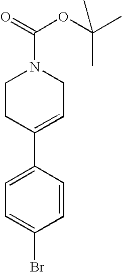 | Tert-Butyl 4-(4-bromo- phenyl)-3,6-di- hydro- pyridine-1(2H)-carboxy- late | 300 mg (75%), green oil |
| NMR | 7.44-7.47(m, 2H), 7.23-7.26(m, 2H), 6.00(br, 1H), | | |
| | 4.15(br, 2H), 3.68(t, 2H), 2.50(br, 2H), 1.51(s, 9H). | | |
| Ex- ample 17.4 | 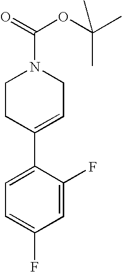 | Tert-Butyl 4-(2,4-di- fluorophenyl)-3,6-di- hydro- pyridine-1(2H)-carboxy- late | 980 mg (72%), green oil |
| NMR | 7.08-7.19(m, 1H), 6.70-6.75(m, 2H), 5.81(br, 1H), | | |
| | 3.99(br, 2H), 3.55(t, 2H), 2.40(br, 2H), 1.43(s, 9H). | | |
| Ex- ample 17.5 | 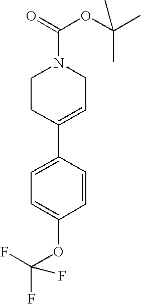 | Tert-Butyl 4-(4-tri- fluoro- methoxyphenyl)-3,6-di- hydro- pyridine-1(2H)-carboxy- late | 310 mg (77%), green oil |
| NMR | 7.37-7.40(m, 2H), 7.17-7.20(m, 2H), 6.00(br, 1H), | | |
| | 4.15(br, 2H), 3.68(t, 2H), 2.50(br, 2H), 1.50(s, 9H). | | |
| Ex- ample 17.6 | 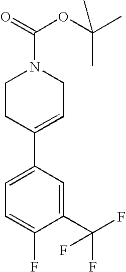 | Tert-Butyl 4-[4-fluoro-3-(tri- fluoro- methyl)phenyl]-3,6-di- hydro- pyridine-1(2H)-carboxy- late | 169 mg (60%), yellow gum |
| NMR | 7.49-7.57(m, 2H), 7.14(t, 1H), 6.03(br, 1H), | | |
| | 4.07(br, 2H), 3.64(t, 2H), 2.48(br, 2H), 1.48(s, 9H). | | |
| Ex- ample 17.7 | 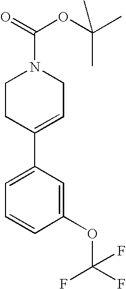 | Tert-Butyl 4-(3-tri- fluoro- methoxyphenyl)-3,6-di- hydro- pyridine-1(2H)-carboxy- late | 122 mg (44%), green oil |
| NMR | 7.31-7.36(m, 2H), 7.21(s, 1H), 7.02-7.10(d, 2H), 6.10(br, | | |
| | 1H), 4.10(br, 2H), 3.64(t, 2H), 2.50(br, 2H), 1.50(s, 9H). | | |
| Ex- ample 17.8 | 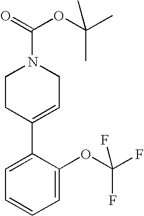 | Tert-Butyl 4-(2-tri- fluoro- methoxyphenyl)-3,6-di- hydro- pyridine-1(2H)-carboxy- late | 174 mg (63%), green oil |

| | |
|---|---|
| NMR | 7.20-7.30(m, 4H), 5.80(br, 1H), 4.06(br, 2H), |
| | 3.61(t, 2H), 2.50(br, 2H), 1.51(s, 9H). |

### EXAMPLE 18.1

### 4-(2,5-difluorophenyl)-piperidine

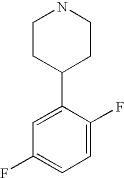

To a solution of tert-Butyl 4-(2,5.di-fluorophenyl)-3,6-dihydropyridine-1(2H)-carboxylate (53 mg, 0.179 mmol), palladium oxide (20 mg) was added, followed by charging a hydrogen-filled balloon on the top of flask. The reaction was stirred at room temperature overnight. The reaction mixture was filtered through diatomaceous earth and filtrate was condensed to get a colorless oil, which was further stirred in TFA/DCM (2 ml, v/v=1:1) overnight. Removal of solvents provided a yellow gum (40 mg, 81%), confirmed with GC/MS.

In a similar fashion, the following compounds were made:

| | | | |
|---|---|---|---|
| Example 18.2 | 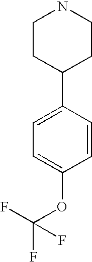 | 4-[4- (trifluoromethoxy)phenyl]- piperidine | 120 mg (85%), yellow gum |
| Example 18.3 | 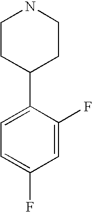 | 4-(2,4-difluorophenyl)- piperidine | 600 mg (90%), yellow gum |
| Example 18.4 | 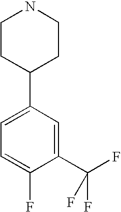 | 4-[4-fluoro-3- (trifluoromethyl)phenyl]- piperidine | 120 mg (81%), yellow gum |
| Example 18.5 | 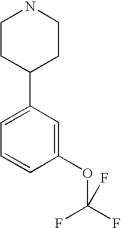 | 4-[3- (trifluoromethoxy)phenyl]- piperidine | 80 mg (90%), yellow gum |
| Example 18.6 | 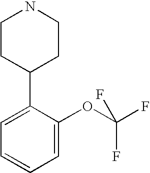 | 4-[2- (trifluoromethoxy)phenyl]- piperidine | 85 mg (88%), yellow gum |

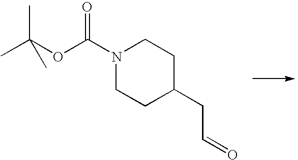

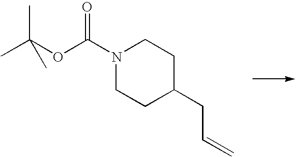

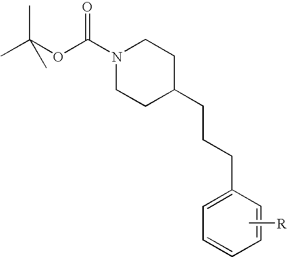

### EXAMPLE 19.1

### Tert-butyl-4-allylpiperidine-1-carboxylate

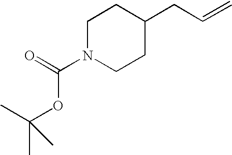

To a solution of tert-butyl-4-(2-oxoethyl)piperidine-1-carboxylate (2.13 g, 9.37 mmol) in acetonitrile (30 ml), 1,8-diazabicyclo[5,4,0]undec-7-ene (2.85 g, 18.74 mmol) and methyltriphenyl phosphonium bromide (6.69 g, 18.74 mmol) were added. The reaction was refluxed overnight and diluted with EtOAc, washed with water, brine, dried over anhydrous Na₂SO₄, filtered, and concentrated in vacuo. Purification by flash chromatography (silica gel, gradient elution with 10% to 20% ethyl acetate/hexanes) provided a yellow oil (1.91 g, 91%). ¹H NMR (300 MHz, CDCL₃): 5.62-5.80 (m, 1H), 4.94-4.99 (m, 1H), 4.93 (s, 1H), 4.07 (br, 2H), 2.62 (br, 2H), 1.96 (t, 2H), 1.59-1.63 (m, 2H), 1.41 (s, 9H), 1.08-1.12 (m, 1H), 0.96-1.06 (m, 2H).

In a similar fashion, the following compounds were made:

| | | | |
|---|---|---|---|
| Example 19.2 | 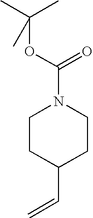 | Tert-butyl-4- vinylpiperidine-1- carboxylate | 2.2 g (75%), yellow oil |

### EXAMPLE 20.1

### Tert-butyl-4-[3-(4-fluorophenyl)propyl]piperidine-1-carboxylate

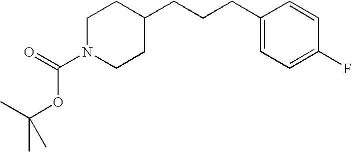

Tert-butyl-4-allylpiperidine-1-carboxylate (500 mg, 2.22 mmol) was degassed and back-filled with Argon. 9-BBN (0.5 M in THF, 4.44 ml, 2.66 mmol) was added through a syringe. The mixture was stirred at 60° C. for 1 h. After cooled to room temperature, it was added to the mixture of 1-bromo-4-fluorobenzene (466 mg, 2.66 mmol), potassium carbonate (460 mg, 3.33 mmol), and [1,1′-bis(diphenylphosphino)-ferrocene]dichloropalladium (54 mg, 0.067 mmol) in DMF (10 ml) and water (0.1 ml). The resulting mixture was stirred at 85° C. for 40 h. After cooled down to room temperature, it was diluted with water and the product was extracted with EtOAc three times. The combined organic layers were washed with water, brine, dried over anhydrous Na₂SO₄, filtered, and concentrated in vacuo. Purification by flash chromatography (silica gel, gradient elution with 10% to 20% ethyl acetate/hexanes) provided a yellow oil (520 mg, 73%). ¹H NMR (300 MHz, CDCL₃): 7.07-7.12 (m, 2H), 6.90-6.96 (m, 2H), 4.07 (br, 2H), 2.52-2.70 (m, 4H), 156-1.64 (m, 4H), 1.44 (s, 9H), 1.19-1.39 (m, 3H), 086-1.06 (m, 2H).

In a similar fashion, the following compounds were made:

| Example 20.2 | 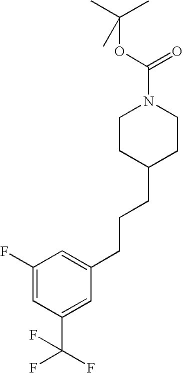 | Tert-butyl-4-[3-(3- fluoro-5- trifluoromethyl- phenyl)propyl]- piperidine- 1-carboxylate | 790 mg (91%), yellow oil |
|---|---|---|---|
| NMR | 7.19 (m, 1H), 7.00-7.15 (m, 2H), 4.07 (br, 2H), | | |
| | 2.59-2.70 (m, 4H), 149-1.64 (m, 4H), 1.42 (s, 9H), | | |
| | 1.24-1.34 (m, 3H), 086-1.06 (m, 2H). | | |
| Example 20.3 | 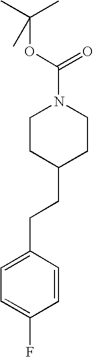 | Tert-butyl-4-[2-(4- fluorophenyl)ethyl]- piperidine-1- carboxylate | 2.65 g (89%), yellow oil |

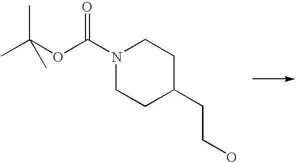

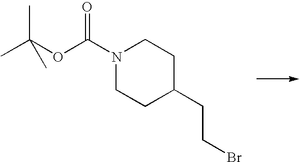

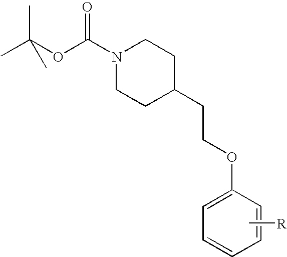

### EXAMPLE 21.1

### Tert-butyl-4-(2-bromoethyl)piperidine-1-carboxylate

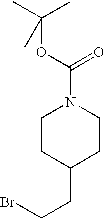

To a solution of tert-butyl-4-(2-hydroxyethyl)piperidine-1-carboxylate (1.5 g, 6.54 mmol) and tetrabromide carbon (3.25 g, 9.81 mmol) in DCM (20 ml), the solution of triphenylphosphine (1.72 g, 6.54 mmol) was added slowly. The reaction was stirred overnight and diluted with hexanes (50 ml), washed with water, brine, dried over anhydrous Na₂SO₄, filtered, and concentrated in vacuo. Purification by flash chromatography (silica gel, gradient elution with 10 to 30% ether/hexanes) provided a yellow oil (1.67 g, 88%). ¹H NMR (300 MHz, CDCL₃): 4.07 (br, 2H), 3.35 (br, 2H), 2.60 (br, 2H), 1.69-1.72 (m, 2H), 1.56-1.60(m, 3H), 1.36 (s, 9H), 0.86-1.06 (m, 2H).

### EXAMPLE 22.1

### Tert-butyl-4-[2-(4-fluorophenoxy)ethyl]piperidine-1-carboxylate

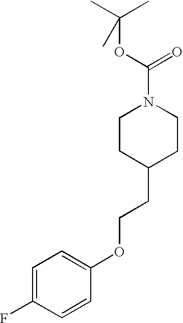

To a solution of tert-butyl-4-(2-bromoethyl)piperidine-1carboxylate (600 mg, 2.05 mmol) in acetone (30 ml), 4-fluorophenol (230 mg, 2.05 mmol), potassium carbonate (1.12 g, 8.2 mmol) and tetrabutylammonium iodide (45 mg, 0.123 mmol) was added. The resulting mixture was refluxed overnight. After removal of acetone, the residue was partitioned between water and water. The organic layer was washed with 1N NaOH three times, water, brine, dried over anhydrous Na₂SO₄, filtered, and concentrated in vacuo to provide a yellow oil (700 mg, 98%). ¹H NMR (300 MHz, CDCL₃): 6.89-6.94 (m, 2H), 6.75-6.80 (m, 2H), 4.07 (br, 2H), 3.91 (t, 2H), 2.61 (br, 2H), 165-1.68 (m, 5H), 1.43 (s, 9H), 1.02-1.18 (m, 2H).

In a similar fashion, the following compounds were made:

| Example 22.2 | 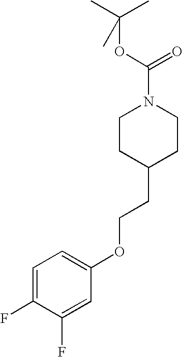 | Tert-butyl-4-[2-(3,4- difluorophenoxy)- ethyl]piperidine- 1-carboxylate | 328 mg (94%), yellow oil |
|---|---|---|---|
| NMR | 7.03-7.06 (q, 1H), 6.65-6.90 (m, 1H), 6.50-6.60 (m, 1H), | | |
| | 4.10 (br, 2H), 3.95 (t, 2H), 2.75 (br, 2H), 169-1.73 (m, | | |
| | 5H), 1.46 (s, 9H), 1.05-1.18 (m, 2H). | | |
| Example 22.3 | 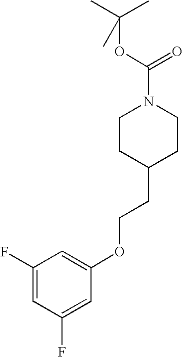 | Tert-butyl-4-[2-(3,5- difluorophenoxy)- ethyl]piperidine- 1-carboxylate | 317 mg (99%), yellow oil |

| | |
|---|---|
| NMR | 6.38-6.42 (m, 3H), 4.10 (br, 2H), 3.96 (t, 2H), 2.70 (br, |
| | 2H), 168-1.73 (m, 5H), 1.46 (s, 9H), 1.05-1.15 (m, 2H). |

### EXAMPLE 23.1

### 6-Fluoro-8-nitro-2,3-dihydro-1H-quinolin-4-one

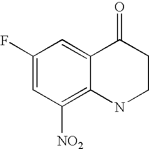

### (i) 3-[(4-fluoro-2-nitrophenyl)amino]propanenitrile

To a stirring solution of (4-fluoro-2-nitrophenyl)amine (10 g, 64.1 mmol) in 1,4-dioxane was added acrylonitrile (6.23 mL, 96.1 mmol) and 40% Triton B in water (0.5 mL). The mixture stirred at room temperature overnight and was concentrated. The resulting black solid was dried under vacuum for 3 h., suspended in ether, and stirred vigorously for another 4 h. The suspension was filtered, washed with ether and dried under vacuum to afford 8.47 g (63%) of the title compound as a brown solid. ¹H NMR (300 MHz, CDCl₃): δ 8.08 (br s, 1H), 7.99-7.95 (dd, 1H), 7.37-7.31 (m, 1H), 6.89-6.84 (dd, 1H), 3.77-3.71 (q, 2H), 2.79-2.75 (t, 2H).

### (ii) 3-[(4-fluoro-2-nitrophenyl)amino]propanoic acid

3-[(4-fluoro-2-nitrophenyl)amino]propanenitrile (5.72 g, 27.3 mmol) was suspended in methanol (50 mL) and 10% sodium hydroxide (50 mL) was added. The mixture refluxed for 2 h., was cooled to room temperature and was concentrated. The resulting slurry was diluted with water (100 mL) and was acidified with 10% hydrochloric acid 100 mL) to pH ˜1. The resulting suspension was filtered and washed with water. The resulting aqueous washes were combined, re-acidified, filtered and washed with water. The precipitate was combined and dried under vacuum providing the title compound as an orange solid (4.90 g, 79%). ¹H NMR (300 MHz, CDCl₃): δ 8.10 (br s, 1H), 7.79-7.91 (m, 1H), 7.34-7.30 (m, 1H), 6.91-6.87 (dd, 1H), 3.69-3.67 (m, 2H), 2.82-2.78 (t, 2H).

### (iii) 6-fluoro-8-nitro-2,3-dihydroquinolin-4(1H)-one

To a flask stirring with Eatons Reagent (11 mL) was added 3-[(4-fluoro-2-nitrophenyl)amino]propanoic acid (0.65 g, 2.85 mmol). The mixture stirred at 60° C. for 3.5 h. and was then cooled to room temperature. Ice chips were added and the mixture was poured into water. The suspension was filtered and the precipitate was washed with water and dried under vacuum. Purification by column chromatography (silica gel, gradient elution with 5% to 25% ethyl acetate/hexanes) provided the title compound as an orange solid (0.342 g, 57%). ¹H NTMR (300 MHz, CDCl₃): δ: 8.19 (br s, 1H), 8.17-8.13 (dd, 1H), 8.00-8.97 (dd, 1H), 3.84-3.78 (m, 2H), 3.87-3.82 (m, 2H).

### EXAMPLE 24.1

### 8-Amino-6-fluoro-2,3-dihydro-1H-quinolin-4-one

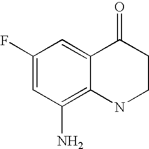

A solution of 6-Fluoro-8-nitro-2,3-dihydro-1H-quinolin-4-one in ethyl acetate (25 mL) and acetic acid (10 mL) was purged with argon and 10% palladium on carbon was added (200 mg). This mixture was stirred for 18 h. at room temperature under hydrogen and was filtered through diatomaceous earth concentrated in vacuo. The crude product was used directly in the next step. ¹H NMR (300 MHz, CDCL₃): 7.13 (dd, 1H), 6.65 (dd, 1H), 3.59 (dd, 2H), 2.72 (dd, 2H).

### EXAMPLE 25.1

### 2-Chloromethyl-8-fluoro-6,6-dimethoxy-5,6-dihydro-4H-imidazo[4,5,1-ij]quinoline

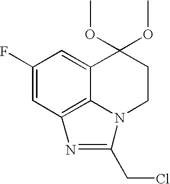

To 8-Amino-6-fluoro-2,3-dihydro-1H-quinolin-4-one was added 2-chloro-1,1,1-triethoxy ethane (10 mL) and concentrated hydrochloric acid (0.5 mL). The reaction was stirred for 0.5 h. and diluted with dichloromethane (25 ml), and quenched with saturated sodium bicarbonate. The organics were washed with water and brine, dried over anhydrous sodium sulfate, filtered, and concentrated in vacuo. Purification by flash chromatography (silica gel, gradient elution with 30 to 60% ethyl acetate/hexanes) and trituration with hexanes afforded a yellow solid (0.722 g, 53% over 2 steps). ¹H NMR (300 MHz, CDCL₃): 7.38 (dd, 1H), 7.22 (dd, 1H), 4.84 (s, 2H), 4.39 (dd, 2H), 3.33 (s, 6H), 2.43 (dd, 2H).

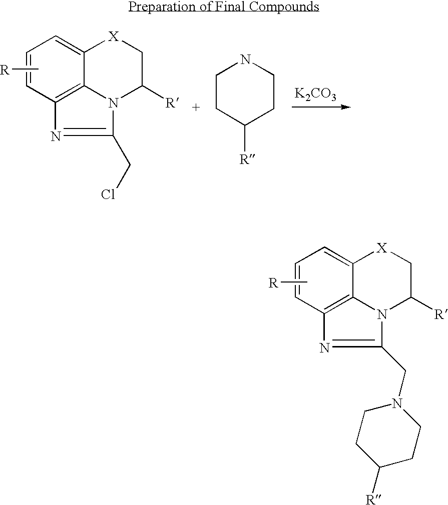

### EXAMPLE 26.1

### 8-fluoro-4-methyl-2-[(4-phenylpiperidin-1-yl)methyl]-5,6-dihydro-4H-imidazo[4,5,1-ij]quinoline

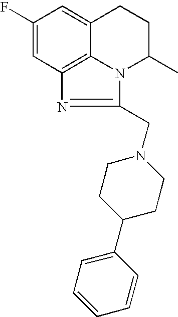

To a solution of 2-(chloromethyl)-8-fluoro-4-methyl-5,6-dihydro-4H-imidazo[4,5,1-ij]quinoline (23.8 mg, 0.114 mmol) in acetonitrile (5 ml), 4-phenylpiperidine (27.6 mg, 0.17 mmol) and potassium carbonate (79 mg, 0.57 mmol) were added. The resulting mixture was stirred overnight. Then, the resulting reaction mixture was diluted with water and the product was extracted with EtOAc. The aqueous layer was back-extracted with EtOAc, and the combined organic layers were washed with brine, dried over anhydrous Na₂SO₄, filtered, and concentrated in vacuo. Purification by flash chromatography (silica gel, gradient elution with 80% ethyl acetate/hexanes to 5% 2M NH₃ in methanol/(80% ethyl acetate/hexanes)) provided a brown foam (34.7 mg, 84%). ¹H NMR (300 MHz, CDCl₃): δ 7.21-7.35 (m, 6H), 6.81-6.84 (m, 1H), 4.97-4.99 (m, 1H), 3.85 (s, 2H) 2.94-3.09 (m, 4H), 2.29-2.41 (m, 1H), 2.16-2.32 (m, 4H), 1.65-1.86 (m, 3H), 1.50 (d, 3H), 1.15-1.32 (m, 1H).

In a similar fashion, the following compounds were made:

| Example | Structure | Name | Yield |
|---|---|---|---|
| 26.2 | 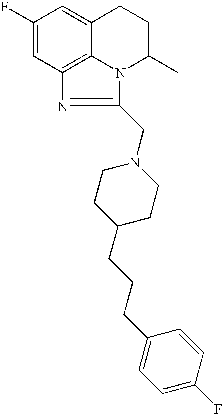 | 8-fluoro-4-methyl-2-{4- [3-(4-fluorophenyl) propyl]piperidin-1- ylmethyl}-5,6-dihydro- 4H-imidazo[4,5,1- ij]quinoline | 18 mg (37%) brown oil |
| NMR | 7.23 (dxd, 1H), 7.10-7.20 (m, 2H), 6.93-6.99 (m, 2H), 6.79 (d, 1H), 4.92-4.94 (m, 1H), | | |
| | 3.77 (s, 2H), 2.86-3.15 (m, 4H), 2.56 (t, 2H), 2.07-2.15 (m, 4H), 1.60-1.75 (m, 4H), | | |
| | 1.45 (d, 3H), 1.21-1.29 (m, 5H) | | |
| 26.3 | 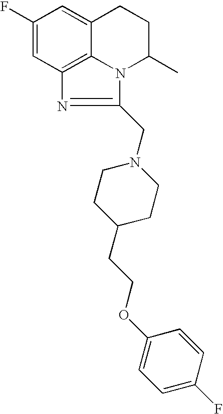 | 8-fluoro-4-methyl-2-{4- [2-(4-fluorophenoxy) ethyl]piperidin-1- ylmethyl}-5,6-dihydro- 4H-imidazo[4,5,1- ij]quinoline | 26.9 mg (56%), brown oil |
| NMR | 7.23 (dxd, 1H), 6.97-7.00 (m, 2H), 6.80-6.85 (m, 3H), 4.92-4.95 (m, 1H), 3.96 (t, 3H), | | |
| | 3.79 (s, 2H), 2.85-3.12 (m, 4H), 2.13-2.17 (m, 2H), 1.69-1.76 (m, 3H), 1.30-1.40 (m, | | |
| | 1H), 1.46 (d, 3H), 1.22-1.33 (m, 3H) | | |
| 26.4 | 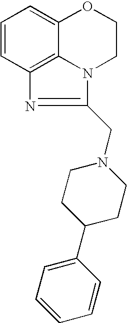 | 2-[(4-phenylpiperidin-1- yl)methyl]-4,5- dihydroimidazo[1,5,4- de][1,4]benzoxazine | 36.8 mg (92%), yellow solid |
| NMR | 7.22-7.35 (m, 6H), 7.13 (t, 1H), 6.75 (d, 1H), 4.52 (s, 4H), 3.91 (s, 2H), 2.98-3.02 (m, | | |
| | 2H), 2.48-2.62 (m, 1H), 2.28-2.33 (m, 2H), 1.73-1.86 (m, 4H) | | |
| 26.5 | 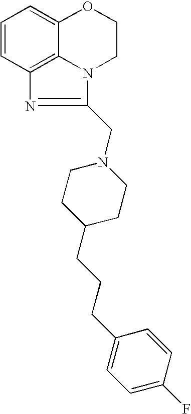 | 2-{4-[3-(4-fluorophenyl) propyl] piperidin-1- ylmethyl}-4,5- dihydroimidazo[1,5,4- de][1,4]benzoxazine | 48 mg (100%), yellow oil |
| NMR | 7.32 (d, 1H), 7.09-7.15 (m, 3H), 6.94-7.00 (m, 2H), 6.72 (d, 1H), 4.48 (s, 4H), 3.83 (s, | | |
| | 2H), 2.82-2.86 (m, 2H), 2.57 (t, 3H), 2.07-2.10 (m, 2H), 1.60-1.70 (m, 4H), 1.24-1.29 | | |
| | (m, 5H) | | |
| 26.6 | 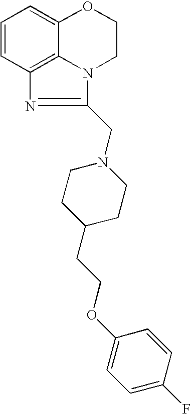 | 2-{4-[2-(4- fluorophenoxy) ethyl]piperidin-1- ylmethyl}-4,5- dihydroimidazo[1,5,4- de][1,4]benzoxazine | 50.4 mg (100%), yellow solid |
| NMR | 7.30-7.33 (m, 1H), 7.12 (t, 1H), 6.94-6.97 (m, 2H), 6.75-6.85 (m, 2H), 6.72 (d, 1H), | | |
| | 4.48 (s, 4H), 3.96 (t, 2H), 3.84 (s, 2H), 2.85-2.88 (m, 2H), 2.15-2.19 (m, 2H), 1.69-1.77 | | |
| | (m, 4H), 1.38-1.60 (m, 1H), 1.25-1.31 (2H) | | |
| 26.7 | 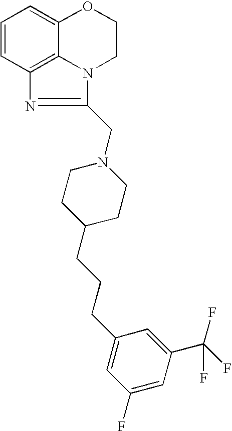 | 2-{4-[3-(3-fluoro- 5(trifluoromethyl)- phenyl) propyl] piperidin- 1-ylmethyl}-4,5- dihydroimidazo[1,5,4- de][1,4]benzoxazine | 64 mg, (100%), yellow oil |
| NMR | 7.30-7.32 (m, 1H), 7.23 (s, 1H), 7.05-7.17 (m, 3H), 6.73 (d, 1H), 4.47 (s, 2H), 3.83 (s, | | |
| | 2H), 2.83-2.86 (m, 2H), 2.65 (t, 2H), 2.06-2.15 (m, 2H), 1.62-1.70 (m, 2H), 1.19-1.30 | | |
| | (m, 5H) | | |
| 26.8 | 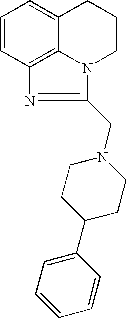 | 2-[(4-phenylpiperidin-1- yl)methyl]-5,6-dihydro- 4H-imidazo[4,5,1- ij]quinoline | 36.3 mg (100%), brown solid |
| NMR | 7.57 (d, 1H), 7.15-7.32 (m, 6H), 7.03 (d, 1H), 4.33-4.37 (m, 2H), 3.88 (s, 2H), 3.00-3.04 | | |
| | (m, 4H), 2.68-2.80 (m, 1H), 2.24-2.33 (m, 1H), 1.73-1.84 (m, 4H) | | |
| 26.9 | 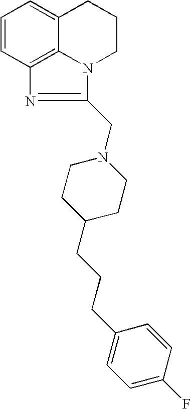 | 2-{4-[3-(4-fluorophenyl) propyl] piperidin-1- ylmethyl}-5,6-dihydro- 4H-imidazo[4,5,1- ij]quinoline | 35.5 mg (94%), brown solid |
| NMR | 7.54 (dxd), 1H), 7.10-7.18 (m, 3H), 6.94-7.02 (m, 3H), 4.27-4.31 (m, 2H), 3.80 (s, | | |
| | 2H), 3.00 (t, 2H), 2.83-2.87 (m, 2H), 2.57 (t, 2H), 2.06-2.56 (m, 5H), 1.57-1.68 (m, | | |
| | 4H), 1.18-1.28 (m, 4H) | | |
| 26.10 | 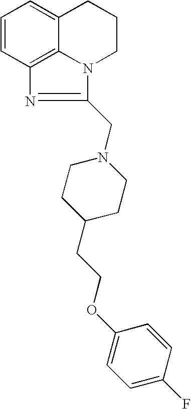 | 2-{4-[2-(4- fluorophenoxy) ethyl]piperidin-1- ylmethyl}-5,6-dihydro- 4H-imidazo[4,5,1- ij]quinoline | 31.5 mg (83%), brown solid |
| NMR | 7.55 (d, 1H), 7.06 (t, 1H), 6.94-7.02 (m, 3H), 6.80-6.85 (m, 2H), 4.28-4.32 (m, 2H), | | |
| | 3.96 (t, 2H), 3.81 (s, 2H), 3.00 (t, 2H), 2.86-2.90 (m, 2H), 2.10-2.29 (m, 4H), 1.68-1.75 | | |
| | (m, 4H), 1.50-1.65 (m, 1H), 1.26-1.34 (m, 2H) | | |
| 26.11 | 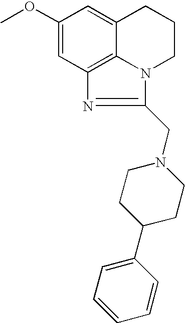 | 8-methoxy-2-[(4- phenylpiperidin-1- yl)methyl]-5,6-dihydro- 4H-imidazo[4,5,1- ij]quinoline | 8.0 mg (35%), brown oil |
| NMR | 7.21-7.32 (m, 5H), 7.05 (s, 1H), 6.71 (s, 1H), 4.29-4.33 (m, 2H), 3.85-3.86 (m, 5H), | | |
| | 2.95-2.99 (m, 4H), 2.23-2.28 (m, 4H), 1.85-1.95 (m, 5H) | | |
| 26.12 | 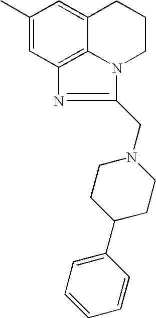 | 8-methyl-2-[(4- phenylpiperidin-1- yl)methyl]-5,6-dihydro- 4H-imidazo[4,5,1- ij]quinoline | 32.5 mg (70%), yellow oil |
| NMR | 7.21-7.35 (m, 6H), 6.87 (s, 1H), 4.30-4.34 (m, 2H), 3.86 (s, 2H), 2.95-3.02 (m, 4H), | | |
| | 2.49-2.65 (m, 4H), 2.24-2.31 (m, 4H), 1.73-1.84 (m, 4H) | | |
| 26.13 | 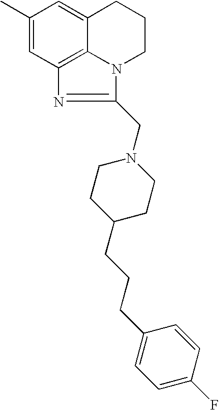 | 8-methyl-2-{4-[3-(4- fluorophenyl) propyl]piperidin-1-ylmethyl}- 5,6-dihydro-4H- imidazo[4,5,1-ij]quinoline | 47.2 mg (86%), yellow oil |
| NMR | 7.33 (s, 1H), 7.10-7.15 (m, 2H), 6.93-6.99 (m, 2H), 6.85 (s, 1H), 4.24-4.28 (m, 2H), | | |
| | 3.78 (s, 2H), 2.95 (t, 2H), 2.82-2.86 (m, 2H), 2.56 (t, 2H), 2.47 (s, 3H), 2.05-2.23 (m, | | |
| | 5H), 1.57-1.67 (m, 4H), 1.15-1.27 (m, 4H) | | |
| 26.14 | 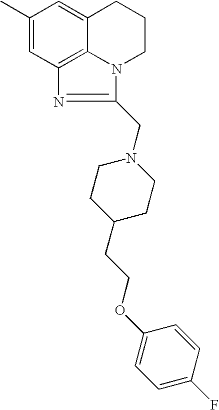 | 8-methyl-2-{4-[2-(4- fluorophenoxy) ethyl]piperidin-1- ylmethyl}-5,6-dihydro- 4H-imidazo[4,5,1- ij]quinoline | 44.9 mg (82%), yellow solid |
| NMR | 7.33 (s, 1H), 6.94-7.00 (m, 2H0, 6.80-6.86 (m, 3H0, 4.25-4.29 (m, 2H), 3.95 (t, 2H), | | |
| | 3.79 (s, 2H), 2.95 (t, 2H), 2.85-2.89 (m, 2H), 2.48 (s, 3H), 2.09-2.24 (m, 4H), 1.68-1.74 | | |
| | (m, 4H), 1.50-1.65 (m, 1H), 1.25-1.30 (m,2H) | | |
| 26.15 | 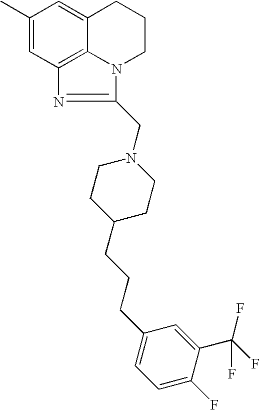 | 8-methyl-2-{4-[3-(3- fluoro-5(trifluoromethyl)- phenyl) propyl] piperidin- 1-ylmethyl}-5,6-dihydro- 4H-imidazo[4,5,1- ij]quinoline | 65.9 mg (100%), yellow oil |
| NMR | 7.30 (s, 1H), 7.23 (s, 1H), 7.05-7.16 (m, 2H), 6.85 (s, 1H), 4.24-4.28 (m, 2H), 3.78 (s, | | |
| | 2H), 2.95 (t, 2H), 2.83-2.87 (m, 2H), 2.65 (t, 2H), 2.47 (s, 2H), 2.05-2.28 (m, 5H), | | |
| | 1.64-1.67 (m, 4H), 1.19-1.29 (m, 4H) | | |
| 26.16 | 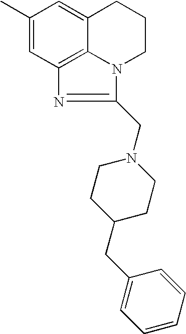 | 8-methyl-2-[(4- benzylpiperidin-1- yl)methyl]-5,6-dihydro- 4H-imidazo[4,5,1- ij]quinoline | 59 mg (100%), yellow oil |
| NMR | 7.13-7.33 (m, 6H), 6.86 (s, 1H), 4.25-4.29 (m, 2H), 3.78 (s, 2H), 2.95 (t, 2H), 2.83-2.87 | | |
| | (m, 2H), 2.42-2.55 (m, 5H), 2.19-2.24 (, m, 2H), 2.04-2.11 (m, 2H), 1.45-1.65 (m, | | |
| | 3H), 1.24-1.29 (m, 2H) | | |
| 26.17 | 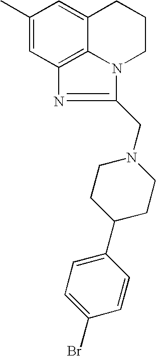 | 8-methyl-2-{[4-(4- bromophenyl)piperidin-1- yl]methyl}-5,6-dihydro- 4H-imidazo[4,5,1- ij]quinoline | 36.1 mg (95%), yellow solid |
| NMR | 7.40-7.43 (m, 2H), 7.34 (s, 1H), 7.08-7.12 (m, 2H), 6.87 (s, 1H), 4.28-4.32 (m, 2H), | | |
| | 3.85 (s, 2H), 2.95-3.01 (m, 4H), 2.40-2.55 (m, 4H), 2.20-2.30 (m, 4H), 1.48-1.83 (m, | | |
| | 4H) | | |
| 26.18 | 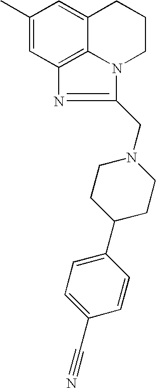 | 8-methyl-2-{[4-(4- cyanophenyl)piperidin-1- yl]methyl}-5,6-dihydro- 4H-imidazo[4,5,1- ij]quinoline | 26.3 mg (77%), yellow solid |
| NMR | 7.57-7.61 (m, 2H), 7.31-7.34 (m, 3H), 6.87 (s, 1H), 4.27-4.31 (m, 2H), 3.85 (s, 2H), | | |
| | 2.95-3.04 (m, 4H), 2.52-2.62 (m, 1H), 2.48 (s, 2H), 2.23-2.31 (m, 4H), 1.70-1.85 (m, | | |
| | 4H) | | |
| 26.19 | 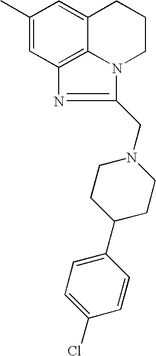 | 8-methyl-2-{[4-(4- chlorophenyl)piperidin-1- yl]methyl}-5,6-dihydro- 4H-imidazo[4,5,1- ij]quinoline | 27.6 mg (81%), yellow solid |
| NMR | 7.34 (s, 1H0, 7.25-7.28 (m, 2H), 7.14-7.18 (m, 2H), 6.87 (s, 1H), 4.28-4.32 (m, 2H), | | |
| | 3.85 (s, 2H), 2.95-3.01 (m, 4H), 2.48-2.60 (m, 4H), 2.19-2.30 (m, 4H), 1.67-1.80 (m, | | |
| | 4H) | | |
| 26.20 | 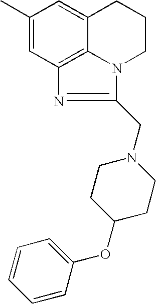 | 8-methyl-2-[(4- phenoxypiperidin-1- yl)methyl]-5,6-dihydro- 4H-imidazo[4,5,1- ij]quinoline | 25.5 mg (78%), yellow oil |
| NMR | 7.26-7.34 (m, 3H), 6.87-6.97 (m, 4H), 4.27-4.34 (m, 4H), 3.84 (s, 2H), 2.96 (t, 2H), | | |
| | 2.79-2.85 (m, 2H), 2.78 (s, 3H), 2.35-2.43 (m, 2H), 2.22-2.26 (m, 2H), 1.95-2.07 (m, | | |
| | 2H), 1.78-1.82 (m, 2H) | | |
| 26.21 | 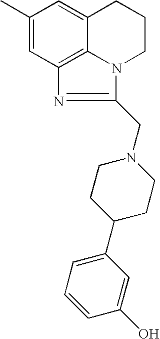 | 8-methyl-2-{[4-(3- hydroxyphenyl)piperidin- 1-yl]methyl}-5,6-dihydro- 4H-imidazo[4,5,1- ij]quinoline | 16 mg (63%), yellow solid |
| NMR | 7.38 (s, 1H), 7.19-7.281 (m, 1H), 6.89-6.92 (m, 2H), 6.73-6.82 (m, 2H), 4.33-4.36 (m, | | |
| | 2H), 3.86 (s, 2H), 2.96-3.01 (m, 4H), 2.45 (s, 3H), 2.17-2.28 (m, 5H), 1.70-1.81 (m, | | |
| | 4H) | | |
| 26.22 | 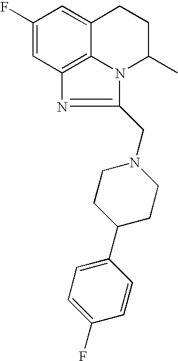 | 8-fluoro-4-methyl-2-[4- (4-fluoro-phenyl)- piperidin-1-ylmethyl]-5,6- dihydro-4H- imidazo[4,5,1-ij]quinoline | 55.3 mg (81.4%), brown solid |
| NMR | 7.16-7.20 (m, 3H), 6.95-7.03 (m, 2H), 6.82 (d, 1H), 4.93-4.98 (m, 1H), 3.84 (s, 2H), | | |
| | 2.85-3.08 (m, 4H), 2.65-2.80 (m, 1H), 2.15-2.29 (m, 4H), 1.71-1.84 (m, 4H), 1.49 (d, | | |
| | 3H) | | |
| 26.23 | 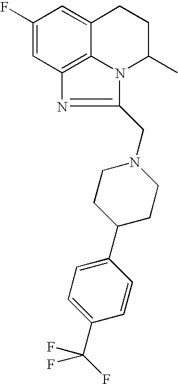 | 8-fluoro-4-methyl-2-[4- (4- trifluoromethylphenyl)- piperidin-1-ylmethyl]-5,6- dihydro-4H- imidazo[4,5,1-ij]quinoline | 54 mg (70%), brown solid |
| NMR | 7.55-7.57 (m, 2H), 7.22-7.35 (m, 3H), 6.81-6.85 (d, 1H), 4.95-4.97 (m, 1H), 3.85 (s, | | |
| | 2H), 2.88-3.11 (m, 4H), 2.58-2.70 (m, 1H), 2.06-2.36 (m, 4H), 1.72-1.85 (m, 4H), 1.49 | | |
| | (d, 3H) | | |
| 26.24 | 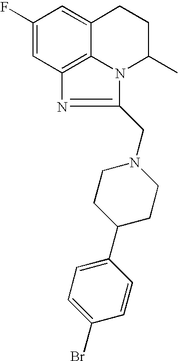 | 8-fluoro-4-methyl-2-[4- (4-bromo-phenyl)- piperidin-1-ylmethyl]-5,6- dihydro-4H- imidazo[4,5,1-ij]quinoline | 36 mg (46%), brown solid |
| NMR | 7.40-7.46 (m, 2H), 7.22 (dxd, 3H), 7.09-7.13 (m, 2H), 6.82 (d, 1H), 4.93-4.98 (m, 1H), | | |
| | 3.84 (s, 2H), 2.82-3.08 (m, 4H), 2.42-2.60 (m, 1H), 2.07-2.30 (m, 4H), 1.71-1.86 (m, | | |
| | 4H), 1.49 (d, 3H) | | |
| 26.25 | 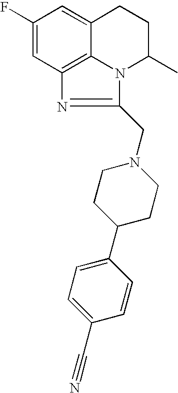 | 8-fluoro-4-methyl-2-[4- (4-cyano-phenyl)- piperidin-1-ylmethyl]-5,6- dihydro-4H- imidazo[4,5,1-ij]quinoline | 56.2 mg (81%), brown solid |
| NMR | 7.57-7.60 (m, 2H), 7.31-7.34 (m, 2H), 7.22 (dxd, 1H), 6.82 (d, 1H), 4.91-4.97 (m, 1H), | | |
| | 3.84 (s, 2H), 2.82-3.10 (m, 4H), 2.48-2.65 (m, 1H), 2.15-2.31 (m, 4H), 1.73-1.83 (m, | | |
| | 4H), 1.48 (d, 3H) | | |
| 26.26 | 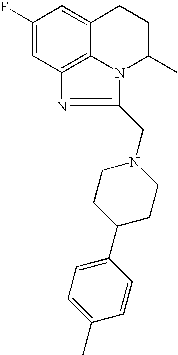 | 8-fluoro-4-methyl-2-[4- (4-methylphenyl)- piperidin-1-ylmethyl]-5,6- dihydro-4H- imidazo[4,5,1-ij]quinoline | 59.6 mg (89%), brown solid |
| NMR | 7.22-7.26 (m, 1H), 7.13 (s, 4H), 7.22 (dxd, 1H), 6.82 (d, 1H), 4.94-5.00 (m, 1H), 3.85 | | |
| | (s, 2H), 2.86-3.08 (m, 4H), 2.48-2.65 (m, 1H), 2.33 (s, 3H), 2.14-2.30 (m, 4H), 1.74-1.83 | | |
| | (m, 4H), 1.49 (d, 3H) | | |
| 26.27 | 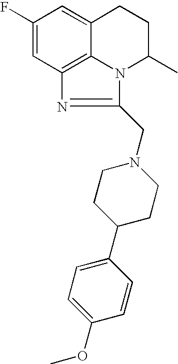 | 8-fluoro-4-methyl-2-[4- (4-methoxyphenyl)- piperidin-1-ylmethyl]-5,6- dihydro-4H- imidazo[4,5,1-ij]quinoline | 70.3 mg (99%), brown solid |
| NMR | 7.24 (dxd, 1H), 7.14-7.21 (m, 2H), 6.80-6.87 (m, 3H), 4.94-5.00 (m, 1H), 3.84 (s, 2H), | | |
| | 3.80 (s, 3H), 2.86-3.08 (m, 4H), 2.42-2.58 (m, 1H), 2.15-2.30 (m, 4H), 1.71-1.82 (m, | | |
| | 4H), 1.49 (d, 3H) | | |
| 26.28 | 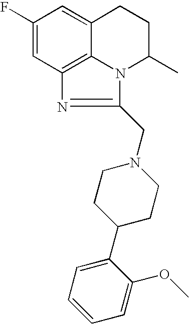 | 8-fluoro-4-methyl-2-[4- (2-methoxyphenyl)- piperidin-1-ylmethyl]-5,6- dihydro-4H- imidazo[4,5,1-ij]quinoline | 53.5 mg (76%), brown solid |
| NMR | 7.17-7.26 (m, 3H), 6.83-6.93 (m, 3H), 4.98-5.00 (m, 1H), 3.85 (s, 2H), 3.84 (s, 3H), | | |
| | 2.80-3.06 (m, 5H), 2.15-2.34 (m, 4H), 1.71-1.82 (m, 4H), 1.50 (d, 3H) | | |
| 26.29 | 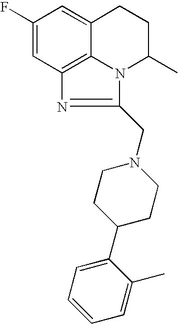 | 8-fluoro-4-methyl-2-[4- (2-methylphenyl)- piperidin-1-ylmethyl]-5,6- dihydro-4H- imidazo[4,5,1-ij]quinoline | 63.7 mg (95%), brown solid |
| NMR | 7.11-7.26 (m, 5H), (dxd, 1H), 6.82 (d, 1H), 4.96-5.01 (m, 1H), 3.87 (s, 2H), 2.93-3.11 | | |
| | (m, 4H), 2.65-2.82 (m, 1H), 2.15-2.36 (m, 7H), 1.75-1.82 (m, 4H), 1.51 (d, 3H) | | |
| 26.30 | 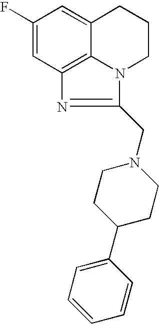 | 8-fluoro-2-[(4- phenylpiperidin-1- yl)methyl]-5,6-dihydro- 4H-imidazo]4,5,1- ij]quinoline | 43.1 mg (92%), brown solid |
| NMR | 7.21-7.34 (m, 6H), 6.78-6.82 (m, 1H), 4.31-4.35 (m, 2H), 3.85 (s, 2H), 2.97-3.02 (m, | | |
| | 4H), 1.65-1.85 (m, 1H), 2.23-2.32 (m, 2H), 1.73-1.88 (m, 4H) | | |
| 26.31 | 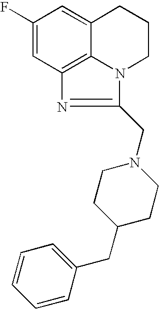 | 8-fluoro-2-[(4- benzylpiperidin-1- yl)methyl]-5,6-dihydro- 4H-imidazo[4,5,1- ij]quinoline | 45.1 mg (93%), brown oil |
| NMR | 7.13-7.31 (m, 6H), 6.79 (dxd, 1H), 4.27-4.30 (m, 2H), 3.77 (s, 2H), 2.97 (t, 2H), 2.82-2.86 | | |
| | (m, 2H), 2.53 (d, 2H), 2.20-2.54 (m, 2H), 2.03-2.12 (m, 2H), 1.48-1.66 (m, 3H), | | |
| | 1.26-1.30 (m, 2H) | | |
| 26.32 | 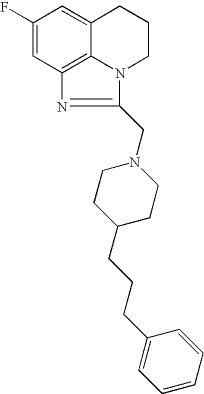 | 8-fluoro-2-[4-(3-phenyl propyl)piperidin-1- ylmethyl]-5,6-dihydro- 4H-imidazo[4,5,1- ij]quinoline | 56.3 mg (100%), brown oil |
| NMR | 7.17-7.32 (m, 6H), 6.80 (dxd, 1H), 4.24-4.30 (m, 2H), 3.78 (s, 2H), 2.96 (t, 2H), 2.82-2.86 | | |
| | (m, 2H), 2.60 (t, 2H), 2.19-2.24 (m, 2H), 2.06-2.13 (m, 2H), 1.61-1.69 (m, 4H), | | |
| | 1.19-1.30 (m, 5H) | | |
| 26.33 | 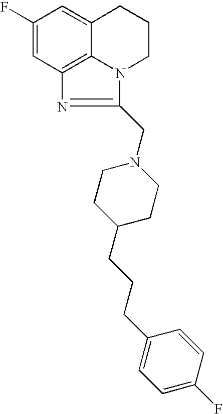 | 8-fluoro-2-{4-[3-(4- fluorophenyl) propyl]piperidin-1- ylmethyl}-5,6-dihydro- 4H-imidazo[4,5,1- ij]quinoline | 51.6 mg (94%), brown oil |
| NMR | 7.51 (s, 1H), 7.10-7.14 (m, 2H), 6.93-6.99 (m, 2H), 6.78 (dxd, 1H), 4.25-4.29 (m, 2H), | | |
| | 3.77 (s, 2H), 2.95 (t, 2H), 2.80-2.84 (m, 2H), 2.56 (t, 2H), 2.19-2.23 (m, 2H), 2.05-2.13 | | |
| | (m, 2H), 1.57-1.68 (m, 4H), 1.18-1.28 (m, 5H) | | |
| 26.34 | 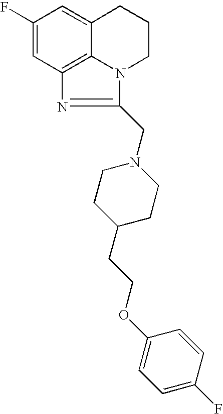 | 8-fluoro-2-{4-[2-(4- fluorophenoxy) ethyl]piperidin-1- ylmethyl}-5,6-dihydro- 4H-imidazo[4,5,1- ij]quinoline | 49 mg (89%), brown oil |
| NMR | 7.20 (dxd, 1H), 6.94-7.00 (m, 2H), 6.77-6.84 (m, 3H), 6.78 (dxd, 1H), 4.27-4.30 (m, | | |
| | 2H), 3.95 (t, 2H), 3.78 (s, 2H), 2.96 (t, 2H), 2.85-2.88 (m, 2H), 2.06-2.25 (m, 4H), | | |
| | 2.05-1.68-1.74 (m, 4H), 1.48-1.62 (m, 1H), 1.25-1.31 (m, 2H) | | |
| 26.35 | 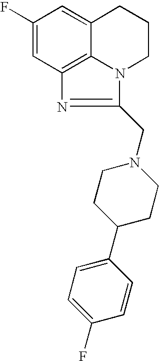 | 8-fluoro-2-{[4-(4- fluorophenyl)piperidin-1- yl]methyl}-5,6-dihydro- 4H-imidazo[4,5,1- ij]quinoline | 51 mg, (104%), brown oil |
| NMR | 7.16-7.28 (m, 3H), 6.96-7.02 (m, 2H), 6.78 (dxd, 1H), 4.30-4.34 (m, 2H), 3.85 (s, 2H), | | |
| | 2.97-3.00 (m, 4H), 2.45-2.60 (m, 1H), 2.19-2.31 (m, 4H), 1.68-1.86 (m, 4H) | | |
| 26.36 | 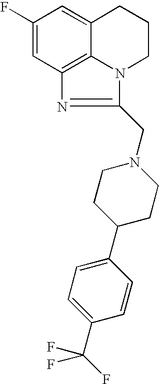 | 8-fluoro-2-{[4-(4- trifluoromethyl- phenyl)piperidin-1- yl]methyl}-5,6-dihydro- 4H-imidazo[4,5,1- ij]quinoline | 49.8 mg (89%), brown oil |
| NMR | 7.56 (d, 2H), 7.32-7.35 (m, 2H), 7.21 (dxd, 1H), 6.79 (dxd, 1H), 4.30-4.34 (m, 2H), | | |
| | 3.85 (s, 2H), 2.97-3.04 (m, 4H), 2.450-2.65 (m, 1H), 2.23-2.33 (m, 4H), 1.73-1.87 (m, | | |
| | 4H) | | |
| 26.37 | 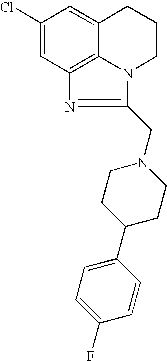 | 8-chloro-2-{[4-(4- fluorophenyl)piperidin-1- yl]methyl}-5,6-dihydro- 4H-imidazo[4,5,1- ij]quinoline | 53 mg, (106%), off white solid |
| NMR | 7.53 (s, 1H), 7.15-7.20 (m, 2H), 6.95-7.01 (m, 3H), 4.30-4.33 (m, 2H), 3.84 (s, 2H), | | |
| | 2.95-3.00 (m, 4H), 2.50-2.56 (m, 1H), 2.22-2.30 (m, 4H), 1.68-1.85 (m, 4H) | | |
| 26.38 | 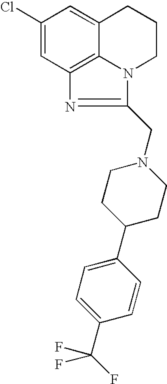 | 8-chloro-2-{[4-(4- trifluoromethyl- phenyl)piperidin-1- yl]methyl}-5,6-dihydro- 4H-imidazo[4,5,1- ij]quinoline | 50.2 mg, (93%), off white solid |
| NMR | 7.53-7.57 (m, 3H), 7.32-7.35 (m, 2H), 7.00 (s, 1H), 4.30-4.33 (m, 2H), 3.85 (s, 2H), | | |
| | 2.97-3.03 (m, 4H), 2.55-2.65 (m, 1H), 2.18-2.33 (m, 4H), 1.74-1.87 (m, 4H) | | |
| 26.39 | 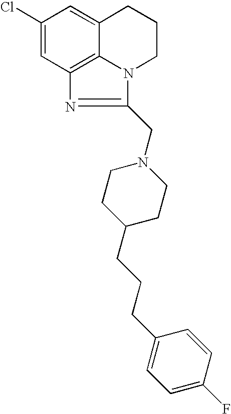 | 8-chloro-2-{4-[3-(4- fluorophenyl) propyl]piperidin-1- ylmethyl}-5,6-dihydro- 4H-imidazo[4,5,1- ij]quinoline | 50.2 mg (100%), brown oil |
| NMR | 7.20 (dxd, 1H), 7.10-7.14 (m, 2H), 6.93-6.99 (m, 2H), 6.78 (dxd, 1H), 4.26-4.30 (m, | | |
| | 2H), 3.77 (s, 2H), 2.96 (t, 2H), 2.81-2.85 (m, 2H), 2.56 (t, 2H), 2.20-2.24 (m, 2H), | | |
| | 2.05-2.12 (m, 2H), 1.57-1.68 (m, 4H), 1.18-1.28 (m, 5H) | | |
| 26.40 | 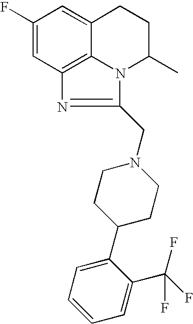 | 8-fluoro-4-methyl-2-[4- (2- trifluoromethylphenyl)- piperidin-1-ylmethyl]-5,6- dihydro-4H- imidazo[4,5,1-ij]quinoline | 29.6 mg (39%), brown solid |
| NMR | 7.60-7.62 (m, 1H), 7.42-7.47 (m, 3H), 7.20-7.30 (m, 2H), 6.83 (d, 1H), 4.95-4.97 (m, | | |
| | 1H), 3.85 (s, 2H), 2.88-3.06 (m, 4H), 2.17-2.40 (m, 8H), 1.79-1.82 (m, 4H), 1.51 (d, | | |
| | 3H) | | |
| 26.41 | 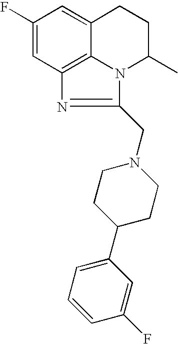 | 8-fluoro-4-methyl-2-[4- (3-fluoro-phenyl)- piperidin-1-ylmethyl]-5,6- dihydro-4H- imidazo[4,5,1-ij]quinoline | 48.3 mg (71%), brown solid |
| NMR | 7.22-7.30 (m, 2H), 6.81-7.02 (m, 4H), 4.94-4.99 (m, 1H), 3.85 (s, 2H), 2.85-3.09 (m, | | |
| | 4H), 2.50-2.60 (m, 1H), 2.16-2.31 (m, 4H), 1.73-1.86 (m, 4H), 1.50 (d, 3H) | | |
| 26.42 | 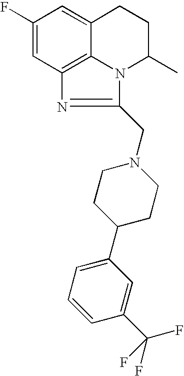 | 8-fluoro-4-methyl-2-[4- (3- trifluoromethylphenyl)- piperidin-1-ylmethyl]-5,6- dihydro-4H- imidazo[4,5,1-ij]quinoline | 54.5 mg (71%), brown solid |
| NMR | 7.41-7.48 (m, 4H), 7.22-7.26 (m, 1H), 6.83 (d, 1H), 4.95-4.99 (m, 1H), 3.86 (s, 2H), | | |
| | 2.88-3.12 (m, 4H), 2.58-2.64 (m, 1H), 2.17-2.32 (m, 4H), 1.51-1.87 (m, 4H), 1.50 (d, | | |
| | 3H) | | |
| 26.43 | 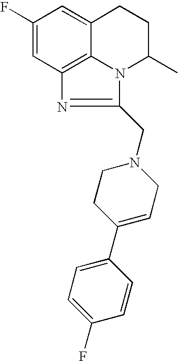 | 8-fluoro-4-methyl-2-{[4- (4-fluorophenyl)-3,6- dihydropyridin-1(2H)- yl]methyl}-5,6-dihydro- 4H-imidazo[4,5,1- ij]quinoline | 27 mg, (71%) yellow oil |
| NMR | 7.33-7.38 (m, 2H), 7.26 (dxd, 1H), 6.98-7.04 (m, 2H), 6.81-6.85 (m, 1H), 6.01-6.03 | | |
| | (m, 1H), 4.96-4.98 (m, 1H), 3.95 (d, 2H), 3.26-3.27 (m, 2H), 3.05-3.15 (m, 1H), 2.81-2.93 | | |
| | (m, 3H), 2.53-2.55 (m, 2H), 2.12-2.20 (m, 2H), 1.27 (d, 3H) | | |
| 26.44 | 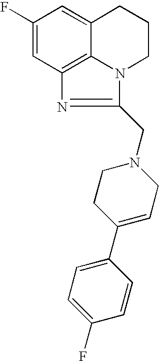 | 8-fluoro-2-{[4-(4- fluorophenyl)-3,6- dihydropyridin-1(2H)- yl]methyl}-5,6-dihydro- 4H-imidazo[4,5,1- ij]quinoline | 21.7 mg (59%), yellow oil |
| NMR | 7.33-7.38 (m, 2H), 7.25 (dxd, 1H), 6.99-7.04 (m, 2H), 6.80-6.83 (m, 1H), 6.01-6.03 | | |
| | (m, 1H), 4.30-4.34 (m, 2H), 3.95 (s, 2H), 3.25-3.26 (m, 2H), 2.98 (t, 2H), 2.80 (t, 3H), | | |
| | 2.52-2.53 (m, 2H), 2.20-2.28 (m, 2H) | | |
| 26.45 | 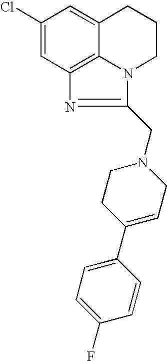 | 8-chloro-2-{[4-(4- fluorophenyl)-3,6- dihydropyridin-1(2H)- yl]methyl}-5,6-dihydro- 4H-imidazo[4,5,1- ij]quinoline | 39.6 mg (100%), yellow oil |
| NMR | 7.54 (s, 1H), 7.32-7.37 (m, 2H), 6.98-7.04 (m, 3H), 6.80-6.83 (m, 1H), 6.00-6.02 (m, | | |
| | 1H), 4.29-4.33 (m, 2H), 3.94 (s, 2H), 3.24-3.26 (m, 2H), 2.96 (t, 2H), 2.79 (t, 3H), | | |
| | 2.52-2.53 (m, 2H), 2.19-2.27 (m, 2H) | | |
| 26.46 | 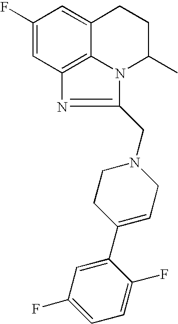 | 8-fluoro-4-methyl-2-{[4- (2,5-difluorophenyl)-3,6- dihydropyridin-1(2H)- yl]methyl}-5,6-dihydro- 4H-imidazo[4,5,1- ij]quinoline | 42.5 mg, (100%) yellow oil |
| NMR | 7.21-7.26 (m, 2H), 6.79-6.86 (m, 3H), 6.81-6.85 (m, 1H), 5.91-5.93 (m, 1H), 4.96-4.99 | | |
| | (m, 1H), 3.95 (d, 2H), 3.25-3.27 (m, 2H), 3.00-3.13 (m, 1H), 2.82-2.95 (m, 3H), 2.53-2.55 | | |
| | (m, 2H), 2.14-2.20 (m, 2H), 1.47 (d, 3H) | | |
| 26.47 | 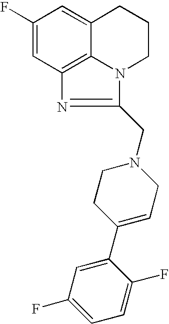 | 8-fluoro-2-{[4-(2,5- difluorophenyl)-3,6- dihydropyridin-1(2H)- yl]methyl}-5,6-dihydro- 4H-imidazo[4,5,1- ij]quinoline | 29.2 mg (81%), yellow oil |
| NMR | 7.18-7.25 (m, 2H), 6.75-6.86 (m, 3H), 6.80-6.83 (m, 1H), 5.91-5.92 (m, 1H), 4.30-4.34 | | |
| | (m, 2H), 3.94 (s, 2H), 3.24-3.27 (m, 2H), 2.98 (t, 2H), 2.78 (t, 3H), 2.51-2.52 (m, 2H), | | |
| | 2.24-2.28 (m, 2H) | | |
| 26.48 | 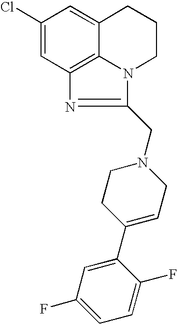 | 8-chloro-2-{[4-(2,5- difluorophenyl)-3,6- dihydropyridin-1(2H)- yl]methyl}-5,6-dihydro- 4H-imidazo[4,5,1- ij]quinoline | 32.9 mg (88%), yellow oil |
| NMR | 7.53 (s, 2H), 7.21-7.28 (m, 1H), 7.00 (s, 1H), 6.78-6.82 (m, 2H), 5.91-5.92 (m, 1H), | | |
| | 4.30-4.33 (m, 2H), 3.94 (s, 2H), 3.23-3.26 (m, 2H), 2.96 (t, 2H), 2.77 (t, 3H), 2.51-2.52 | | |
| | (m, 2H), 2.22-2.26 (m, 2H) | | |
| 26.49 | 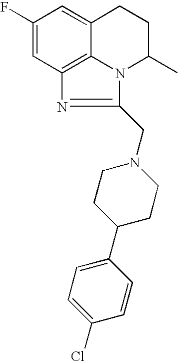 | 8-fluoro-4-methyl-2-[4- (4-chlorophenyl)- piperidin-1-ylmethyl]-5,6- dihydro-4H- imidazo[4,5,1-ij]quinoline | 33.5 mg (47%), brown solid |
| NMR | 7.14-7.28 (m, 5H), 6.82 (d, 1H), 4.94-4.97 (m, 1H), 3.84 (s, 2H), 2.88-3.08 (m, 4H), | | |
| | 2.48-2.60 (m, 1H), 2.15-2.33 (m, 4H), 1.67-1.87 (m, 4H), 1.49 (d, 3H) | | |
| 26.50 | 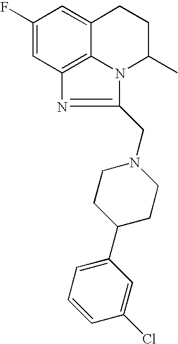 | 8-fluoro-4-methyl-2-[4- (3-chlorophenyl)- piperidin-1-ylmethyl]-5,6- dihydro-4H- imidazo[4,5,1-ij]quinoline | 13 mg (18%), brown solid |
| NMR | 7.10-7.28 (m, 5H), 6.82 (d, 1H), 4.94-4.97 (m, 1H), 3.85 (s, 2H), 2.94-3.09 (m, 4H), | | |
| | 2.42-2.58 (m, 1H), 2.16-2.30 (m, 4H), 1.72-1.86 (m, 4H), 1.50 (d, 3H) | | |
| 26.51 | 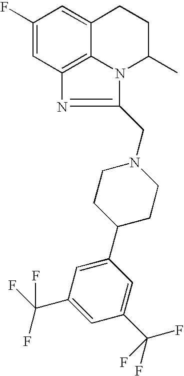 | 8-fluoro-4-methyl-2-[4- (3,5-bis(trifluoromethyl)- phenyl)-piperidin-1- ylmethyl]-5,6-dihydro- 4H-imidazo[4,5,1- ij]quinoline | 69.6 mg (78%), brown solid |
| NMR | 7.67-7.73 (m, 3H), 7.24 (dxd, 1H), 6.82 (d, 1H), 4.93-4.98 (m, 1H), 3.86 (s, 2H), 2.95-3.11 | | |
| | (m, 4H), 2.62-2.78 (m, 1H), 2.17-2.34 (m, 4H), 1.77-1.91 (m, 4H), 1.50 (d, 3H) | | |
| 26.52 | 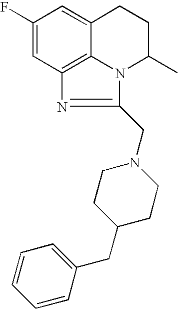 | 8-fluoro-4-methyl-2-[4- benzylphenyl)-piperidin- 1-ylmethyl]-5,6-dihydro- 4H-imidazo[4,5,1- ij]quinoline | 54 mg (80%), brown solid |
| NMR | 7.13-7.31 (m, 6H), 6.81 (d, 1H), 4.92-4.96 (m, 1H), 3.77 (s, 2H), 3.02-3.18 (m, 2H), | | |
| | 2.86-2.93 (m, 2H), 2.42-2.58 (m, 1H), 2.54 (d, 2H), 2.07-2.19 (m, 4H), 1.60-1.67 (m, | | |
| | 4H), 1.47 (d, 3H), 1.27-1.38 (m, 2H) | | |
| 26.53 | 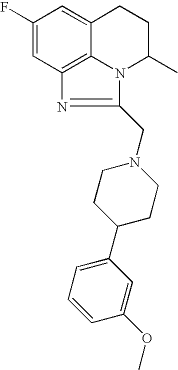 | 8-fluoro-4-methyl-2-[4- (3-methoxyphenyl)- piperidin-1-ylmethyl]-5,6- dihydro-4H- imidazo[4,5,1-ij]quinoline | 60.3 mg (86%), brown solid |
| NMR | 7.21-7.28 (m, 2H), 6.75-6.85 (m, 4H), 4.96-4.98 (m, 1H), 3.85 (s, 2H), 3.82 (s, 3H), | | |
| | 2.94-3.08 (m, 4H), 2.50-2.58 (m, 1H), 2.16-2.30 (m, 4H), 1.75-1.85 (m, 4H), 1.50 (d, | | |
| | 3H) | | |
| 26.54 | 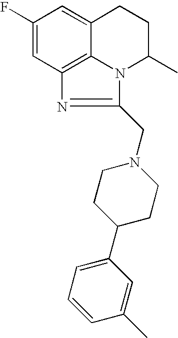 | 8-fluoro-4-methyl-2-[4- (3-methylphenyl)- piperidin-1-ylmethyl]-5,6- dihydro-4H- imidazo[4,5,1-ij]quinoline | 57.4 mg (85%), brown solid |
| NMR | 7.19-7.28 (m, 2H), 7.02-7.05 (m, 3H), 6.82 (d, 1H), 4.96-5.00 (m, 1H), 3.85 (s, 2H), | | |
| | 2.94-3.11 (m, 4H), 2.50-2.58 (m, 1H), 2.35 (s, 3H), 2.16-2.31 (m, 4H), 1.75-1.86 (m, | | |
| | 4H), 1.50 (d, 3H) | | |
| 26.55 | 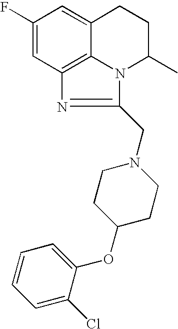 | 8-fluoro-4-methyl-2-[4- (2-chlorophenoxy)- piperidin-1-ylmethyl]-5,6- dihydro-4H- imidazo[4,5,1-ij]quinoline | 47.5 mg (64%). |
| NMR | 7.39 (dxd, 1H), 7.14-7.20 (m, 3H), 6.82-6.99 (m, 1H), 6.82 (d, 1H), 4.92-4.96 (m, 1H), | | |
| | 4.38-4.40 (m, 1H), 3.84 (s, 2H), 3.05-3.18 (m, 2H), 2.86-2.97 (m, 3H), 2.40-2.48 (m, | | |
| | 1H), 2.16-2.19 (m, 2H), 1.47-1.99 (m, 4H), 1.48 (d, 3H) | | |
| 26.56 | 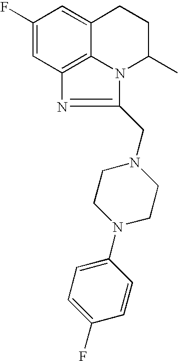 | 8-fluoro-4-methyl-2- {[4(4- fluorophenyl)piperizin-1- yl]methyl}-5,6-dihydro- 4H-imidazo[4,5,1- ij]quinoline | 51.2 mg (100%), brown foam |
| NMR | 7.25 (dxd, 1H), 6.81-6.99 (m, 5H), 4.90-4.95 (m, 1H), 3.88 (s, 2H), 2.87-3.14 (m, 6H), | | |
| | 2.72-2.75 (m, 4H), 2.12-2.18 (m, 2H), 1.48 (d, 3H) | | |
| 26.57 | 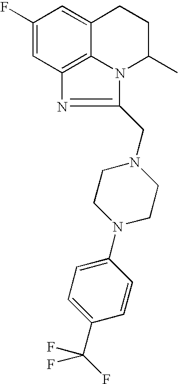 | 8-fluoro-4-methyl-2- {[4(4-trifluoromethyl- phenyl)piperizin-1- yl]methyl}-5,6-dihydro- 4H-imidazo[4,5,1- ij]quinoline | 52.2 mg (96.1%), brown foam |
| NMR | 7.48 (d, 2H), 7.24 (dxd, 1H), 6.92 (d, 2H), 6.82-6.86 (m, 1H), 4.90-4.94 (m, 1H), 3.88 | | |
| | (s, 2H), 3.27-3.30 (m, 4H), 2.88-3.14 (m, 2H), 2.72-2.75 (m, 4H), 2.13-2.19 (m, 2H), | | |
| | 1.49 (d, 3H) | | |
| 26.58 | 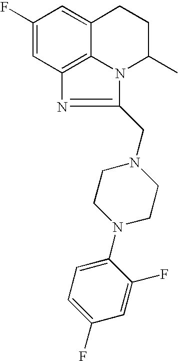 | 8-fluoro-4-methyl-2- {[4(2,4- difluorophenyl)piperizin- 1-yl]methyl}-5,6-dihydro- 4H-imidazo[4,5,1- ij]quinoline | 53.2 mg (100%), brown solid |
| NMR | 7.24 (dxd, 1H), 6.78-6.90 (m, 4H), 4.90-4.95 (m, 1H), 3.88 (s, 2H), 3.02-3.07 (m, 5H), | | |
| | 2.88-3.10 (m, 2H), 2.73-2.77 (m, 4H), 2.13-2.19 (m, 2H), 1.49 (d, 3H) | | |
| 26.59 | 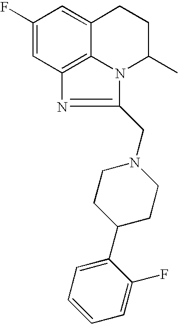 | 8-fluoro-4-methyl-2-[4- (2-fluoro-phenyl)- piperidin-1-ylmethyl]-5,6- dihydro-4H- imidazo[4,5,1-ij]quinoline | 128.9 mg brown solid |
| NMR | 7.02-7.26 (m, 4H), 6.84 (d, 1H), 4.90-4.95 (m, 1H), 3.86 (s, 2H), 2.82-3.10 (m, 5H), | | |
| | 2.17-2.35 (m, 4H), 1.79-1.86 (m, 4H), 1.50 (d, 3H) | | |
| 26.60 | 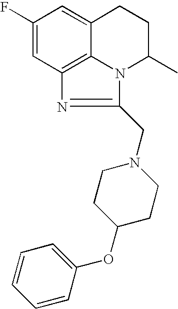 | 8-fluoro-4-methyl-2-[4- phenoxy-piperidin-1- ylmethyl]-5,6-dihydro- 4H-imidazo[4,5,1- ij]quinoline | 27.5 mg (40.7%), brown solid. |
| NMR | 7.21-7.32 (m, 3H), 7.14-6.90-6.99 (m, 3H), 6.82 (d, 1H), 4.94-4.96 (m, 1H), 4.33-4.36 | | |
| | (m, 1H), 3.84 (s, 2H), 3.05-3.18 (m, 1H), 2.84-2.93 (m, 3H), 2.40-2.48 (m, 2H), 2.14-2.19 | | |
| | (m, 2H), 1.73-1.83 (m, 4H), 1.48 (d, 3H) | | |
| 26.61 | 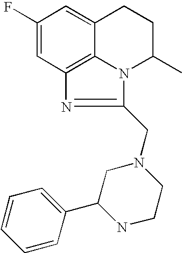 | 8-fluoro-4-methyl-2-[3- phenyl-piperazin-1- ylmethyl]-5,6-dihydro- 4H-imidazo[4,5,1- ij]quinoline | 104.51 mg |
| NMR | 7.19-7.39 (m, 6H), 6.82 (d, 2H), 4.93-4.96 (m, 1H), 3.84-3.90 (m, 4H), 2.86-3.11 (m, | | |
| | 6H), 2.06-2.32 (m, 6H), 1.48-1.51 (m, 3H) | | |
| 26.62 | 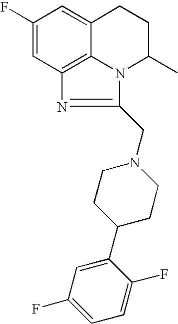 | 8-fluoro-4-methyl-2-{[4- (2,5- difluorophenyl)piperidin- 1-yl]methyl}-5,6-dihydro- 4H-imidazo[4,5,1- ij]quinoline | 26.6 mg (37.2%), brown oil |
| NMR | 7.17-7.28 (m, 2H), 6.75-6.85 (m, 3H), 4.94-4.96 (m, 1H), 3.85 (s, 2H), 2.80-3.08 (m, | | |
| | 5H), 2.25-2.35 (m, 2H), 2.16-2.20 (m, 2H), 1.75-1.83 (m, 4H), 1.49 (d, 3H) | | |
| 26.63 | 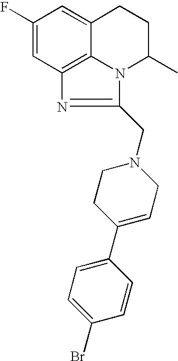 | 8-fluoro-4-methyl-2-{[4- (4-bromophenyl)-3,6- dihydropyridin-1(2H)- yl]methyl}-5,6-dihydro- 4H-imidazo[4,5,1- ij]quinoline | 31.7 mg, (72%) brown oil |
| NMR | 7.43-7.50 (m, 2H), 7.23-7.28 (m, 3H), 6.98-7.04 (m, 2H), 6.83 (m, 1H), 6.08-6.10 (m, | | |
| | 1H), 4.95-4.98 (m, 1H), 3.96 (d, 2H), 3.25-3.27 (m, 2H), 3.05-3.15 (m, 1H), 2.81-2.93 | | |
| | (m, 3H), 2.53-2.55 (m, 2H), 2.12-2.17 (m, 2H), 1.45 (d, 3H) | | |
| 26.64 | 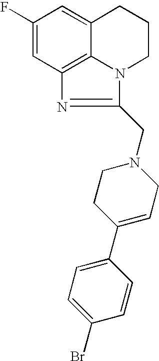 | 8-fluoro-2-{[4-(4- bromophenyl)-3,6- dihydropyridin-1(2H)- yl]methyl}-5,6-dihydro- 4H-imidazo[4,5,1- ij]quinoline | 24.2 mg (57%), brown oil |
| NMR | 7.43-7.46 (m, 2H), 7.21-7.27 (m, 3H), 6.99-7.04 (m, 2H), 6.81 (dxd, 1H), 6.08-6.09 | | |
| | (m, 1H), 4.29-4.33 (m, 2H), 3.95 (s, 2H), 3.24-3.27 (m, 2H), 2.98 (t, 2H), 2.80 (t, 3H), | | |
| | 2.52-2.53 (m, 2H), 2.22-2.26 (m, 2H) | | |
| 26.65 | 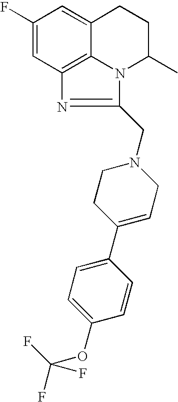 | 8-fluoro-4-methyl-2-{[4- (4- trifluoromethoxyphenyl)- 3,6-dihydropyridin- 1(2H)-yl]methyl}-5,6- dihydro-4H- imidazo[4,5,1-ij]quinoline | 41.3 mg, (93%) brown oil |
| NMR | 7.40-7.42 (m, 2H), 7.16-7.28 (m, 3H), 6.83 (dxd, 2H), 6.08-6.10 (m, 1H), 4.95-4.98 (m, | | |
| | 1H), 3.96 (d, 2H), 3.27-3.29 (m, 2H), 3.05-3.15 (m, 1H), 2.83-2.93 (m, 3H), 2.54-2.57 | | |
| | (m, 2H), 2.13-2.19 (m, 2H), 1.46 (d, 3H) | | |
| 26.66 | 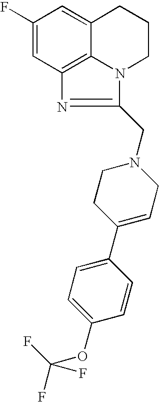 | 8-fluoro-2-{[4-(4- trifluoromethoxyphenyl)- 3,6-dihydropyridin- 1(2H)-yl]methyl}-5,6- dihydro-4H- imidazo[4,5,1-ij]quinoline | 22.3 mg (52%), brown oil |
| NMR | 7.39-7.42 (m, 2H), 7.16-7.25 (m, 3H), 6.99-7.04 (m, 2H), 6.80 (dxd, 1H), 6.06-6.09 | | |
| | (m, 1H), 4.29-4.33 (m, 2H), 3.95 (s, 2H), 3.26-3.28 (m, 2H), 2.98 (t, 2H), 2.81 (t, 3H), | | |
| | 2.54-2.55 (m, 2H), 2.21-2.29 (m, 2H) | | |
| 26.67 | 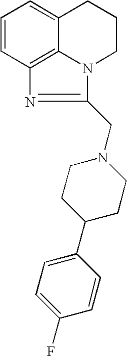 | 2-[(4-(4- fluorophenyl)piperidin-1- yl)methyl]-5,6-dihydro- 4H-imidazo[4,5,1- ij]quinoline | 58.3 mg (86%), brown solid |
| NMR | 7.56 (d, 1H), 7.15-7.21 (m, 3H), 6.96-7.03 (m, 3H), 4.32-4.35 (m, 2H), 3.87 (s, 2H), | | |
| | 3.00-3.07 (m, 4H), 2.49-2.54 (m, 1H), 2.26-2.31 (m, 1H), 1.68-1.85 (m, 4H) | | |
| 26.68 | 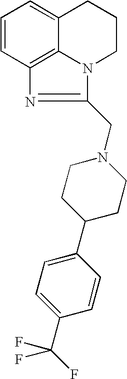 | 2-[(4-(4- trifluoromethylphenyl)- piperidin-1-yl)methyl]-5,6- dihydro-4H- imidazo[4,5,1-ij]quinoline | 42.9 mg (54%), brown solid |
| NMR | 7.55-7.58 (d, 2H), 7.32-7.35 (m, 3H), 7.13 (t, 1H), 6.74 (d, 3H), 4.52 (s, 4H), 3.91 (s, | | |
| | 2H), 3.00-3.04 (m, 2H), 2.52-2.68 (m, 1H), 2.25-2.35 (m, 2H), 1.72-1.89 (m, 4H) | | |
| 26.69 | 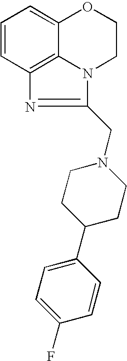 | 2-[(4-(4- fluorophenylpiperidin-1- yl)methyl]-4,5- dihydroimidazo[1,5,4- de][1,4]benzoxazine | 38.6 mg (81%), yellow solid |
| NMR | 7.55-7.58 (m, 1H), 7.15-7.21 (m, 3H), 6.96-7.03 (m, 3H), 4.32-4.37 (m, 2H), 3.84 (s, | | |
| | 2H), 2.99-3.02 (m, 4H), 2.48-2.62 (m, 1H), 2.23-2.31 (m, 2H), 1.68-1.85 (m, 4H) | | |
| 26.70 | 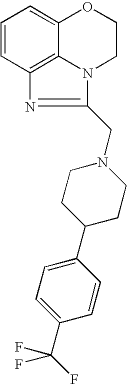 | 2-[(4-(4- trifluoromethylphenyl- piperidin-1-yl)methyl]-4,5- dihydroimidazo[1,5,4- de][1,4]benzoxazine | 66.6 mg (86%), yellow solid |
| NMR | 7.55-7.58 (m, 1H), 7.32-7.35 (m, 3H), 7.13 (t, 1H), 6.76 (d, 1H), 4.51 (s, 4H), 3.91 (s, | | |
| | 2H), 3.00-3.04 (m, 4H), 2.48-2.62 (m, 1H), 2.25-2.34 (m, 2H), 1.72-1.90 (m, 4H) | | |
| 26.71 | 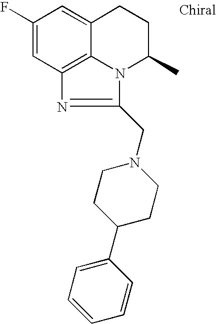 | [4R]-8-fluoro-4-methyl-2- [(4-phenylpiperidin-1- yl)methyl]-5,6-dihydro- 4H-imidazo[4,5,1- ij]quinoline | 192 mg (95%), brown foam |
| NMR | 7.18-7.34 (m, 6H), 6.81-6.84 (m, 1H), 4.95-4.99 (m, 1H), 3.85 (s, 2H) 2.93-3.10 (m, | | |
| | 4H), 2.29-2.41 (m, 1H), 2.15-2.32 (m, 4H), 1.70-1.86 (m, 3H), 1.50 (d, 3H) | | |
| 26.72 | 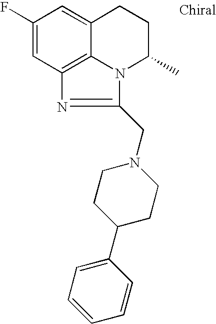 | [4S]-8-fluoro-4-methyl-2- [(4-phenylpiperidin-1- yl)methyl]-5,6-dihydro- 4H-imidazo[4,5,1- ij]quinoline | 112.2 mg (81%), brown foam |
| NMR | 7.18-7.38 (m, 6H), 6.80-6.83 (m, 1H), 4.95-4.99 (m, 1H), 3.85 (s, 2H) 2.91-3.09 (m, | | |
| | 4H), 2.29-2.41 (m, 1H), 2.14-2.31 (m, 4H), 1.65-1.84 (m, 3H), 1.49 (d, 3H), | | |
| 26.73 | 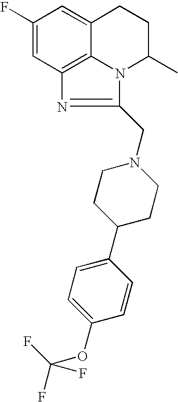 | 8-fluoro-4-methyl-2-{[4- (4- trifluoromethoxyphenyl)- piperidin-1-yl]methyl}-5,6- dihydro-4H- imidazo[4,5,1-ij]quinoline | 42.9 mg (96%), brown oil |
| NMR | 7.22-7.26 (m, 3H), 7.13-7.16 (m, 2H), 6.81-6.85 (m, 1H), 4.93-4.98 (m, 1H), 3.85 (s, | | |
| | 1H), 2.57-3.08 (m, 4H), 2.52-2.62 (m, 1H), 2.16-2.31 (m, 4H), 1.48-1.84 (m, 4H), 1.49 | | |
| | (d, 3H) | | |
| 26.74 | 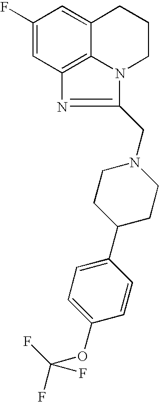 | 8-fluoro-2-{[4-(4- trifluoromethoxyphenyl)- piperidin-1-yl]methyl}-5,6- dihydro-4H- imidazo[4,5,1-ij]quinoline | 34.6 mg, (80%), brown oil |
| NMR | 7.13-7.28 (m, 5H), 6.81 (dxd, 1H), 4.30-4.34 (m, 2H), 3.85 (s, 2H), 2.97-3.02 (m, 4H), | | |
| | 2.55-2.65 (m, 1H), 2.22-2.30 (m, 4H), 1.70-1.87 (m, 4H) | | |
| 26.75 | 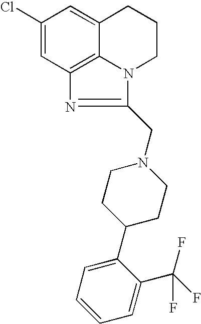 | 8-chloro-2-[(4-(2- trifluoromethylphenyl)- piperidin-1-yl)methyl]-5,6- dihydro-4H- imidazo[4,5,1-ij]quinoline | 51.9 mg (77%), brown solid |
| NMR | 7.63 (d, 1H), 7.46-7.54 (m, 3H), 7.29-32 (m, 1H), 7.02 (d, 1H), 4.31-4.35 (m, 2H), | | |
| | 3.86 (s, 2H), 2.93-3.03 (m, 5H), 2.19-2.35 (m, 4H), 1.78-1.83 (m, 4H) | | |
| 26.76 | 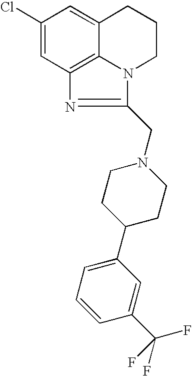 | 8-chloro-2-[(4-(3- trifluoromethylphenyl)- piperidin-1-yl)methyl]-5,6- dihydro-4H- imidazo[4,5,1-ij]quinoline | 52.2 mg (77%), brown solid |
| NMR | 7.53 (d, 1H), 7.41-7.48 (m, 4H), 7.02 (d, 1H), 4.31-4.35 (m, 2H), 3.86 (s, 2H), 2.96-3.03 | | |
| | (m, 4H), 2.52-2.68 (m, 1H), 2.25-2.33 (m, 4H), 1.73-1.89 (m, 4H) | | |
| 26.77 | 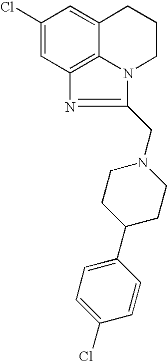 | 8-chloro-2-[(4-(4- chlorophenyl)piperidin- 1-yl)methyl]-5,6-dihydro- 4H-imidazo[4,5,1- ij]quinoline | 50.1 mg (80%), brown solid |
| NMR | 7.53 (d, 1H), 7.25-7.28 (m, 2H), 7.14-7.17 (m, 2H), 7.00 (d, 1H), 4.30-4.33 (m, 2H), | | |
| | 3.84 (s, 2H), 2.95-3.99 (m, 4H), 2.42-2.60 (m, 1H), 2.22-2.29 (m, 4H), 1.68-1.84 (m, | | |
| | 4H) | | |
| 26.78 | 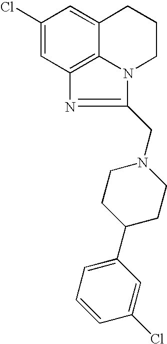 | 8-chloro-2-[(4-(3- chlorophenyl)piperidin- 1-yl)methyl]-5,6-dihydro- 4H-imidazo[4,5,1- ij]quinoline | 56.8 mg (91%), brown solid |
| NMR | 7.53 (d, 1H), 7.16-7.26 (m, 3H), 7.09-7.12 (m, 2H), 7.01 (d, 1H), 4.30-4.33 (m, 2H), | | |
| | 3.85 (s, 2H), 2.96-3.00 (m, 4H), 2.42-2.58 (m, 1H), 2.22-2.30 (m, 4H), 1.69-1.86 (m, | | |
| | 4H) | | |
| 26.79 | 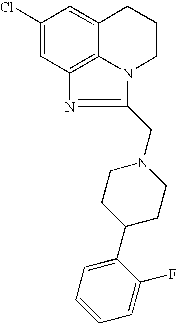 | 8-chloro-2-[(4-(2- fluorophenyl)piperidin-1- yl)methyl]-5,6-dihydro- 4H-imidazo[4,5,1- ij]quinoline | 49.5 mg (83%), brown solid |
| NMR | 7.53 (d, 1H), 7.00-7.24 (m, 5H), 4.30-4.34 (m, 2H), 3.85 (s, 2H), 2.89-3.01 (m, 5H), | | |
| | 2.21-2.34 (m, 4H), 1.75-1.84 (m, 4H) | | |
| 26.80 | 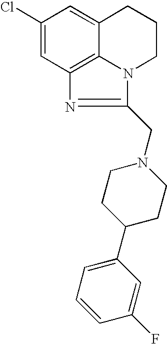 | 8-chloro-2-[(4-(3- fluorophenyl)piperidin-1- yl)methyl]-5,6-dihydro- 4H-imidazo[4,5,1- ij]quinoline | 42 mg (71%), brown solid |
| NMR | 7.53 (d, 1H), 7.24-7.27 (m, 1H), 6.87-7.01 (m, 4H), 4.30-4.34 (m, 2H), 3.84 (s, 2H), | | |
| | 2.95-3.00 (m, 4H), 2.42-2.60 (m, 1H), 2.22-2.30 (m, 4H), 1.69-1.87 (m, 4H) | | |
| 26.81 | 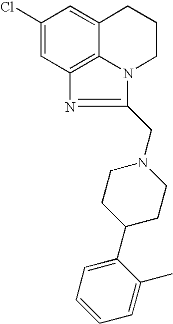 | 8-chloro-2-[(4-(2- methylphenyl)piperidin- 1-yl)methyl]-5,6-dihydro- 4H-imidazo[4,5,1- ij]quinoline | 57 mg (99%), brown solid |
| NMR | 7.54 (d, 1H), 7.11-7.28 (m, 4H), 6.87-7.01 (m, 4H), 4.32-4.36 (m, 2H), 3.87 (s, 2H), | | |
| | 2.96-3.03 (m, 4H), 2.65-2.80 (m, 1H), 2.35 (s, 3H), 2.23-2.33 (m, 4H), 1.72-1.80 (m, | | |
| | 4H) | | |
| 26.82 | 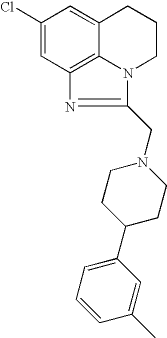 | 8-chloro-2-[(4-(3- methylphenyl)piperidin- 1-yl)methyl]-5,6-dihydro- 4H-imidazo[4,5,1- ij]quinoline | 59.6 mg (100%), brown solid |
| NMR | 7.54 (d, 1H), 7.18-7.24 (m, 1H), 7.00-7.05 (m, 4H), 4.31-4.35 (m, 2H), 3.85 (s, 2H), | | |
| | 2.95-3.00 (m, 4H), 2.42-2.58 (m, 1H), 2.35 (s, 3H), 2.22-2.31 (m, 4H), 1.72-1.83 (m, | | |
| | 4H) | | |
| 26.83 | 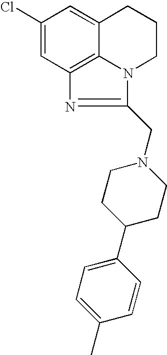 | 8-chloro-2-[(4-(4- methylphenyl)piperidin- 1-yl)methyl]-5,6-dihydro- 4H-imidazo[4,5,1- ij]quinoline | 55.8 mg (95%), brown solid |
| NMR | 7.54 (d, 1H), 7.13 (s, 4H), 7.00 (d, 1H), 4.31-4.35 (m, 2H), 3.85 (s, 2H), 2.95-2.99 (m, | | |
| | 4H), 2.42-2.58 (m, 1H), 2.34 (s, 3H), 2.22-2.31 (m, 4H), 1.75-1.82 (m, 4H) | | |
| 26.84 | 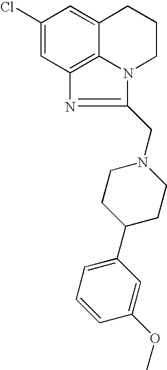 | 8-chloro-2-[(4-(3- methoxyphenyl)piperidin- 1-yl)methyl]-5,6- dihydro-4H- imidazo[4,5,1-ij]quinoline | 62 mg (100%), brown solid |
| NMR | 7.53 (d, 1H), 7.23 (t, 1H), 7.00 (d, 1H), 6.74-6.84 (m, 3H), 4.30-4.34 (m, 2H), 3.85 (s, | | |
| | 2H), 3.81 (s, 3H), 2.95-3.00 (m, 4H), 2.42-2.58 (m, 1H), 2.22-2.30 (m, 4H), 1.72-1.87 | | |
| | (m, 4H) | | |
| 26.85 | 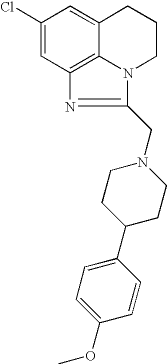 | 8-chloro-2-[(4-(4- methoxyphenyl)piperidin- 1-yl)methyl]-5,6- dihydro-4H- imidazo[4,5,1-ij]quinoline | 55 mg (90%), brown solid |
| NMR | 7.53 (d, 1H), 7.14-7.17 (m, 2H), 7.00 (d, 1H), 6.84-6.87 (m, 2H), 4.30-4.34 (m, 2H), | | |
| | 3.84 (s, 2H), 3.80 (s, 3H), 2.95-2.99 | | |
| | (m, 4H), 2.42-2.58 (m, 1H), 2.21-2.29 (m, 4H), 1.72-1.81 (m, 4H) | | |
| 26.86 | 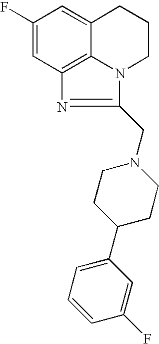 | 8-fluoro-2-{[4-(3- fluorophenyl)piperidin-1- yl]methyl}-5,6-dihydro- 4H-imidazo[4,5,1- ij]quinoline | 43 mg, (65%), brown oil |
| NMR | 7.17-7.24 (m, 3H), 7.09-7.12 (m, 2H), 6.80 (dxd, 1H), 4.30-4.34 (m, 2H), 3.85 (s, 2H), | | |
| | 2.97-3.01 (m, 4H), 2.45-2.60 (m, 1H), 2.22-2.30 (m, 4H), 1.69-1.86 (m, 4H) | | |
| 26.87 | 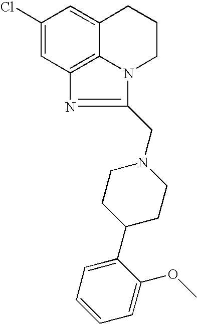 | 8-chloro-2-[(4-(2- methoxyphenyl)piperidin- 1-yl)methyl]-5,6- dihydro-4H- imidazo[4,5,1-ij]quinoline | 32.4 mg (53%), brown solid |
| NMR | 7.53 (d, 1H), 7.17-7.22 (m, 2H), 6.85-7.01 (m, 3H), 4.36-4.36 (m, 2H), 3.85 (s, 2H), | | |
| | 3.83 (s, 3H), 2.96-2.99 (m, 5H), 2.24-2.34 (m, 4H), 1.67-1.83 (m, 4H) | | |
| 26.88 | 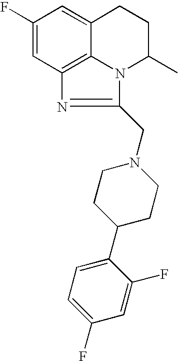 | 8-fluoro-4-methyl-2-[4- (2,4-difluoro-phenyl)- piperidin-1-ylmethyl]-5,6- dihydro-4H- imidazo[4,5,1-ij]quinoline | 11.7 mg (21.6%), brown solid |
| NMR | 7.17-7.28 (m, 2H), 6.74-6.85 (m, 3H), 4.94-4.99 (m, 1H), 3.85 (s, 2H), 2.80-3.10 (m, | | |
| | 5H), 2.15-2.33 (m, 4H), 1.65-1.80 (m, 3H), 1.75-1.80 (m, 2H), 1.49 (d, 3H), 1.27-1.30 | | |
| | (m, 1H). | | |
| 26.89 | 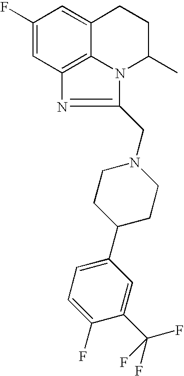 | 8-fluoro-4-methyl-2-[4- (4-fluoro-3- trifluoromethyl-phenyl)- piperidin-1-ylmethyl]-5,6- dihydro-4H- imidazo[4,5,1-ij]quinoline | 38.4 mg (54%), brown gum |
| NMR | 7.38-7.44 (m, 2H), 7.22-7.25 (m, 1H), 7.10-7.16 (m, 1H), 6.81-6.85 (m, 1H), 4.96-4.99 | | |
| | (m, 1H), 3.85 (s, 2H), 2.80-3.11 (m, 4H), 2.55-2.65 (m, 1H), 2.15-2.40 (m, 5H), 1.70-1.92 | | |
| | (m, 3H), 1.49 (d, 3H). | | |
| 26.90 | 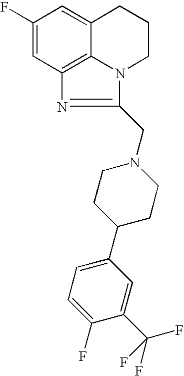 | 8-fluoro-2-[4-(4-fluoro-3- trifluoromethyl-phenyl)- piperidin-1-ylmethyl]-5,6- dihydro-4H- imidazo[4,5,1-ij]quinoline | 39 mg, (56%), brown gum |
| NMR | 7.38-7.45 (m, 2H), 7.09-7.25 (m, 2H), 6.78-6.82 (m, 1H), 4.28-4.33 (m, 2H), 3.85 (s, | | |
| | 2H), 2.80-3.11 (m, 4H), 2.52-2.65 (m, 1H), 2.19-2.30 (m, 4H), 1.60-1.85 (m, 4H). | | |
| 26.91 | 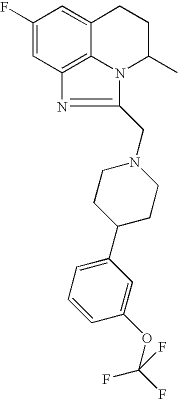 | 8-fluoro-4-methyl-2-{[4- (3- trifluoromethoxyphenyl)- piperidin-1-yl]methyl}-5,6- dihydro-4H- imidazo[4,5,1-ij]quinoline | 21.2 mg (55%), brown gum |
| NMR | 7.30-7.35 (m, 1H), 7.15-7.26 (m, 2H), 7.05-7.08 (m, 2H), 6.82-6.85 (m, 1H), 4.95-4.97 | | |
| | (m, 1H), 3.85 (s, 1H), 2.77-3.09 (m, 4H), 2.52-2.62 (m, 1H), 2.18-2.40 (m, 4H), 1.65-1.85 | | |
| | (m, 4H), 1.50 (d, 3H) | | |
| 26.92 | 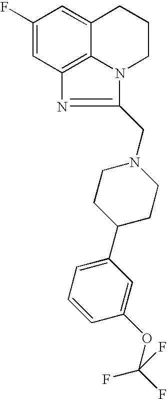 | 8-fluoro-2{[4-(3- trifluoromethoxy phenyl) piperidin-1-yl]methyl}- 5,6-dihydro-4H- imidazo[4,5,1-ij]quinoline | 21.2 mg (46%), brown gum |
| NMR | 7.30-7.33 (m, 1H), 7.15-7.24 (m, 2H), 7.05-7.08 (m, 2H), 6.80-6.83 (m, 1H), 4.31-4.31 | | |
| | (m, 2H), 3.85 (s, 1H), 2.82-3.02 (m, 4H), 2.55-2.62 (m, 1H), 2.24-2.31 (m, 3H), 1.65-1.95 | | |
| | (m, 5H). | | |
| 26.93 | 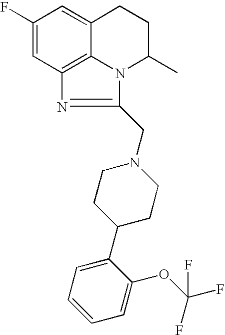 | 8-fluoro-4-methyl-2-{[4- (2- trifluoromethoxyphenyl)- piperidin-1-yl]methyl}-5,6- dihydro-4H- imidazo[4,5,1-ij]quinoline | 50.3 mg (75%), brown gum |
| NMR | 7.22-7.35 (m, 5H), 6.81-6.84 (m, 1H), 4.94-4.97 (m, 1H), 3.85 (s, 1H), 2.80-3.15 (m, | | |
| | 5H), 2.10-2.41 (m, 4H), 1.70-1.85 (m, 4H), 1.50 (d, 3H) | | |
| 26.94 | 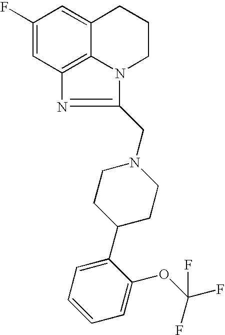 | 8-fluoro-2-{[4-(2- trifluoromethoxy phenyl) piperidin-1-yl]methyl}- 5,6-dihydro-4H- imidazo[4,5,1-ij]quinoline | 55 mg (85%), brown gum |
| NMR | 7.20-7.34 (m, 4H), 6.77-6.81 (m, 1H), 4.26-4.34 (m, 2H), 3.85 (s, 1H), 2.92-3.02 (m, | | |
| | 4H), 2.06-2.31 (m, 3H), 1.65-1.95 (m, 5H). | | |
| 26.95 | 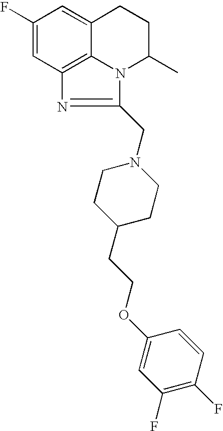 | 8-fluoro-4-methyl-2-{4- [2-(3,4-difluorophenoxy) ethyl] piperidin-1- ylmethyl}-5,6-dihydro- 4H-imidazo[4,5,1- ij]quinoline | 69 mg (97%), brown oil |
| NMR | 7.19-7.23 (dxd, 1H), 6.99-7.06 (m, 1H), 6.70-6.82 (m, 1H), 6.62-6.78 (m, 1H), 6.50-6.58 | | |
| | (m, 1H), 4.91-4.93 (m, 1H), 3.93 (t, 2H), 3.78 (s, 2H), 2.96 (t, 2H), 2.85-3.03 (m, | | |
| | 4H), 2.10-2.16 (m, 4H), 1.68-1.74 (m, 4H), 1.48-1.62 (m, 1H), 1.45 (d, 3H), 1.24-1.32 | | |
| | (m, 2H). | | |
| 26.96 | 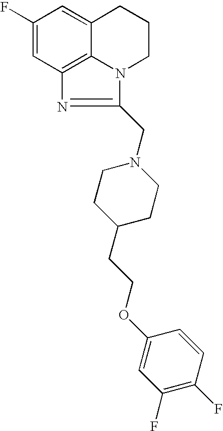 | 8-fluoro-2-{4-[2-(3,4- difluorophenoxy) ethyl]piperidin-1-ylmethyl}- 5,6-dihydro-4H- imidazo[4,5,1-ij]quinoline | 66 mg (96%), brown oil |
| NMR | 7.19-7.23 (dxd, 1H), 7.00-7.07 (m, 1H), 6.77-6.80 (m, 1H), 6.62-6.78 (m, 1H), 6.50-6.58 | | |
| | (m, 1H), 4.26-4.30 (m, 2H), 3.93 (t, 2H), 3.80 (s, 2H), 2.96 (t, 2H), 2.86-2.90 (m, | | |
| | 2H), 2.11-2.26 (m, 4H), 1.42-1.74 (m, 5H), 1.25-1.32 (m, 2H). | | |
| 26.97 | 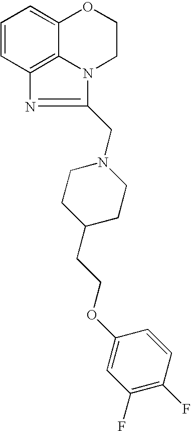 | 2-{4-[2-(3,4- difluorophenoxy) ethyl]piperidin-1-ylmethyl}- 4,5-dihydroimidazo[1,5,4- de][1,4]benzoxazine | 60 mg (91%), yellow solid |
| NMR | 7.30-7.33 (m, 1H), 7.03-7.14 (m, 2H), 6.70-6.75 (m, 2H), 6.50-6.60 (m, 1H), 4.48 (s, | | |
| | 4H), 3.94 (t, 2H), 3.84 (s, 2H), 2.85-2.89 (m, 2H), 2.11-2.19 (m, 2H), 1.68-1.76 (m, | | |
| | 4H), 1.38-1.60 (m, 1H), 1.25-1.31 (2H) | | |
| 26.98 | 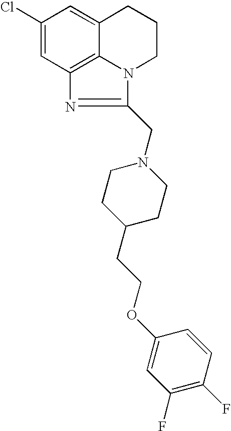 | 8-chloro-2-{4-[2-(3,4- difluorophenoxy) ethyl]piperidin-1-ylmethyl}- 5,6-dihydro-4H- imidazo[4,5,1-ij]quinoline | 71 mg (99%), brown oil |
| NMR | 7.50-7.51 (m, 1H), 6.98-7.09 (m, 2H), 6.65-6.73 (m, 1H), 6.54-6.58 (m, 1H), 4.25-4.29 | | |
| | (m, 2H), 3.92 (t, 2H), 3.78 (s, 2H), 2.94 (t, 2H), 2.83-2.87 (m, 2H), 2.09-2.24 (m, 4H), | | |
| | 1.42-1.74 (m, 5H), 1.25-1.30 (m, 2H). | | |
| 26.99 | 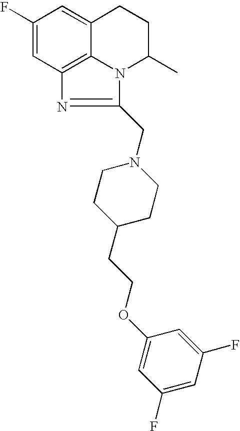 | 8-fluoro-4-methyl-2-{4- [2-(3,5-difluorophenoxy) ethyl] piperidin-1- ylmethyl}-5,6-dihydro- 4H-imidazo[4,5,1- ij]quinoline | 66 mg (96%), brown oil |
| NMR | 7.21-7.28 (dxd, 1H), 6.79-6.83 (dxd, 1H), 6.37-6.43 (m, 3H), 4.92-4.95 (m, 1H), 3.96 | | |
| | (t, 2H), 3.79 (s, 2H), 2.96 (t, 2H), 2.86-3.04 (m, 4H), 2.13-2.17 (m, 4H), 1.69-1.75 (m, | | |
| | 4H), 1.48-1.62 (m, 1H), 1.46 (d, 3H), 1.25-1.33 (m, 2H). | | |
| 26.100 | 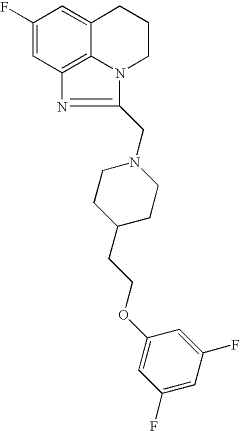 | 8-fluoro-2-{4-[2-(3,5- difluorophenoxy) ethyl]piperidin-1-ylmethyl}- 5,6-dihydro-4H- imidazo[4,5,1-ij]quinoline | 61 mg (92%), brown oil |
| NMR | 7.19-7.23 (dxd, 1H), 6.78-6.82 (m, 1H), 6.38-6.44 (m, 3H), 4.29 (t, 2H), 3.96 (t, 2H), | | |
| | 3.80 (s, 2H), 2.98 (t, 2H), 2.87-2.90 (m, 2H), 2.11-2.26 (m, 4H), 1.42-1.76 (m, 5H), | | |
| | 1.26-1.32 (m, 2H). | | |
| 26.101 | 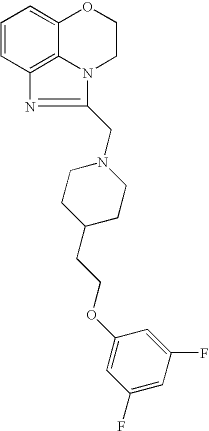 | 2-{4-[2-(3,5- difluorophenoxy) ethyl]piperidin-1-ylmethyl}- 4,5-dihydroimidazo[1,5,4- de][1,4]benzoxazine | 53 mg (83%), yellow solid |
| NMR | 7.30-7.33 (m, 1H), 7.09-7.14 (m, 1H), 6.72-6.75 (m, 1H), 6.37-6.43 (m, 3H), 4.48 (s, | | |
| | 4H), 3.96 (t, 2H), 3.84 (s, 2H), 2.85-2.89 (m, 2H), 2.10-2.18 (m, 2H), 1.69-1.76 (m, | | |
| | 4H), 1.38-1.60 (m, 1H), 1.25-1.30 (2H) | | |
| 26.102 | 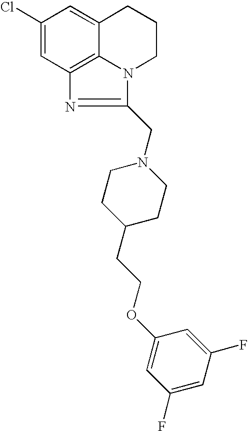 | 8-chloro-2-{4-[2-(3,5- difluorophenoxy) ethyl]piperidin-1-ylmethyl}- 5,6-dihydro-4H- imidazo[4,5,1-ij]quinoline | 70 mg (99%), brown oil |
| NMR | 7.508-7.513 (m, 1H), 6.99 (m, 1H), 6.37-6.43 (m, 3H), 4.28 (t, 2H), 3.95 (t, 2H), 3.78 | | |
| | (s, 2H), 2.95 (t, 2H), 2.84-2.88 (m, 2H), 2.09-2.25 (m, 4H), 1.42-1.75 (m, 5H), 1.26-1.34 | | |
| | (m, 2H). | | |
| 26.103 | 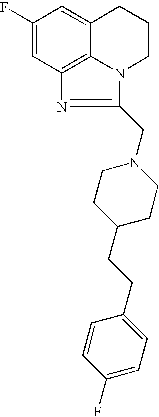 | 8-fluoro-2-{4-[2-(4- fluorophenyl) ethyl]piperidin-1- ylmethyl}-5,6-dihydro- 4H-imidazo[4,5,1- ij]quinoline | 48 mg (74%), yellow solid |
| NMR | 7.21 (dxd, 1H), 7.09-7.14 (m, 2H), 6.93-6.99 (m, 2H), 6.78 (dxd, 1H), 4.27-4.30 (m, | | |
| | 2H), 3.78 (s, 2H), 2.96 (t, 2H), 2.84-2.87 (m, 2H), 2.56-2.62 (m, 2H), 2.21-2.25 (m, | | |
| | 2H), 2.06-2.14 (m, 2H), 1.70-1.73 (m, 2H), 1.51-1.55 (m, 2H), 1.21-1.28 (m, 3H) | | |
| 26.104 | 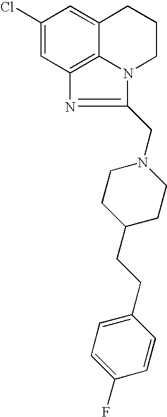 | 8-chloro-2-{4-[2-(4- fluorophenyl) ethyl]piperidin-1- ylmethyl}-5,6-dihydro- 4H-imidazo[4,5,1- ij]quinoline | 60.2 mg (90%), yellow solid |
| NMR | 7.52 (d, 1H), 7.09-7.14 (m, 2H), 6.93-6.99 (m, 3H), 4.26-4.30 (m, 2H), 3.78 (s, 2H), | | |
| | 2.95 (t, 2H), 2.83-2.86 (m, 2H). 2.59 (t, 2H), 2.20-2.24 (m, 2H), 2.06-2.10 (m, 2H), | | |
| | 1.70-1.73 (m, 2H), 1.49-1.55 (m, 2H), 1.18-1.28 (m, 3H) | | |
| 26.105 | 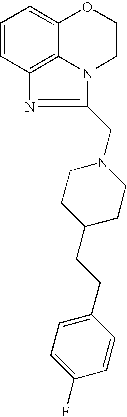 | 2-{4-[2-(4-fluorophenyl) ethyl] piperidin-1- ylmethyl}-4,5- dihydroimidazo[1,5,4- de][1,4]benzoxazine | 48 mg (76%), yellow solid |
| NMR | 7.31-7.33 (m, 1H), 7.09-7.19 (m, 3H), 6.94-6.99 (m, 2H), 6.73-6.75 (d, 1H), 4.48 (s, | | |
| | 4H), 3.83 (s, 2H), 2.83-2.87 (m, 2H), 2.57-2.62 (m, 3H), 2.08-2.11 (m, 2H), 1.72-1.75 | | |
| | (m, 2H), 1.52-1.55 (m, 2H), 1.24-1.29 (m, 2H) | | |
| 26.106 | 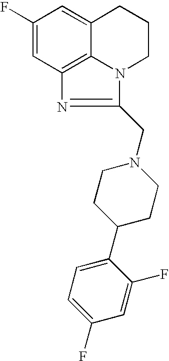 | 8-fluoro-2-{[4-(2,4- difluorophenyl)piperidin- 1-yl]methyl}-5,6-dihydro- 4H-imidazo[4,5,1- ij]quinoline | 33 mg, (64%), yellow solid |
| NMR | 7.16-7.24 (m, 2H), 6.96-6.73-6.82 (m, 3H), 4.30-4.33 (m, 2H), 3.85 (s, 2H), 2.96-3.02 | | |
| | (m, 4H), 2.79-2.96 (m, 1H), 2.22-2.34 (m, 4H), 1.68-1.83 (m, 4H) | | |
| 26.107 | 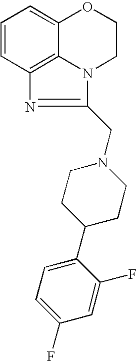 | 2-[(4-2,4-difluorophenyl piperidin-1-yl)methyl]- 4,5-dihydroimidazo[1,5,4- de][1,4]benzoxazine | 39 mg (79%), yellow solid |
| NMR | 7.32-7.34 (m, 1H), 7.10-7.19 (m, 2H), 6.74-6.82 (m, 3H), 4.51 (s, 4H), 3.90 (s, 2H), | | |
| | 2.97-3.02 (m, 2H), 2.80-2.98 (m, 1H), 2.27-2.31 (m, 2H), 1.73-1.82 | | |
| | (m, 4H) | | |
| 26.108 | 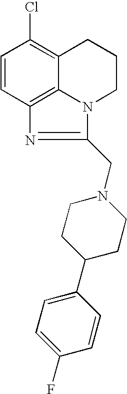 | 7-Chloro-2-[4-(4-fluoro- phenyl)-piperidin- 1-ylmethyl]-5,6- dihydro-4H- imidazo[4,5,1-ij]quinoline | 47.5 mg, (100%), off-white solid |
| NMR | 7.47 (d, 1H), 7.17 (m, 3H), 6.98 (t, 2H), 4.3 (t, 2H), 3.85 (s, 2H), 2.98 (m, 4H), 2.28 | | |
| | (m, 1H), 2.2 (m, 4H), 1.73 (m, 4H) | | |
| 26.109 | 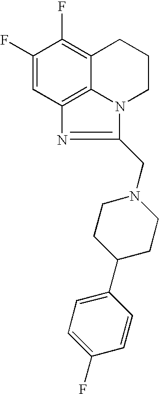 | 7,8-Difluoro-2-[4-(4- fluoro-phenyl)- piperidin-1-ylmethyl]- 5,6-dihydro- 4H-imidazo[4,5,1- ij]quinoline | 11.4 mg, (74%), red gum |
| NMR | 7.35 (m, 1H), 7.17 (m, 2H), 6.97 (t, 2H), 4.31 (t, 2H), 3.83 (s, 2H), 3.05 (m, 4H), 2.52 | | |
| | (m, 1H), 2.26 (m, 4H), 1.72 (m, 4H) | | |
| 26.110 | 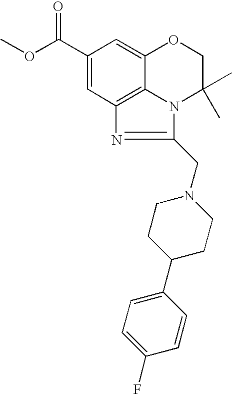 | 2-[4-(4-Fluoro-phenyl)- piperidin-1-ylmethyl]-3,3- dimethyl-3,4-dihydro-5- oxa-1,2a-diaza- acenaphthylene-7- carboxylic acid methyl ester | 27.2 mg, (54%), yellow gum |
| NMR | 8.09 (s, 1H), 7.49 (s, 1H), 7.18 (dd, 2H), 6.99 (t, 2H), 4.12 (s, 2H), 3.95 (s, 3H), 3.87 | | |
| | (s, 2H), 2.95 (br, 2H), 2.58 (m, 1H), 2.3 (br, 2H), 1.85 (br, 2H), 1.74 (m, 8H) | | |
| 26.111 | 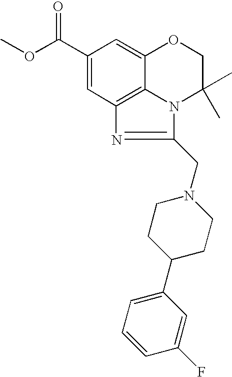 | 2-[4-(3-Fluoro-phenyl)- piperidin-1-ylmethyl]-3,3- dimethyl-3,4-dihydro-5- oxa-1,2a-diaza- acenaphthylene-7- carboxylic acid methyl ester | 38.5 mg, (76%), white gum |
| NMR | 8.09 (s, 1H), 7.49 (s, 1H), 7.27 (dd, 1H), 6.94 (dd, 1H), 6.89 (m, 2H), 1.41 (s, 2H), | | |
| | 3.95 (s, 3H), 3.87 (s, 2H), 2.95 (br, 2H), 2.59 (m, 1H), 2.34 (br, 2H), 1.86 (br, 2H), | | |
| | 1.74 (m, 8H) | | |
| 26.112 | 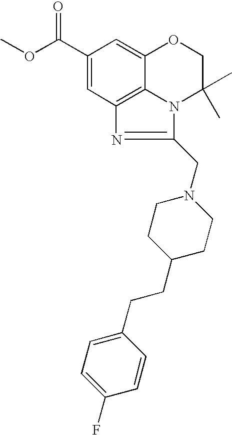 | 2-{4-[2-(4-Fluoro- phenyl)-ethyl]-piperidin- 1-ylmethyl}-3,3- dimethyl-3,4-dihydro-5- oxa-1,2a-diaza- acenaphthylene-7- carboxylic acid methyl ester | 50.2 mg, (94%), white gum |
| NMR | 8.08 (s, 1H), 7.48 (s, 1H), 7.1 (dd, 2H), 6.96 (t, 2H), 4.09 (s, 2H), 3.95 (s, 3H), 3.8 (br, | | |
| | 2H), 2.8 (t, 2H), 2.59 (br, 2H), 2.09 (m, 2H), 1.7 (m, 8H), 1.54 (m, 2H), 1.29 (m, 3H) | | |
| 26.113 | 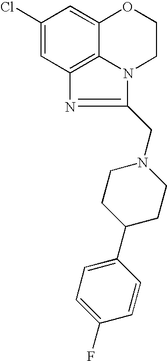 | 7-Chloro-2-[4-(4-fluoro- phenyl)-piperidin- 1-ylmethyl]-3,4- dihydro-5-oxa-1,2a-diaza- acenaphthylene | 54.8 mg, (86%), yellow solid |
| NMR | 7.31 (s, 1H), 7.19 (dd, 2H), 6.98 (t, 2H), 6.76 (s, 1H), 4.49 (s, 4H), 3.87 (s, 2H), 2.96 | | |
| | (br, 2H), 2.56 (m, 1H), 2.26 (td, 2H), 1.82 (br, 2H), 1.7 (td, 2H) | | |
| 26.114 | 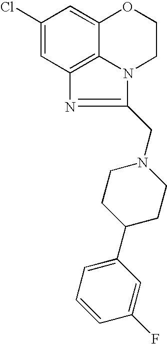 | 7-Chloro-2-[4-(3-fluoro- phenyl)-piperidin- 1-ylmethyl]-3,4- dihydro-5-oxa-1,2a-diaza- acenaphthylene | 55.9 mg, (88%), yellow solid |
| NMR | 7.32 (s, 1H), 7.25 (m, 1H), 6.98 (d, 1H), 6.94 (m, 2H), 6.77 (d, 1H), 4.51 (s, 4H), 3.88 | | |
| | (s, 2H), 2.96 (br, 2H), 2.55 (m, 1H), 2.28 (td, 2H), 1.86 (br, 2H), 1.69 (td, 2H) | | |
| 26.115 | 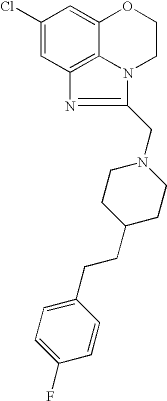 | 7-Chloro-2-{4-[2-(4- fluoro-phenyl)- ethyl]-piperidine-1- ylmethyl}-3,4-dihydro-5- oxa-1,2a-diaza- acenaphthylene | 58.9 mg, (86%), yellow solid |
| NMR | 7.29 (s, 1H), 7.11 (dd, 2H), 6.96 (td, 2H), 6.74 (s, 1H), 4.46 (s, 4H), 3.8 (s, 2H), 2.82 | | |
| | (dd, 2H), 2.58 (td, 2H), 2.1 (m, 2H), 1.74 (br, 2H), 1.52 (m, 2H), 1.25 (m, 3H) | | |
| 26.116 | 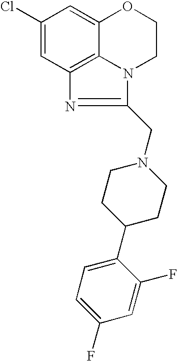 | 7-Chloro-2-[4-(2,4- difluoro-phenyl)- piperidin-1-ylmethyl]-3,4- dihydro-5-oxa-1,2a-diaza- acenaphthylene | 46.9 mg, (70%), yellow solid |
| NMR | 7.31 (s, 1H), 7.18 (m, 1H), 6.79 (m, 3H), 4.49 (s, 4H), 3.88 (s, 2H), 2.96 (br, 2H), 2.84 | | |
| | (m, 1H), 2.3 (td, 2H), 1.75 (m, 4H) | | |
| 26.117 | 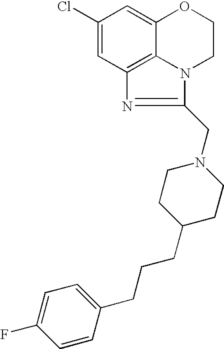 | 7-Chloro-2-{4-[3-(4- fluoro-phenyl)- propyl]-piperidin-1- ylmethyl}-3,4- dihydro-5-oxa-1,2a-diaza- acenaphthylene | 61.6 mg, (87%), yellow solid |
| NMR | 7.29 (s, 1H), 7.12 (dd, 2H), 6.98 (td, 2H), 6.74 (dd, 1H), 4.47 (s, 4H), 3.8 (s, 2H), 2.83 | | |
| | (br, 2H), 2.56 (t, 2H), 2.12 (t, 2H), 1.64 (m, 4H), 1.22 (m, 5H) | | |
| 26.118 | 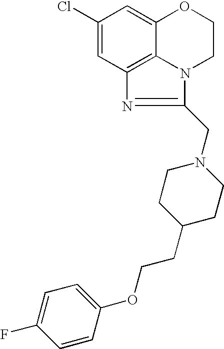 | 7-Chloro-2-{4-[2-(4- fluoro-phenoxy)- ethyl]-piperidin-1- ylmethyl}-3,4- dihydro-5-oxa-1,2a-diaza- acenaphthylene | 64 mg, (90%), yellow solid |

| | |
|---|---|
| NMR | 7.3 (s, 1H), 6.98 (t, 2H), 6.83 (m, 2H), 6.75 (s, 1H), 4.47 (s, 4H), 3.96 (t, 2H), 3.82 (s, |
| | 2H), 2.84 (br, 2H), 2.18 (br, 2H), 1.73 (m, 4H), 1.57 (m, 1H), 1.29 (m, 2H) |

### EXAMPLE 27.1

### 8-Fluoro-2-[4-(4-fluoro-phenyl)-piperidin-1-ylmethyl]-6,6-dimethoxy-5,6-dihydro-4H-imidazo[4,5,1-ij]quinoline

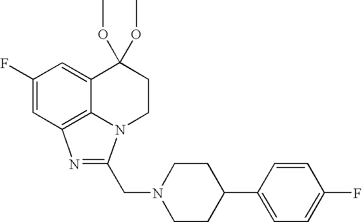

A solution of 2-Chloromethyl-8-fluoro-6,6-dimethoxy-5,6-dihydro-4H-imidazo[4,5,1-ij]quinoline (0.100 g, 0.351 mmol), 4-(4-Fluoro-phenyl)-piperidine (0.091 g, 0.421 mmol), and potassium carbonate (0.145 g, 1.05 mmol) in acetonitrile was stirred overnight at room temperature. The mixture was diluted with ethyl acetate, washed with water and brine, dried over anhydrous sodium sulfate, filtered and concentrated. Purification by flash column chromatography (silica gel, gradient elution with 90% ethyl acetate/hexanes to 3% 2M ammonia/methanol in dichloromethane) afforded a light yellow solid (0.124 g, 83%). ¹H NMR (300 MHz, CDCL₃): 7.35 (dd, 1H), 7.17 (m, 3H), 6.98 (m, 2H), 4.44 (dd, 2H), 3.85 (s, 2H), 3.33 (s, 6H), 3.00 (m, 2H), 2.56 (m, 1H), 2.40 (m, 2H), 2.26 (m, 2H), 1.73 (m, 4H).

### EXAMPLE 28.1

### 8-Fluoro-2-[4-(4-fluoro-phenyl)-piperidin-1-ylmethyl]-4,5,9a,9b-tetrahydroimidazo[4,5,1-ij]quinolin-6-one

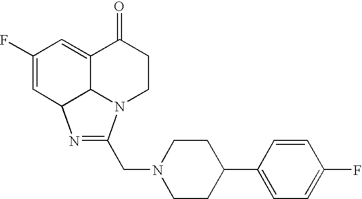

8-Fluoro-2-[4-(4-fluoro-phenyl)-piperidin-1-ylmethyl]-6,6-dimethoxy-5,6-dihydro-4Himidazo[4,5,1-ij]quinoline was dissolved in 4 mL of TFA (20% in dichloromethane) and stirred for 1 h. at room temperature. The solvent was removed in vacuo and the resulting residue was dissolved in ethyl acetate. The organics were washed with saturated sodium bicarbonate, water and brine, dried over anhydrous sodium sulfate, filtered and concentrated. Purification by flash column chromatography (silica gel, gradient elution with 50% ethyl acetate/hexanes to 2% 2M ammonia/methanol in dichloromethane) afforded a white solid (0.078 g, 88%). %). ¹H NMR (300 MHz, CDCL₃): 7.64 (dd, 1H), 7.47 (dd, 1H), 7.18 (m, 2H), 7.00 (m, 2H), 4.73 (t, 2H), 3.91 (s, 2H), 3.14 (t, 2H), 3.00 (m, 2H), 2.56 (m, 1H), 2.32 (m, 2H), 1.87 (m, 2H), 1.75 (m, 2H).

### EXAMPLE 29.1

### 4-Fluoro-1-[4-(4-fluoro-phenyl)-piperidin-1-ylmethyl]-8,9-dihydro-7H-2,7,9a-triaza-benzo[cd]azulen-6-one

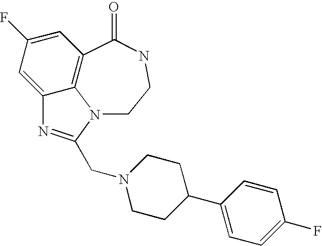

To a flask stirring with methane sulfonic acid (2 mL) in a cold water bath was slowly added 8-Fluoro-2-[4-(4-fluoro-phenyl)-piperidin-1-ylmethyl]4,5,9a,9b-tetrahydroimidazo[4,5,1-ij]quinolin-6-one (0.050 g, 0.131 mmol) and sodium azide (0.011 g, 0.170 mmol). After stirring at room temperature for 3 h., the mixture was cooled by adding ice chips and neutralized with saturated sodium bicarbonate. The aqueous mixture was then extracted with ethyl acetate and the organics were washed with water and brine, dried over anhydrous sodium sulfate, filtered and concentrated. Purification by flash column chromatography (silica gel, elution with 2% 2M ammonia/methanol in dichloromethane) and trituration with dichloromethane afforded a light yellow solid (0.020 g, 38%). ¹H NMR (300 MHz, CDCL₃): 7.87 (dd, 1H), 7.62 (dd, 1H), 7.32 (m, 1H), 7.17 (m, 2H), 6.99 (m, 2H), 5.31 (s, 2H), 4.62 (br, 2H), 3.86 (s, 2H), 3.82 (m, 2H), 2.95 (m, 2H), 2.54 (m, 1H), 2.29 (m, 2H), 1.86 (m, 2H) 1.70 (m, 2H).

### EXAMPLE 30.1

### (i) [4R]— and (ii) [4S]-8-Fluoro-4-methyl-2-{[4(4-trifluoromethyl-phenyl)piperizin-1-yl]methyl}-5,6-dihydro-4H-imidazo[4,5,1-ij]quinoline

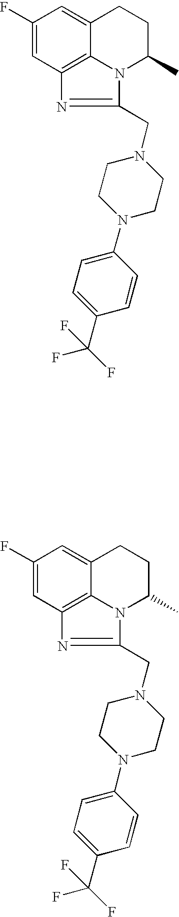

Racemic 8-fluoro-4-methyl-2-{[4(4-trifluoromethyl-phenyl)piperizin-1-yl]methyl}-5,6-dihydro-4H-imidazo[4,5,1-ij]quinoline was separated into its constituent enantiomers using HPLC on a 21.4 mm×250 mm Chiralcel OJ column. Elution was effected with 25% EtOH in petroleum ether at 15 mL/min.

We claim:

1. A compound of formula (I): 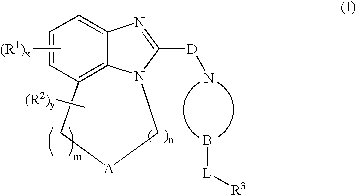
wherein
A is selected from the group consisting of CR⁸R⁹, NR⁵, O, S, SO and SO₂;
B is selected from the group consisting of CH and N;
D is selected from the group consisting of NH, N—C₁₋₆-alkyl, and —CR⁵R⁶)_{z}—, wherein one of the —CR⁵R⁶— groups may be replaced by —C(O)—, NH, or NC₁₋₆-alkyl;
L is selected from the group consisting of a direct bond and —(CR⁵R⁶)_{w}—, wherein when L is —(CR⁵R⁶)_{w}—:
(i) B-L may be unsaturated, or two adjacent carbon atoms may form part of a cyclopropyl ring; or
(ii) one or two CR⁵R⁶ groups may be replaced with O, S, or NR⁵; 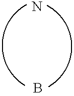
represents a ring selected from the group consisting of azetidine and a 5- to 7-membered ring, which may be unsaturated, wherein the ring may be substituted by one or more R⁴;
R¹, in each instance, is selected from the group consisting of H, F, Cl, Br, I, OH, CN, nitro, C₁₋₆-alkyl, OC₁₋₆-alkyl, C₁₋₆-alkylhalo, OC₁₋₆-alkylhalo, C₂₋₆-alkenyl, OC₂₋₆-alkenyl, C₂₋₆-alkynyl, OC₂₋₆-alkynyl, C₃₋₈-cycloalkyl, C₁₋₆-alkylene-C₃₋₈-cycloalkyl, OC₀₋₆-alkylene-C₃₋₈-cycloalkyl, aryl, heteroaryl, C₁₋₆-alkylenearyl, C₁₋₆-alkyleneheteroaryl, OC₁₋₆-alkylenearyl, OC₁₋₆-alkyleneheteroaryl, C₁₋₆-alkyleneheterocycloalkyl, (CO)R⁵, (CO)OR⁵, C₁₋₆-alkyleneOR⁵, OC₂₋₆-alkyleneOR⁵, C₁₋₆-alkylene(CO)R⁵, OC₁₋₆-alkylene(CO)R⁵, C₁₋₆-alkylenecyano, OC₂₋₆-alkylenecyano, C₀₋₆-alkyleneNR⁶R⁷, OC₂₋₆-alkyleneNR⁶R⁷, C₁₋₆-alkylene(CO)NR⁶R⁷, OC₁₋₆-alkylene(CO)NR⁶R⁷, CO₀₋₆-alkyleneNR⁶(CO)R⁷, OC₂₋₆-alkyleneNR⁶(CO)R⁷, C₀₋₆-alkyleneNR⁶(CO)NR⁶R⁷, C₀₋₆-alkyleneSO₂R⁵, OC₂₋₆-alkyleneSO₂R⁵, C₀₋₆-alkylene(SO₂)NR⁶R⁷, OC₂₋₆-alkylene(SO₂)NR⁶R⁷, C₀₋₆-alkyleneNR⁶(SO₂)R⁷, OC₂₋₆-alkyleneNR⁶(SO₂)R⁷, C₀₋₆-alkyleneNR⁶(SO₂)NR⁶R⁷, OC₂₋₆-alkyleneNR⁶(SO₂)NR⁶R⁷, (CO)NR⁶R⁷and SO₃R⁵, wherein any cyclic group may be further substituted with one or more R² groups;
R² and R⁴, in each instance, are independently selected from the group consisting of H, F, Cl, Br, I, CN, nitro, hydroxy, oxo, C₁₋₆-alkyl, OC₁₋₆-alkyl, C₁₋₆-alkylhalo, OC₁₋₆-alkylhalo, and C₀₋₆-alkyleneNR⁵R⁶;
R³ is a 5- to 12-membered ring system that is optionally substituted by up to three R¹ groups, wherein the ring system may contain one or more heteroatoms independently selected from the group consisting of N, O and S;
R⁵ is selected from the group consisting of H, C₁₋₆-alkyl, aryl, C₃₋₈-cycloalkyl, C₁₋₆-alkylenearyl and C₁₋₆-alkylene-C₃₋₈-cycloalkyl, wherein any cyclic group may be further substituted with one or more independently-selected R² groups;
R⁶ and R⁷ are independently selected from the group consisting of H and C₁₋₆-alkyl;
R⁸ and R⁹ are independently selected from the group consisting of H, —O—(CH₂)₂—O— and —O—(CH₂)₃—O—;
m and n are integers independently selected from the group consisting of 0, 1, 2, 3 and 4, with the proviso that m and n cannot simultaneously be 0;
x and y are integers independently selected from the group consisting of 1, 2, and 3; and
w and z are integers independently selected from the group consisting of 1, 2, 3, 4, 5, and 6;
or a pharmaceutically-acceptable salt, hydrate, solvate, isoform, tautomer, optical isomer, or combination thereof.

2. The compound according to claim 1, wherein m is 0 and n is 2.

3. The compound according to claim 1, wherein A is selected from the group consisting of CH₂ and O.

4. The compound according to claim 1, wherein D is —(CR⁵R⁶)_{z}—.

5. The compound according to claim 4, wherein z is 1.

6. The compound according to claim 4, wherein each of R⁵ and R⁶ is H.

7. The compound according to claim 1, wherein 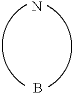
represents piperidine.

8. The compound according to claim 1, wherein R³ is a 5- to 7-membered ring that is optionally substituted by 1-3 R¹groups, wherein the ring may contain one or more heteroatoms independently selected from the group consisting of N, O and S.

9. The compound according to claim 8, wherein R³ is phenyl that is optionally substituted by 1-3 R¹ groups.

10. The compound according to claim 1, wherein
m is 0 and n is 2;
A is CH₂ or O;
R¹ is selected from the group consisting of H, F, Cl, Br, I, nitro, C₁₋₆-alkyl, C₁₋₆-alkylhalo, C₁₋₆-alkylhalo, OC₁₋₆-alkylhalo, aryl, C₁₋₆-alkylenearyl, and OC₁₋₆-alkylenearyl; and
R² is selected from H and C₁₋₆-alkyl.

11. A compound selected from the group consisting of:
8-fluoro-4-methyl-2-[(4-phenylpiperidin-1-yl)methyl]-5,6-dihydro-4H-imidazo[4,5,1-ij]quinoline;
8-fluoro-4-methyl-2-{4-[3-(4-fluorophenyl)propyl]piperidin-1-ylmethyl}-5,6-dihydro-4H-imidazo[4,5,1-ij]quinoline;
8-fluoro-4-methyl-2-{4-[2-(4-fluorophenoxy)ethyl]piperidin-1-ylmethyl}-5,6-dihydro-4H-imidazo[4,5,1-ij]quinoline;
2-[(4-phenylpiperidin-1-yl)methyl]-4,5-dihydroimidazo[1,5,4-de][1,4]benzoxazine;
2-{4-[3-(4-fluorophenyl)propyl]piperidin-1-ylmethyl}-4,5-dihydroimidazo[1,5,4-de][1,4]benzoxazine;
2-{4-[2-(4-fluorophenoxy)ethyl]piperidin-1-ylmethyl}-4,5-dihydroimidazo[1,5,4-de][1,4]benzoxazine;
2-{4-[3-(3-fluoro-5-(trifluoromethyl)-phenyl)propyl]piperidin-1-ylmethyl}-4,5-dihydroimidazo[1,5,4-de][1,4]benzoxazine;
2-[(4-phenylpiperidin-1-yl)methyl]-5,6-dihydro-4H-imidazo[4,5,1-ij]quinoline;
2-{4-[3-(4-fluorophenyl)propyl]piperidin-1-ylmethyl}-5,6-dihydro-4H-imidazo[4,5,1-ij]quinoline;
2-{4-[2-(4-fluorophenoxy)ethyl]piperidin-1-ylmethyl}-5,6-dihydro-4H-imidazo[4,5,1-ij]quinoline;
8-methoxy-2-[(4-phenylpiperidin-1-yl)methyl]-5,6-dihydro-4H-imidazo[4,5,1-ij]quinoline;
8-methyl-2-[(4-phenylpiperidin-1-yl)methyl]-5,6-dihydro-4H-imidazo[4,5,1-ij]quinoline;
8-methyl-2-{4-[3-(4-fluorophenyl)propyl]piperidin-1-ylmethyl}-5,6-dihydro-4H-imidazo[4,5,1-ij]quinoline;
8-methyl-2-{4-[2-(4-fluorophenoxy)ethyl]piperidin-1-ylmethyl}-5,6-dihydro-4H-imidazo[4,5,1-ij]quinoline;
8-methyl-2-{4-[3-(3-fluoro-5(trifluoromethyl)-phenyl)propyl]piperidin-1-ylmethyl}-5,6-dihydro-4H-imidazo[4,5,1-ij]quinoline;
8-methyl-2-[(4-benzylpiperidin-1-yl)methyl]-5,6-dihydro-4H-imidazo[4,5,1-ij]quinoline;
8-methyl-2-{[4-(4-bromophenyl)piperidin-1-yl]methyl}-5,6-dihydro-4H-imidazo[4,5,1-ij]quinoline;
8-methyl-2-{[4-(4-cyanophenyl)piperidin-1-yl]methyl}-5,6-dihydro-4H-imidazo[4,5,1-ij]quinoline;
8-methyl-2-{[4-(4-chlorophenyl)piperidin-1-yl]methyl}-5,6-dihydro-4H-imidazo[4,5,1-ij]quinoline;
8-methyl-2-[(4-phenoxypiperidin-1-yl)methyl]-5,6-dihydro-4H-imidazo[4,5,1-ij]quinoline;
8-methyl-2-{[4-(3-hydroxyphenyl)piperidin-1-yl]methyl}-5,6-dihydro-4H-imidazo[4,5,1-ij]quinoline;
8-fluoro-4-methyl-2-[4-(4-fluoro-phenyl)-piperidin-1-ylmethyl]-4-methyl-5,6-dihydro-4H-imidazo[4,5,1-ij]quinoline;
8-fluoro-4-methyl-2-[4-(4-trifluoromethylphenyl)-piperidin-1-ylmethyl]-5,6-dihydro-4H-imidazo[4,5,1-ij]quinoline;
8-fluoro-4-methyl-2-[4-(4-bromo-phenyl)-piperidin-1-ylmethyl]-5,6-dihydro-4H-imidazo[4,5,1-ij]quinoline;
8-fluoro-4-methyl-2-[4-(4-cyano-phenyl)-piperidin-1-ylmethyl]-5,6-dihydro-4H-imidazo[4,5,1-ij]quinoline;
8-fluoro-4-methyl-2-[4-(4-methylphenyl)-piperidin-1-ylmethyl]-5,6-dihydro-4H-imidazo[4,5,1-ij]quinoline;
8-fluoro-4-methyl-2-[4-(4-methoxyphenyl)-piperidin-1-ylmethyl]-5,6-dihydro-4H-imidazo[4,5,1-ij]quinoline;
8-fluoro-4-methyl-2-[4-(2-methoxyphenyl)-piperidin-1-ylmethyl]-5,6-dihydro-4H-imidazo[4,5,1-ij]quinoline;
8-fluoro-4-methyl-2-[4-(2-methylphenyl)-piperidin-1-ylmethyl]-5,6-dihydro-4H-imidazo[4,5,1-ij]quinoline;
8-fluoro-2-[(4-phenylpiperidin-1-yl)methyl]-5,6-dihydro-4H-imidazo[4,5,1-ij]quinoline;
8-fluoro-2-[(4-benzylpiperidin-1-yl)methyl]-5,6-dihydro-4H-imidazo[4,5,1-ij]quinoline;
8-fluoro-2-[4-(3-phenyl propyl)piperidin-1-ylmethyl]-5,6-dihydro-4H-imidazo[4,5,1-ij]quinoline;
8-fluoro-2-{4-[3-(4-fluorophenyl)propyl]piperidin-1-ylmethyl}-5,6-dihydro-4H-imidazo[4,5,1-ij]quinoline;
8-fluoro-2-{4-[2-(4-fluorophenoxy)ethyl]piperidin-1-ylmethyl}-5,6-dihydro-4H-imidazo[4,5,1-ij]quinoline;
8-fluoro-2-{[4-(4-fluorophenyl)piperidin-1-yl]methyl}-5,6-dihydro-4H-imidazo[4,5,1-ij]quinoline;
8-fluoro-2-{[4-(4-trifluoromethyl-phenyl)piperidin-1-yl]methyl}-5,6-dihydro-4H-imidazo[4,5,1-ij]quinoline;
8-chloro-2-{[4-(4-fluorophenyl)piperidin-1-yl]methyl}-5,6-dihydro-4H-imidazo[4,5,1-ij]quinoline;
8-chloro-2-{[4-(4-trifluoromethyl-phenyl)piperidin-1-yl]methyl}-5,6-dihydro-4H-imidazo[4,5,1-ij]quinoline;
8-chloro-2-{4-[3-(4-fluorophenyl)propyl]piperidin-1-ylmethyl}-5,6-dihydro-4H-imidazo[4,5,1-ij]quinoline;
8-fluoro-4-methyl-2-[4-(2-trifluoromethylphenyl)-piperidin-1-ylmethyl]-5,6-dihydro-4H-imidazo[4,5,1-ij]quinoline;
8-fluoro-4-methyl-2-[4-(3-fluoro-phenyl)-piperidin-1-ylmethyl]-5,6-dihydro-4H-imidazo[4,5,1-ij]quinoline;
8-fluoro-4-methyl-2-[4-(3-trifluoromethylphenyl)-piperidin-1-ylmethyl]-5,6-dihydro-4H-imidazo[4,5,1-ij]quinoline;
8-fluoro-4-methyl-2-{[4-(4-fluorophenyl)-3,6-dihydropyridin-1(2H)-yl]methyl}-5,6-dihydro-4H-imidazo[4,5,1-ij]quinoline;
8-fluoro-2-{[4-(4-fluorophenyl)-3,6-dihydropyridin-1(2H)-yl]methyl}-5,6-dihydro-4H-imidazo[4,5,1-ij]quinoline;
8-chloro-2-{[4-(4-fluorophenyl)-3,6-dihydropyridin-1(2H)-yl]methyl}-5,6-dihydro-4H-imidazo[4,5,1-ij]quinoline;
8-fluoro-4-methyl-2-{[4-(2,5-difluorophenyl)-3,6-dihydropyridin-1(2H)-yl]methyl}-5,6-dihydro-4H-imidazo[4,5,1-ij]quinoline;
8-fluoro-2-{[4-(2,5-difluorophenyl)-3,6-dihydropyridin-1(2H)-yl]methyl}-5,6-dihydro-4H-imidazo[4,5,1-ij]quinoline;
8-chloro-2-{[4-(2,5-difluorophenyl)-3,6-dihydropyridin-1(2H)-yl]methyl}-5,6-dihydro-4H-imidazo[4,5,1-ij]quinoline;
8-fluoro-4-methyl-2-[4-(4-chlorophenyl)-piperidin-1-ylmethyl]-5,6-dihydro-4H-imidazo[4,5,1-ij]quinoline;
8-fluoro-4-methyl-2-[4-(3-chlorophenyl)-piperidin-1-ylmethyl]-5,6-dihydro-4H-imidazo[4,5,1-ij]quinoline;
8-fluoro-4-methyl-2-[4-(3,5-bis(trifluoromethyl)-phenyl)-piperidin-1-ylmethyl]-5,6-dihydro-4H-imidazo[4,5,1-ij]quinoline;
8-fluoro-4-methyl-2-[4-benzylphenyl)-piperidin-1-ylmethyl]-5,6-dihydro-4H-imidazo[4,5,1-ij]quinoline;
8-fluoro-4-methyl-2-[4-(3-methoxyphenyl)-piperidin-1-ylmethyl]-5,6-dihydro-4H-imidazo[4,5,1-ij]quinoline;
8-fluoro-4-methyl-2-[4-(3-methylphenyl)-piperidin-1-ylmethyl]-5,6-dihydro-4H-imidazo[4,5,1-ij]quinoline;
8-fluoro-4-methyl-2-[4-(2-chlorophenoxy)-piperidin-1-ylmethyl]-5,6-dihydro-4H-imidazo[4,5,1-ij]quinoline;
8-fluoro-4-methyl-2-{[4(4-fluorophenyl)piperizin-1-yl]methyl}-5,6-dihydro-4H-imidazo[4,5,1-ij]quinoline;
8-fluoro-4-methyl-2-{[4(4-trifluoromethyl-phenyl)piperizin-1-yl]methyl}-5,6-dihydro-4H-imidazo[4,5,1-ij]quinoline;
8-fluoro-4-methyl-2-{[4(2,4-difluorophenyl)piperizin-1-yl]methyl}-5,6-dihydro-4H-imidazo[4,5,1-ij]quinoline;
8-fluoro-4-methyl-2-[4-(2-fluoro-phenyl)-piperidin-1-ylmethyl]-5,6-dihydro-4H-imidazo[4,5,1-ij]quinoline;
8-fluoro-4-methyl-2-[4-phenoxy-piperidin-1-ylmethyl]-5,6-dihydro-4H-imidazo[4,5,1-ij]quinoline;
8-fluoro-4-methyl-2-[3-phenyl-piperazin-1-ylmethyl]-5,6-dihydro-4H-imidazo[4,5,1-ij]quinoline;
8-fluoro-4-methyl-2-{[4-(2,5-difluorophenyl)piperidin-1-yl]methyl}-5,6-dihydro-4H-imidazo[4,5,1-ij]quinoline;
8-fluoro-4-methyl-2-{[4-(4-bromophenyl)-3,6-dihydropyridin-1(2H)-yl]methyl}-5,6-dihydro-4H-imidazo[4,5,1-ij]quinoline;
8-fluoro-2-{[4-(4-bromophenyl)-3,6-dihydropyridin-1(2H)-yl]methyl}-5,6-dihydro-4H-imidazo[4,5,1-ij]quinoline;
8-fluoro-4-methyl-2-{[4-(4-trifluoromethoxyphenyl)-3,6-dihydropyridin-1(2H)-yl]methyl}-5,6-dihydro-4H-imidazo[4,5,1-ij]quinoline;
8-fluoro-2-{[4-(4-trifluoromethoxyphenyl)-3,6-dihydropyridin-1(2H)-yl]methyl}-5,6-dihydro-4H-imidazo[4,5,1-ij]quinoline;
2-[(4-(4-fluorophenyl)piperidin-1-yl)methyl]-5,6-dihydro-4H-imidazo[4,5,1-ij]quinoline;
2-[(4-(4-trifluoromethylphenyl)piperidin-1-yl)methyl]-5,6-dihydro-4H-imidazo[4,5,1-ij]quinoline;
2-[(4-(4-fluorophenylpiperidin-1-yl)methyl]-4,5-dihydroimidazo[1,5,4-de][1,4]benzoxazine;
2-[(4-(4-trifluoromethylphenylpiperidin-1-yl)methyl]-4,5-dihydroimidazo[1,5,4-de][1,4]benzoxazine;
[4R]-8-fluoro-4-methyl-2-[(4-phenylpiperidin-1-yl)methyl]-5,6-dihydro-4H-imidazo[4,5,1-ij]quinoline;
[4S]-8-fluoro-4-methyl-2-[(4-phenylpiperidin-1-yl)methyl]-5,6-dihydro-4H-imidazo[4,5,1-ij]quinoline;
8-fluoro-4-methyl-2-{[4-(4-trifluoromethoxyphenyl)piperidin-1-yl]methyl}-5,6-dihydro-4H-imidazo[4,5,1-ij]quinoline;
8-fluoro-2-{[4-(4-trifluoromethoxyphenyl)piperidin-1-yl]methyl}-5,6-dihydro-4H-imidazo[4,5,1-ij]quinoline;
8-chloro-2-[(4-(2-trifluoromethylphenyl)piperidin-1-yl)methyl]-5,6-dihydro-4H-imidazo[4,5,1-ij]quinoline;
8-chloro-2-[(4-(3-trifluoromethylphenyl)piperidin-1-yl)methyl]-5,6-dihydro-4H-imidazo[4,5,1-ij]quinoline;
8-chloro-2-[(4-(4-chlorophenyl)piperidin-1-yl)methyl]-5,6-dihydro-4H-imidazo[4,5,1-ij]quinoline;
8-chloro-2-[(4-(3-chlorophenyl)piperidin-1-yl)methyl]-5,6-dihydro-4H-imidazo[4,5,1-ij]quinoline;
8-chloro-2-[(4-(2-fluorophenyl)piperidin-1-yl)methyl]-5,6-dihydro-4H-imidazo[4,5,1-ij]quinoline;
8-chloro-2-[(4-(3-fluorophenyl)piperidin-1-yl)methyl]-5,6-dihydro-4H-imidazo[4,5,1-ij]quinoline;
8-chloro-2-[(4-(2-methylphenyl)piperidin-1-yl)methyl]-5,6-dihydro-4H-imidazo[4,5,1-ij]quinoline;
8-chloro-2-[(4-(3-methylphenyl)piperidin-1-yl)methyl]-5,6-dihydro-4H-imidazo[4,5,1-ij]quinoline;
8-chloro-2-[(4-(4-methylphenyl)piperidin-1-yl)methyl]-5,6-dihydro-4H-imidazo[4,5,1-ij]quinoline;
8-chloro-2-[(4-(3-methoxyphenyl)piperidin-1-yl)methyl]-5,6-dihydro-4H-imidazo[4,5,1-ij]quinoline;
8-chloro-2-[(4-(4-methoxyphenyl)piperidin-1-yl)methyl]-5,6-dihydro-4H-imidazo[4,5,1-ij]quinoline;
8-fluoro-2-{[4-(3-fluorophenyl)piperidin-1-yl]methyl}-5,6-dihydro-4H-imidazo[4,5,1-ij]quinoline;
8-chloro-2-[(4-(2-methoxyphenyl)piperidin-1-yl)methyl]-5,6-dihydro-4H-imidazo[4,5,1-ij]quinoline;
8-fluoro-4-methyl-2-[4-(2,4-difluoro-phenyl)-piperidin-1-ylmethyl]-5,6-dihydro-4H-imidazo[4,5,1-ij]quinoline;
8-fluoro-4-methyl-2-[4-(4-fluoro-3-trifluoromethyl-phenyl)-piperidin-1-ylmethyl]-5,6-dihydro-4H-imidazo[4,5,1-ij]quinoline;
8-fluoro-2-[4-(4-fluoro-3-trifluoromethyl-phenyl)-piperidin-1-ylmethyl]-5,6-dihydro-4H-imidazo[4,5,1-ij]quinoline;
8-fluoro-4-methyl-2-{[4-(3-trifluoromethoxyphenyl)piperidin-1-yl]methyl}-5,6-dihydro-4H-imidazo[4,5,1-ij]quinoline;
8-fluoro-2-{[4-(3-trifluoromethoxyphenyl)piperidin-1-yl]methyl}-5,6-dihydro-4H-imidazo[4,5,1-ij]quinoline;
8-fluoro-4-methyl-2-{[4-(2-trifluoromethoxyphenyl)piperidin-1-yl]methyl}-5,6-dihydro-4H-imidazo[4,5,1-ij]quinoline;
8-fluoro-2-{[4-(2-trifluoromethoxy phenyl)piperidin-1-yl]methyl}-5,6-dihydro-4H-imidazo[4,5,1-ij]quinoline;
8-fluoro-4-methyl-2-{4-[2-(3,4-difluorophenoxy)ethyl]piperidin-1-ylmethyl}-5,6-dihydro-4H-imidazo[4,5,1-ij]quinoline;
8-fluoro-2-{4-[2-(3,4-difluorophenoxy)ethyl]piperidin-1-ylmethyl}-5,6-dihydro-4H-imidazo[4,5,1-ij]quinoline;
2-{4-[2-3,4-difluorophenoxy)ethyl]piperidin-1-ylmethyl}-4,5-dihydroimidazo[1,5,4-de][1,4]benzoxazine;
8-chloro-2-{4-[2-3,4-difluorophenoxy)ethyl]piperidin-1-ylmethyl}-5,6-dihydro-4H-imidazo[4,5,1-ij]quinoline;
8-fluoro-4-methyl-2-{4-[2-(3,5-difluorophenoxy)ethyl]piperidin-1-ylmethyl}-5,6-dihydro-4H-imidazo[4,5,1-ij]quinoline;
8-fluoro-2-{4-[2-(3,5-difluorophenoxy)ethyl]piperidin-1-ylmethyl}-5,6-dihydro-4H-imidazo[4,5,1-ij]quinoline;
2-{4-[2-(3,5-difluorophenoxy)ethyl]piperidin-1-ylmethyl}-4,5-dihydroimidazo[1,5,4-de][1,4]benzoxazine;
8-chloro-2-{4-[2-(3,5-difluorophenoxy)ethyl]piperidin-1-ylmethyl}-5,6-dihydro-4H-imidazo[4,5,1-ij]quinoline;
8-fluoro-2-{4-[2-(4-fluorophenyl)ethyl]piperidin-1-ylmethyl}-5,6-dihydro-4H-imidazo[4,5,1-ij]quinoline;
8-chloro-2-{4-[2-(4-fluorophenyl)ethyl]piperidin-1-ylmethyl}-5,6-dihydro-4H-imidazo[4,5,1-ij]quinoline;
2-{4-[2-(4-fluorophenyl)ethyl]piperidin-1-ylmethyl}-4,5-dihydroimidazo[1,5,4-de][1,4]benzoxazine;
8-fluoro-2-{[4-(2,4-difluorophenyl)piperidin-1-yl]methyl}-5,6-dihydro-4H-imidazo[4,5,1-ij]quinoline;
2-[(4-2,4-difluorophenyl piperidin-1-yl)methyl]-4,5-dihydroimidazo[1,5,4-de][1,4]benzoxazine;
7-chloro-2-[4-(4-fluoro-phenyl)-piperidin-1-ylmethyl]-5,6-dihydro-4H-imidazo[4,5,1-ij]quinoline;
7,8-difluoro-2-[4-(4-fluoro-phenyl)-piperidin-1-ylmethyl]-5,6-dihydro-4H-imidazo[4,5,1-ij]quinoline;
2-[4-(4-fluoro-phenyl)-piperidin-1-ylmethyl]-3,3-dimethyl-3,4-dihydro-5-oxa-1,2a-diaza-acenaphthylene-7-carboxylic acid methyl ester;
2-[4-(3-fluoro-phenyl)-piperidin-1-ylmethyl]-3,3-dimethyl-3,4-dihydro-5-oxa-1,2a-diaza-acenaphthylene-7-carboxylic acid methyl ester;
2-{4-[2-(4-fluoro-phenyl)-ethyl]-piperidin-1-ylmethyl}-3,3-dimethyl-3,4-dihydro-5-oxa-1,2a-diaza-acenaphthylene-7-carboxylic acid methyl ester;
7-chloro-2-[4-(4-fluoro-phenyl)-piperidin-1-ylmethyl]-3,4-dihydro-5-oxa-1,2a-diaza-acenaphthylene;
7-chloro-2-[4-(3-fluoro-phenyl)-piperidin-1-ylmethyl]-3,4-dihydro-5-oxa-1,2a-diaza-acenaphthylene;
7-chloro-2-{4-[2-(4-fluoro-phenyl)-ethyl]-piperidin-1-ylmethyl}-3,4-dihydro-5-oxa-1,2a-diaza-acenaphthylene;
7-chloro-2-[4-(2,4-difluoro-phenyl)-piperidin-1-ylmethyl]-3,4-dihydro-5-oxa-1,2a-diaza-acenaphthylene;
7-chloro-2-{4-[3-(4-fluoro-phenyl)-propyl]-piperidin-1-ylmethyl}-3,4-dihydro-5-oxa-1,2a-diaza-acenaphthylene;
7-chloro-2-{4-[2-(4-fluoro-phenoxy)-ethyl]-piperidin-1-ylmethyl}-3,4-dihydro-5-oxa-1,2a-diaza-acenaphthylene;
8-fluoro-2-[4-(4-fluoro-phenyl)-piperidin-1-ylmethyl]-6,6-dimethoxy-5,6-dihydro-4H-imidazo[4,5,1-ij]quinoline;
8-fluoro-2-[4-(4-fluoro-phenyl)-piperidin-1-ylmethyl]-4,5,9a,9b-tetrahydroimidazo[4,5,1-ij]quinolin-6-one;
4-fluoro-1-[4-(4-fluoro-phenyl)-piperidin-1-ylmethyl]-8,9-dihydro-7H-2,7,9a-triaza-benzo[cd]azulen-6-one;
[4R]-8-fluoro-4-methyl-2-{[4(4-trifluoromethyl-phenyl)piperizin-1-yl]methyl}-5,6-dihydro-4H-imidazo[4,5,1-ij]quinoline; and
[4S]-8-fluoro-4-methyl-2-{[4(4-trifluoromethyl-phenyl)piperizin-1-yl]methyl}-5,6-dihydro-4H-imidazo[4,5,1-ij]quinoline.

12. A pharmaceutical composition comprising a compound according to claim 1 and a pharmaceutically acceptable carrier or excipient.

13. A method for the treatment or prevention of neurological and psychiatric disorders associated with glutamate dysfunction in an animal in need of such treatment, comprising the step of administering to said animal a therapeutically effective amount of a compound according to claim 1.

14. A method for the treatment or prevention of neurological and psychiatric disorders associated with glutamate dysfunction in an animal in need of such treatment, comprising the step of administering to said animal a therapeutically effective amount of a compound of formula I in the form of a pharmaceutical composition according to claim 12.

15. The method according to claim 13 or 14, wherein the neurological and psychiatric disorders are selected from the group consisting of cerebral deficit subsequent to cardiac bypass surgery and grafting, stroke, cerebral ischemia, spinal cord trauma, head trauma, perinatal hypoxia, cardiac arrest, hypoglycemic neuronal damage, dementia, AIDS-induced dementia, Alzheimer's disease, Huntington's Chorea, amyotrophic lateral sclerosis, ocular damage, retinopathy, cognitive disorders, idiopathic and drug-induced Parkinson's disease, muscular spasms and disorders associated with muscular spasticity including tremors, epilepsy, convulsions, migraine, urinary incontinence, substance tolerance, substance withdrawal, psychosis, schizophrenia, anxiety, mood disorders, circadian rhythm disorders, trigeminal neuralgia, hearing loss, tinnitus, macular degeneration of the eye, emesis, brain edema, pain, tardive dyskinesia, sleep disorders, attention deficit/hyperactivity disorder, and conduct disorder.

16. The pharmaceutical composition according to claim 12, for use in the prevention and/or treatment of mGluR2 receptor-mediated disorders.

17. A compound according to claim 1 for use in therapy.

18. The compound according to claim 17, for use in the prevention and/or treatment of mGluR2 receptor-mediated disorders.

19. The use of a compound according to claim 1 in the manufacture of a medicament for the use in the prevention and/or treatment of mGluR2 receptor-mediated disorders.

20. A process for the preparation of a compound of formula I according to claim 1, said process comprising the step of reacting a precursor compound according to formula (i): 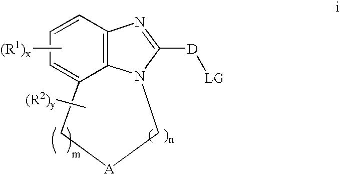
with a precursor compound according to formula (ii): 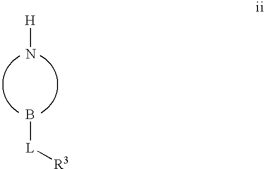
to give the compound according to formula I: 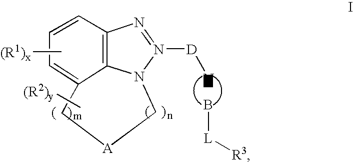
wherein R¹, R², R³, A, D, L, m, n, x, and y are as defined in claim 1, and wherein LG is a leaving group.
